SEQUENCE LISTING (57 sequences)

SEQ ID NO: 1 - DNA, 57 bp, Artificial Sequence
  Synthetic construct
atgggatggtcatgtatcatcctttttctagtagcaactgcaaccggtgtacattcc

SEQ ID NO: 2 - DNA, 321 bp, Artificial Sequence
  Synthetic construct
gacatcaagatgacccagtctccatcttccatgtatgcatctctaggagagagagtcactatcacttgcaaggcgagtccggacattaatagctatttaagctggttccagcagaaaccagggaaatctcctaagaccctgatctatcgtgcaaacagattggttgatggggtcccatcaaggttcagtggcggtggatctgggcaagattattctctcaccatcaacagcctggagtatgaagatatgggaatttattattgtctacagtatgatgaatttccgtacacgttcggaggggggaccaagctggaaatgaaa

SEQ ID NO: 3 - DNA, 321 bp, Artificial Sequence
  Synthetic construct
gacatccagatgacccagtctccatcctccctgtctgcatctgtaggagacagagtcaccatcacttgcaaggcgagtccggacattaatagctatttaagctggtttcagcagaggccaggccaatctccaaggcgcctaatttatcgtgcaaacagattggttgatggggtcccagacaggttcagtggcagtgggtcaggcactgatttcacactgaaaatcagcagggtggaggctgaggatgttggagtttattactgtctacagtatgatgaatttccgtacacgttcggccaagggaccaaggtggaaatcaaa

SEQ ID NO: 4 - DNA, 321 bp, Artificial Sequence
  Synthetic construct
gatattgtgatgacccagactccactctccctgcccgtcacccctggagagccggcctccatctcctgcagggcaagtaagagcattagcaaatatttagcctggtaccagcagaaacctggccaggctcccaggctcctcatctattctggatccactttgcaatctgggatcccacctcgattcagtggcagcgggtatggaacagattttaccctcacaattaataacatagaatctgaggatgctgcatattacttctgtcaacagcatgatgaatccccgtacacgttcggcgaggggaccaaggtggaaatcaaa

SEQ ID NO: 5 - DNA, 54 bp, Artificial Sequence
  Synthetic construct
ggctccacctctggatccggcaagcccggatctggcgagggatccaccaagggc

SEQ ID NO: 6 - DNA, 54 bp, Artificial Sequence; identical to SEQ ID NO: 5
  Synthetic construct

SEQ ID NO: 7 - DNA, 84 bp, Artificial Sequence
  Synthetic construct
ggtggtggtggtagcggctccacctctggatccggcaagcccggatctggcgagggatccaccaagggcggaggaggaggaagt

SEQ ID NO: 8 - DNA, 357 bp, Artificial Sequence
  Synthetic construct
gaagtgaaactagtggagtctgggggaggcttagtgaagcctggagggtccctgaaactctcctgtgcagcctctggattcactttcagtagctatgccatgtcttgggttcgccagattccagagaagaggctggagtgggtcgcatccattagtcgtggtggtaccacctactatccagacagtgtgaagggccgattcaccatctccagagataatgtcaggaacatcctgtacctgcaaatgagcagtctgaggtctgaggacacggccatgtattactgtggaagatatgattacgacgggtactatgcaatggactactggggtcaaggaacctcagtcaccgtctcctca

SEQ ID NO: 9 - DNA, 357 bp, Artificial Sequence
  Synthetic construct
gaagtgcagctggtgcagtctggagcagaggtgaaaaagcccggggagtctctgaggatctcctgtaagggttctggattcactttcagtagctatgccatgtcttggatcaggcagtccccatcgagaggccttgagtggctgggttccattagtcgtggtggtaccacctactatccagacagtgtgaagggcagattcaccatctccagagacaacgccaagaactcactgtatctgcaaatgaacagcctgagagccgaggacacggctgtgtattactgtggaagatatgattacgacgggtactatgcaatggactactggggccagggaacgctggtcaccgtcagctca

SEQ ID NO: 10 - DNA, 348 bp, Artificial Sequence
  Synthetic construct
caggtgcagctgcaggagtcgggcccaggactggtgaagccttcacagaccctgtccctcacctgcactgtctctggttatgcattcactgcctacaacatacactgggtgcgacaggcccctggacaagggcttgagtggatgggttcttttgatccttacgatggtggtagtagttacaaccagaagttcaaggacagactcaccatctccaaggacacctccaaaaaccaggtggtccttacaatgaccaacatggaccctgtggacacagccacgtattactgtgcaagagggtggtactactttgactactggggccacggaaccctggtcaccgtctcctca

SEQ ID NO: 11 - DNA, 42 bp, Artificial Sequence
  Synthetic construct
gtcgacgagtccaaatatggtcccccatgcccaccatgccca

SEQ ID NO: 12 - DNA, 399 bp, Artificial Sequence
  Synthetic construct
ggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtccctaggtaaaatg

SEQ ID NO: 13 - DNA, 654 bp, Artificial Sequence
  Synthetic construct
gcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtccctaggtaaaatg

SEQ ID NO: 14 - DNA, 204 bp, Artificial Sequence
  Synthetic construct
ttttgggtgctggtggtggttggtggagtcctggcttgctatagcttgctagtaacagtggcctttattattttctgggtgaggagtaagaggagcaggggcggacacagtgactacatgaacatgactccccgccgccctgggcccacccgcaagcattaccagccctatgccccaccacgcgacttcgcagcctatcgctcc

SEQ ID NO: 15 - DNA, 126 bp, Artificial Sequence
  Synthetic construct
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 16 - DNA, 339 bp, Artificial Sequence
  Synthetic construct
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 17 - DNA, 7489 bp, Artificial Sequence
  Synthetic construct
gtccgaaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatgcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgatatcctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctctagagccacc

SEQ ID NO: 18 - protein, 19 aa, Artificial Sequence
  Synthetic construct
MGWSCIILFLVATATGVHS

SEQ ID NO: 19 - protein, 107 aa, Artificial Sequence
  Synthetic construct
DIKMTQSPSSMYASLGERVTITCKASPDINSYLSWFQQKPGKSPKTLIYRANRLVDGVPSRFSGGGSGQDYSLTINSLEYEDMGIYYCLQYDEFPYTFGGGTKLEMK

SEQ ID NO: 20 - protein, 107 aa, Artificial Sequence
  Synthetic construct
DIQMTQSPSSLSASVGDRVTITCKASPDINSYLSWFQQRPGQSPRRLIYRANRLVDGVPDRFSGSGSGTDFTLKISRVEAEDVGVYYCLQYDEFPYTFGQGTKVEIK

SEQ ID NO: 21 - protein, 107 aa, Artificial Sequence
  Synthetic construct
DIVMTQTPLSLPVTPGEPASISCRASKSISKYLAWYQQKPGQAPRLLIYSGSTLQSGIPPRFSGSGYGTDFTLTINNIESEDAAYYFCQQHDESPYTFGEGTKVEIK

SEQ ID NO: 22 - protein, 18 aa, Artificial Sequence
  Synthetic construct
GSTSGSGKPGSGEGSTKG

SEQ ID NO: 23 - protein, 18 aa, Artificial Sequence
  Synthetic construct
GSTSGSGKPGSGEGSTKG

SEQ ID NO: 24 - protein, 28 aa, Artificial Sequence
  Synthetic construct
GGGGSGSTSGSGKPGSGEGSTKGGGGGS

SEQ ID NO: 25 - protein, 119 aa, Artificial Sequence
  Synthetic construct
EVKLVESGGGLVKPGGSLKLSCAASGFTESSYAMSWVRQIPEKRLEWVASISRGGTTYYPDSVKGRFTISRDNVRNILYLQMSSLRSEDTAMYYCGRYDYDGYYAMDYWGQGTSVTVSS

SEQ ID NO: 26 - protein, 119 aa, Artificial Sequence
  Synthetic construct
EVQLVQSGAEVKKPGESLRISCKGSGFTFSSYAMSWIRQSPSRGLEWLGSISRGGTTYYPDSVKGRFTISRDNAKNSLYLQMNSLRAEDTAVYYCGRYDYDGYYAMDYWGQGTLVTVSS

SEQ ID NO: 27 - protein, 116 aa, Artificial Sequence
  Synthetic construct
QVQLQESGPGLVKPSQTLSLTCTVSGYAFTAYNIHWVRQAPGQGLEWMGSFDRYDGGSSYNQKFKDRLTISKDTSKNQVVLTMTNMDPVDTATYYCARGWYYFDYWGHGTLVTVSS

SEQ ID NO: 28 - protein, 12 aa, Artificial Sequence
  Synthetic construct
ESKYGPPCPPCP

SEQ ID NO: 29 - protein, 14 aa, Artificial Sequence
  Synthetic construct
VDESKYGPPCPPCP

SEQ ID NO: 30 - protein, 14 aa, Artificial Sequence
  Synthetic construct
VDESKYGPPCPPCP

SEQ ID NO: 31 - protein, 218 aa, Artificial Sequence
  Synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 32 - protein, 68 aa, Artificial Sequence
  Synthetic construct
FWVLVVVGGVLACYSLLVTVAFIIFWVRSKRSRGGHSDYMNMTPRRPGPTRKHYQPYAPPRDFAAYRS

SEQ ID NO: 33 - protein, 42 aa, Artificial Sequence
  Synthetic construct
KRGRKKLLYIFKQPFMRPVQTTQEEDGCSCRFPEEEEGGCEL

SEQ ID NO: 34 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - DNA, 2148 bp, Artificial Sequence
  Synthetic construct
atgggatggtcatgtatcatcctttttctagtagcaactgcaaccggtgtacattccgacatcaagatgacccagtctccatcttccatgtatgcatctctaggagagagagtcactatcacttgcaaggcgagtccggacattaatagctatttaagctggttccagcagaaaccagggaaatctcctaagaccctgatctatcgtgcaaacagattggttgatggggtcccatcaaggttcagtggcggtggatctgggcaagattattctctcaccatcaacagcctggagtatgaagatatgggaatttattattgtctacagtatgatgaatttccgtacacgttcggaggggggaccaagctggaaatgaaaggctccacctctggatccggcaagcccggatctggcgagggatccaccaagggcgaagtgaaactagtggagtctgggggaggcttagtgaagcctggagggtccctgaaactctcctgtgcagcctctggattcactttcagtagctatgccatgtcttgggttcgccagattccagagaagaggctggagtgggtcgcatccattagtcgtggtggtaccacctactatccagacagtgtgaagggccgattcaccatctccagagataatgtcaggaacatcctgtacctgcaaatgagcagtctgaggtctgaggacacggccatgtattactgtggaagatatgattacgacgggtactatgcaatggactactggggtcaaggaacctcagtcaccgtctcctcagagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtccctaggtaaaatgttttgggtgctggtggtggttggtggagtcctggcttgctatagcttgctagtaacagtggcctttattattttctgggtgaggagtaagaggagcaggggcggacacagtgactacatgaacatgactccccgccgccctgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 36 - DNA, 2154 bp, Artificial Sequence
  Synthetic construct
atgggatggtcatgtatcatcctttttctagtagcaactgcaaccggtgtacattccgacatccagatgacccagtctccatcctccctgtctgcatctgtaggagacagagtcaccatcacttgcaaggcgagtccggacattaatagctatttaagctggtttcagcagaggccaggccaatctccaaggcgcctaatttatcgtgcaaacagattggttgatggggtcccagacaggttcagtggcagtgggtcaggcactgatttcacactgaaaatcagcagggtggaggctgaggatgttggagtttattactgtctacagtatgatgaatttccgtacacgttcggccaagggaccaaggtggaaatcaaaggctccacctctggatccggcaagcccggatctggcgagggatccaccaagggcgaagtgcagctggtgcagtctggagcagaggtgaaaaagcccggggagtctctgaggatctcctgtaagggttctggattcactttcagtagctatgccatgtcttggatcaggcagtccccatcgagaggccttgagtggctgggttccattagtcgtggtggtaccacctactatccagacagtgtgaagggcagattcaccatctccagagacaacgccaagaactcactgtatctgcaaatgaacagcctgagagccgaggacacggctgtgtattactgtggaagatatgattacgacgggtactatgcaatggactactggggccagggaacgctggtcaccgtcagctcagtcgacgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtccctaggtaaaatgttttgggtgctggtggtggttggtggagtcctggcttgctatagcttgctagtaacagtggcctttattattttctgggtgaggagtaagaggagcaggggcggacacagtgactacatgaacatgactccccgccgccctgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 37 - DNA, 2175 bp, Artificial Sequence
  Synthetic construct
atgggatggtcatgtatcatcctttttctagtagcaactgcaaccggtgtacattccgatattgtgatgacccagactccactctccctgcccgtcacccctggagagccggcctccatctcctgcagggcaagtaagagcattagcaaatatttagcctggtaccagcagaaacctggccaggctcccaggctcctcatctattctggatccactttgcaatctgggatcccacctcgattcagtggcagcgggtatggaacagattttaccctcacaattaataacatagaatctgaggatgctgcatattacttctgtcaacagcatgatgaatccccgtacacgttcggcgaggggaccaaggtggaaatcaaaggtggtggtggtagcggctccacctctggatccggcaagcccggatctggcgagggatccaccaagggcggaggaggaggaagtcaggtgcagctgcaggagtcgggcccaggactggtgaagccttcacagaccctgtccctcacctgcactgtctctggttatgcattcactgcctacaacatacactgggtgcgacaggcccctggacaagggcttgagtggatgggttcttttgatccttacgatggtggtagtagttacaaccagaagttcaaggacagactcaccatctccaaggacacctccaaaaaccaggtggtccttacaatgaccaacatggaccctgtggacacagccacgtattactgtgcaagagggtggtactactttgactactggggccacggaaccctggtcaccgtctcctcagtcgacgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtccctaggtaaaatgttttgggtgctggtggtggttggtggagtcctggcttgctatagcttgctagtaacagtggcctttattattttctgggtgaggagtaagaggagcaggggcggacacagtgactacatgaacatgactccccgccgccctgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 38 - protein, 715 aa, Artificial Sequence
  Synthetic construct
MGWSCIILFLVATATGVHSDIKMTQSPSSMYASLGERVTITCKASPDINSYLSWFQQKPGKSPKTLIYRANRLVDGVPSRFSGGGSGQDYSLTINSLEYEDMGIYYCLQYDEFPYTFGGGTKLEMKGSTSGSGKPGSGEGSTKGEVKLVESGGGLVKPGGSLKLSCAASGFTFSSYAMSWVRQIPEKRLEWVASISRGGTTYYPDSVKGRFTISRDNVRNILYLQMSSLRSEDTAMYYCGRYDYDGYYAMDYWGQGTSVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 717 aa, Artificial Sequence
  Synthetic construct
MGWSCIILFLVATATGVHSDIQMTQSPSSLSASVGDRVTITCKASPDINSYLSWFQQRPGQSPRRLIYRANRLVDGVPDRFSGSGSGTDFTLKISRVEAEDVGVYYCLQYDEFPYTFGQGTKVEIKGSTSGSGKPGSGEGSTKGEVQLVQSGAEVKKPGESLRISCKGSGFTFSSYAMSWIRQSPSRGLEWLGSISRGGTTYYPDSVKGRFTISRDNAKNSLYLQMNSLRAEDTAVYYCGRYDYDGYYAMDYWGQGTLVTVSSVDESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 724 aa, Artificial Sequence
  Synthetic construct
MGWSCIILFLVATATGVHSDIVMTQTPLSLPVTPGEPASISCRASKSISKYLAWYQQKPGQAPRLLIYSGSTLQSGIPPRFSGSGYGTDFTLTINNIESEDAAYYFCQQHDESPYTFGEGTKVEIKGGGGSGSTSGSGKPGSGEGSTKGGGGGSQVQLQESGPGLVKPSQTLSLTCTVSGYAFTAYNIHWVRQAPGQGLEWMGSFDPYDGGSSYNQKFKDRLTISKDTSKNQVVLTMTNMDPVDTATYYCARGWYYFDYWGHGTLVTVSSVDESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 111 aa, Artificial Sequence
  Synthetic construct
VDESKYGPPCPPCPLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 231 aa, Artificial Sequence
  Synthetic construct
VDESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 43 - protein, 6 aa, Artificial Sequence
  Synthetic construct
KSISKY

SEQ ID NO: 44 - protein, 3 aa, Artificial Sequence
  Synthetic construct
SGS

SEQ ID NO: 45 - protein, 8 aa, Artificial Sequence
  Synthetic construct
QQHDESPY

SEQ ID NO: 46 - protein, 8 aa, Artificial Sequence
  Synthetic construct
GYAFTAYN

SEQ ID NO: 47 - protein, 8 aa, Artificial Sequence
  Synthetic construct
FDPYDGGS

SEQ ID NO: 48 - protein, 7 aa, Artificial Sequence
  Synthetic construct
GWYYFDY

SEQ ID NO: 49 - protein, 6 aa, Artificial Sequence
  Synthetic construct
PDINSY

SEQ ID NO: 50 - protein, 3 aa, Artificial Sequence
  Synthetic construct
RAN

SEQ ID NO: 51 - protein, 9 aa, Artificial Sequence
  Synthetic construct
LQYDEFPYT

SEQ ID NO: 52 - protein, 8 aa, Artificial Sequence
  Synthetic construct
GFTFSSYA

SEQ ID NO: 53 - protein, 7 aa, Artificial Sequence
  Synthetic construct
ISRGGTT

SEQ ID NO: 54 - protein, 11 aa, Artificial Sequence
  Synthetic construct
YDYDGYYAMDY

SEQ ID NO: 55 - protein, 937 aa, Homo sapiens
MHRPRRRGTRPPLLALLAALLLAARGAAAQETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILYILVPSVAIPLAIALLFFFICVCRNNQKSSSAPVQRQPKHVRGQNVEMSMLNAYKPKSKAKELPLSAVRFMEELGECAFGKIYKGHLYLPGMDHAQLVAIKTLKDYNNPQQWMEFQQEASLMAELHHPNIVCLLGAVTQEQPVCMLFEYINQGDLHEFLIMRSPHSDVGCSSDEDGTVKSSLDHGDFLHIAIQIAAGMEYLSSHFFVHKDLAARNILIGEQLHVKISDLGLSREIYSADYYRVQSKSLLPIRWMPPEAIMYGKFSSDSDIWSFGVVLWEIFSFGLQPYYGFSNQEVIEMVRKRQLLPCSEDCPPRMYSLMTECWNEIPSRRPRFKDIHVRLRSWEGLSSHTSSTTPSGGNATTQTTSLSASPVSNLSNPRYPNYMFPSQGITPQGQIAGFIGPPIPQNQRFIPINGYPIPPGYAAFPAAHYQPTGPPRVIQHCPPPKSRSPSSASGSTSTGHVTSLPSSGSNQEANIPLLPHMSIPNHPGGMGITVFGNKSQKPYKIDSKQASLLGDANIHGHTESMISAEL

SEQ ID NO: 56 - protein, 21 aa, Homo sapiens
VATNGKEVVSSTGVLFVKFGP

SEQ ID NO: 57 - protein, 15 aa, Homo sapiens
EVVSSTGVLFVKFGP

(19) United States
(12) Patent Application Publication
(10) Pub. No.: US 2024/0415883 A1
(43) Pub. Date: Dec. 19, 2024

(54) TREATMENT OF NEUROENDOCRINE CANCERS

(71) Applicant: The Regents of the University of California, Oakland, CA (US)

(72) Inventors: Charles E. Prussak, San Diego, CA (US); Christopher Oh, Porter Ranch, CA (US)

(21) Appl. No.: 18/274,145

(22) PCT Filed: Feb. 1, 2022
(86) PCT No.: PCT/US2022/014792

§ 371 Date: Jul. 25, 2023

Related U.S. Application Data
(60) Provisional application No. 63/144,337, filed on Feb. 1, 2021.

(51) Int. Cl. A61K 35/17; A61K 39/00; A61P 35/04; C07K 16/28

(52) U.S. Cl. CPC A61K 35/17; A61K 39/4611; A61K 39/4613; A61K 39/4631; A61K 39/464411; A61P 35/04; C07K 16/2803; A61K 2239/13; A61K 2239/17; A61K 2239/21; A61K 2239/22; A61K 2239/47

(57) ABSTRACT

Provided herein are compositions and methods for treating neuroendocrine cancer and preventing or treating metastasis originating from a neuroendocrine cancer.

### CROSS-REFERENCES TO RELATED APPLICATIONS

This application claims priority to U.S. Provisional Application No. 63/144,337, filed Feb. 1, 2021, which is hereby incorporated by reference in its entirety and for all purposes.

### SEQUENCE LISTING

The Sequence Listing written in file 048537-640001WO SEQUENCE LISTING ST25.TXT, created on Mar. 23, 2020, 69,632 bytes, machine format IBM-PC, MS-Windows operating system, is hereby incorporated by reference.

### BACKGROUND OF THE INVENTION

Neuroendocrine tumors (NET) are rare types of tumor that arise from neuroendocrine cells. These cells have traits of both nerve cells and hormone-producing cells, and release hormones into the blood in response to signals from the nervous system. Because neuroendocrine tumors arise from cells that produce hormones, the tumors can also produce hormones. Most neuroendocrine tumors occur in the digestive tract, pancreas, rectum, lungs, appendix, and prostate gland. Neuroendocrine tumors include, but are not limited to, carcinoid tumors, islet cell tumors, medullary thyroid cancer, pheochromocytomas, neuroendocrine carcinoma of the skin (Merkel cell cancer), small cell lung cancer, large cell neuroendocrine carcinoma, and neuroendocrine prostate cancer (NEPC).

NEPC is an aggressive histologic subtype of prostate cancer that most commonly arises in later stages of prostate cancer as treatment-resistant cancer. The poor prognosis of NEPC is attributed in part to late diagnosis and a lack of effective therapeutic agents. Prostate cancer is a disease regulated by androgen, and androgen deprivation therapy (ADT) in combination with drugs that target androgen receptor (AR) signaling is standard therapy for metastatic prostate cancer. Inevitably, however, the cancer becomes resistant to ADT, developing into lethal castration resistant prostate cancer (CRPC). CRPC is a lethal disease and the second leading cause of cancer death of men in the United States. One in six men will eventually be diagnosed with prostate cancer (PCa), making it a major health problem in the U.S. One quarter of the men diagnosed with this cancer, develop advanced PCa, which has a poor medical prognosis and greatly reduced relative five-year survival expectancies. Moreover, PCa often metastasizes to bone where it typically becomes resistant to therapy and is not curative at this stage of development. A particularly malignant form of CRPC called neuroendocrine prostate cancer (NEPC) is emerging with increasing frequency in patients treated with ADT. Despite the recent development of several novel therapies that have improved patient survival, nearly all patients with CRPC develop resistance to therapy and disease progression. Recent genome-wide next generation sequencing studies have identified recurrent molecular alterations in prostate cancer, elucidating mechanisms of treatment resistance and potential therapeutic vulnerabilities.

Prostate cancers may be targeted by CAR-T cells or other forms of immune therapy. Because there is early evidence of clinical activity of the CAR-T cells, many large multi-center clinical trials targeting either the prostate-specific membrane antigen (PMSA) (NCT04249947, NCT04227275) or the plasma membrane calcium ATPase (PSCA) (NCT04227275) are ongoing. However, there are aggressive CRPCs and NEPC that do not express PSMA or PSCA, and thus, there is an urgent unmet need to identify new prostate cancer antigens, which can be targeted by CAR T cells.

Tyrosine-protein kinase transmembrane receptor ROR1, also known as neurotrophic tyrosine kinase, receptor-related 1, is an enzyme that in humans is encoded by the ROR1 gene. ROR1 is a member of the receptor tyrosine kinase-like orphan receptor family. ROR1 is expressed on CRPC and NEPC, but not on normal post-natal and adult tissues. In addition, ROR1 appears to play a functional role in oncogenesis of PCa including the expansion of cancer specific stem-cells. Because it is expressed on the parental tumor and tumor and metastasis promoting stem-cells, ROR1 is an attractive therapeutic target.

WNT signaling is known to regulate many key cellular processes, and aberrant Wnt signaling and mutations in the pathway have been associated with tumorigenesis, progression, and metastasis in many cancers including prostate cancer (5, 6). Likewise, the deregulated expression of the WNT co-receptors ROR1 and ROR2 has been associated with several cellular features that promote malignancy, namely cell proliferation, survival, migration/invasion, and stemness. Wnt signaling was originally discovered as a group of signal transduction pathways critical for normal development and physiology (5, 6). Aberrant Wnt signaling, which is comprised of the canonical (β-catenin dependent) and noncanonical pathways, is frequently altered in prostate cancer. Comprehensive sequencing studies in patients with castration-resistant prostate cancer (CRPC) have identified recurrent molecular alterations in Wnt signaling pathway components in about 20% of advanced prostate cancer patients (4). Analysis of circulating tumor cells (CTCs) from CRPC patients demonstrate expression of Wnt5A, the prototypical noncanonical Wnt ligand, in >60% of patients with refractory disease (7, 8).

The Wnt signaling pathway is complex and context-dependent activities of Wnt signaling are mediated via crosstalk between the canonical and noncanonical Wnt signaling. The Wnt pathway interacts with androgen receptor (AR) signaling, a key pathway in prostate cancer pathogenesis (9). Noncanonical Wnt signaling is mediated in part through ROR1 tyrosine-kinase-like orphan receptor activation by Wnt5A ligand (FIG. 1). ROR1 is a conserved oncoembryonic surface protein expressed in prostate cancer cells (10). The expression of ROR1 can enhance epithelial-mesenchymal transition, cancer cell proliferation, migration and metastasis (11-13). Noncanonical Wnt signaling, mediated through ROR1, promotes bone metastases via induction of osteoblast activity (14). Moreover, ROR1 is overexpressed in chemoresistant tumors (15, 16). ROR1 expression is associated with adverse outcomes in patients, including poor therapy response and tumor recurrence (17-21). ROR1 regulates stability and transcription of the drug efflux pump ABCB1 which is overexpressed in chemoresistant tumors (22). ROR1 inhibition sensitizes cancer cells to chemotherapeutic agents and is directly correlated with decreased efflux of chemotherapeutic drugs from tumor cells (22). Furthermore, the interaction between ROR1 and cortactin plays an important role in cancer cell migration and metastasis (23, 24).

In preclinical models, Wnt5A stimulates and accelerates prostate cancer tumor growth, which is reduced by Wnt5A knockdown or haploinsufficiency (12). In addition to playing a role in tumorigenesis, noncanonical Wnt ligands have been shown to induce drug resistance to enzalutamide in vitro (25). Several clinical studies in advanced prostate cancer patients have shown that the noncanonical Wnt pathway is activated in advanced prostate cancer and correlates with poor prognosis in CRPC (8, 26-31). Whole transcriptome RNA-seq of single CTCs demonstrate significant enrichment for noncanonical Wnt signaling in patients with resistance to enzalutamide (7). Furthermore, Wnt5A expression in CTCs using multiplex qPCR found that Wnt5A was independently predictive of worse overall survival (8, 26).

To target this proto-oncogene, cirmtuzumab, a first-in-class ROR1 binding monoclonal antibody (mAb) was developed at the University of California San Diego (UCSD) (32). This human mAb has a high affinity for human ROR1 and no apparent off-target activities in in vitro or in vivo test systems. In a clinical study, the anti-ROR1 mAb was well tolerated with no antibody associated serious adverse events noted, a for a prolonged half-life suggesting limited or no off-tumor binding and early evidence of anti-tumor activity (NCT02222688)(33). Because of its favorable therapeutic index, cirmtuzumab has subsequently been employed in a number of clinical studies targeting lymphoid and solid tumor cancers in combination with standard chemotherapies (NCT02776917, 02860676 and 03088878), as the targeting moiety for an antibody drug conjugate (ADC) (NCT03833180) and clinical studies with this mAb are being planned for patients with CRPC.

Elevated expression of ROR1 have been demonstrated in highly malignant, taxane resistant, breast cancer cells. Because this mAb blocks ROR1 signaling by blocking Wnt5A mediated activation, cirmtuzumab has demonstrated substantial therapeutic activity against these chemotherapy resistant tumors in preclinical models. Based on these studies, a phase Ib clinical trial combining cirmtuzumab with paclitaxel is being tested in patients with advanced breast cancer (NCT02776917) (34). Like breast cancer, a taxane (docetaxel) based chemotherapy regimen is a mainstay for the treatment of patients with advanced CRPC (35). However, when combined with abiraterone and enzalutamide, this docetaxel containing chemotherapy cocktail has minimal activity in treating CPRC, generating a 3-month median time to progression and limited impact on reducing PSA levels (<30% of patients)(36). Additionally, noncanonical Wnt signaling has been hypothesized to be a mechanism of resistance to enzalutamide (7) and taxane chemotherapy (16). Available data suggest that Wnt blockade with chemotherapy may be the most effective way to implement Wnt pathway modulation (6).

A successful potent and specific therapy that targets treatment of progressive, malignant and lethal neuroendocrine tumors is urgently needed. The methods provided herein address these and other needs in the art.

### BRIEF SUMMARY OF THE INVENTION

Described herein, inter alia, are compositions and methods for treating a neuroendocrine cancer and methods of preventing or treating metastasis originating from a neuroendocrine cancer, that comprise the administration of cells that express chimeric antigen receptors that target human ROR-1. The chimeric antigen receptors described herein may be expressed by T lymphocytes or natural killer cells isolated from an individual afflicted with neuroendocrine cancer and re-administered to the individual. Administration of T cells expressing the CARs described herein may serve as an effective therapeutic treatment for neuroendocrine cancers that express ROR-1. The function and efficacy of anti-ROR-1 CAR-T cells may be improved by optimized intracellular signaling domains and membrane spacers, as described herein.

In aspects, the chimeric antigen receptors comprise i) an antigen binding region, wherein the antigen binding region specifically binds ROR-1 and wherein the antigen binding region comprises a light chain variable domain and a heavy chain variable domain, ii) a spacer domain, iii) a transmembrane domain, and iv) an intracellular domain. The light chain variable domain comprises a CDR L1 as set forth in SEQ ID NO:43, a CDR L2 as set forth in SEQ ID NO:44 and a CDR L3 as set forth in SEQ ID NO:45, and the heavy chain variable domain comprises a CDR H1 as set forth in SEQ ID NO:46, a CDR H2 as set forth in SEQ ID NO:47, and a CDR H3 as set forth in SEQ ID NO:48. Alternatively, the light chain variable domain comprises a CDR L1 as set forth in SEQ ID NO:49, a CDR L2 as set forth in SEQ ID NO:50 and a CDR L3 as set forth in SEQ ID NO:51, and the heavy chain variable domain comprises a CDR H1 as set forth in SEQ ID NO:52, a CDR H2 as set forth in SEQ ID NO:53, and a CDR H3 as set forth in SEQ ID NO:54.

In embodiments, the light chain variable domain is covalently linked to the heavy chain variable domain through a polypeptide linker. In embodiments, the polypeptide linker comprises an amino acid sequence of SEQ ID NO: 24.

In embodiments, the spacer domain comprises an antibody domain. In embodiments, the antibody domain comprises an immunoglobulin hinge domain, an immunoglobulin constant heavy chain 3 (CH3) domain, an immunoglobulin constant heavy chain 2 (CH2) domain, or a combination thereof.

In embodiments, the spacer domain comprises an amino acid sequence represented by SEQ ID NO: 29, SEQ ID NO: 41 or SEQ ID NO: 42.

In embodiments, the light chain variable domain comprises an amino acid sequence at least about 90%, 95%, 97%, 98%, 99%, or 100% identical to SEQ ID NO: 21.

In embodiments, the heavy chain variable domain comprises an amino acid sequence at least about 90%, 95%, 97%, 98%, 99%, or 100% identical to SEQ ID NO: 27.

In embodiments, the transmembrane domain comprises a CD8α transmembrane domain, a CD28 transmembrane domain, a CD4 transmembrane domain, a CD3ζ transmembrane domain, or any combination thereof. In embodiments, the transmembrane domain is a CD28 transmembrane domain.

In embodiments, the intracellular domain comprises an intracellular co-stimulatory signaling domain, an intracellular T-cell signaling domain, or a combination thereof. The intracellular co-stimulatory signaling domain is a 4-1BB intracellular co-stimulatory signaling domain, a CD28 intracellular co-stimulatory signaling domain, a ICOS intracellular co-stimulatory signaling domain, an OX-40 intracellular co-stimulatory signaling domain, or any combination thereof. In embodiments, the intracellular costimulatory signaling domain comprises a 41-BB intracellular co-stimulatory signaling domain. In embodiments, the intracellular costimulatory signaling domain comprises a CD28 intracellular co-stimulatory signaling domain and a 41-BB intracellular co-stimulatory signaling domain.

In embodiments, the intracellular costimulatory signaling domain further comprises an intracellular T-cell signaling domain. In embodiments, the intracellular T-cell signaling domain is a CD3ζ intracellular T-cell signaling domain.

In embodiments, the chimeric antigen receptor binds to a cell expressing ROR-1. In embodiments, the cell expressing ROR-1 is a neuroendocrine cancerous cell.

In embodiments, the cell is a T lymphocyte. In embodiments, the T lymphocyte is a CD4+T lymphocyte or a CD8+T lymphocyte.

In embodiments, the cell is a natural killer cell. In embodiments, the natural killer cell is autologous to the subject. In embodiments, the natural killer cell is heterologous to the subject. In embodiments, the natural killer cell is allogeneic to the subject.

Aspects disclosed herein provide a nucleic acid encoding the chimeric antigen receptor described herein. In embodiments, the nucleic acid is a viral vector. In embodiments, the viral vector is a lentiviral vector.

Aspects disclosed herein provide a cell comprising the nucleic acid described herein. Aspects disclosed herein also provide a cell expressing the chimeric antigen receptor described herein. In embodiments, the cell is a T lymphocyte. In embodiments, the T lymphocyte is a CD4+T lymphocyte or a CD8+T lymphocyte. In embodiments, the cell is a natural killer cell, a genetically engineered natural killer cell or a CD56+ cell. In embodiments, the cell is a natural killer cell. In embodiments, the natural killer cell is autologous to the subject. In embodiments, the natural killer cell is heterologous to the subject. In embodiments, the natural killer cell is allogeneic to the subject.

Aspects disclosed herein provide a pharmaceutical composition comprising a therapeutically effective amount of the cells described herein and a pharmaceutically acceptable diluent, carrier, or excipient. In embodiments, the composition is formulated for intravenous injection.

Aspects disclosed herein provide methods of treating a neuroendocrine cancer in an subject in need thereof, and methods of preventing or treating a metastasis in a subject with a neuroendocrine cancer, which comprise administering to the subjects the cells as described herein, and pharmaceutical compositions comprising the cells provided herein and excipients, additives and the like. In embodiments, the neuroendocrine cancer is a carcinoid tumor, an islet cell tumor, a medullary thyroid cancer, a pheochromocytoma, a neuroendocrine carcinoma of the skin, small cell lung cancer, large cell neuroendocrine carcinoma, neuroendocrine prostate cancer (NEPC), or metastatic castration-resistant prostate cancer (CRPC). In embodiments, the neuroendocrine cancer is metastatic castration-resistant prostate cancer (CRPC).

In embodiments, the neuroendocrine cancerous cells express ROR-1. In embodiments, the subject has failed to respond to androgen deprivation therapy. In embodiments, the neuroendocrine cancer has metastasized to bone.

In embodiments, the methods provided herein further comprise administering cirmtuzumab to the subject. In embodiments, the cirmtuzumab and the cells expressing the disclosed chimeric antigen receptors are administered separately. In embodiments, the cirmtuzumab and the cells are administered together.

In embodiments, the methods provided herein further comprise administering to the subject platinum-based chemotherapy. In embodiments, the platinum-based chemotherapy comprises carboplatin, cisplatin, etoposide, a taxane, or any combination thereof. In embodiments, the platinum-based chemotherapy comprises administering to the subject an antibody-drug conjugate.

### BRIEF DESCRIPTION OF THE DRAWINGS

FIG. 1 is a schematic representation showing signaling through the non-canonical Wnt pathway is mediated by Wnt5A binding to its receptor ROR1 and ROR2. Cirmtuzumab is a monoclonal antibody which targets ROR1.

FIG. 2 is a schematic representation showing a Cirmtuzumab-based Chimeric Antigen Receptor (CAR) T cell targeting ROR-1.

FIG. 3 present graphs showing in vitro cell killing activity of ROR1 CAR T-cells in a 4 h chromium release assay (left panel) and 120 h ACEA impedance assay (right panel) from T cells of two healthy donors, that were transduced with ROR1 at the indicated effect to target (E:T) ratios against leukemic Mec ROR1pos cells in the left panel and an ACEA impedance assay against MB 231 ROR1pos breast cancer cells in the right panel. The anti-ROR1 CAR T-cells demonstrated high and specific cytotoxicity without significant killing of ROR1-negative target cells.

FIG. 4 shows bioluminescence imaging of mice inoculated with MEC1-ROR1 cells and with ROR1 CAR T-Cells. Animals treated with CAR-T cells had reduced disease burden compared to controls. The highest dose (3×10⁶ CAR-T cells) cohort showed reduction of the leukemic burden to background levels by day 30, and had only minimal amounts of disease for the duration of the study. Animals in the control groups (untreated, mock transduced) had to be sacrificed on day 20. The right panel shows the total bioluminescent product collected from the mice. Blue square and circle are controls and green triangles ROR1-CAR T treated mice.

FIG. 5 shows ROR-1 CAR T cell expression in mouse tissues at various times. Following ROR-1 CAR-T administration, animals were sacrificed on days 11 (top panels) and day 25 (bottom panels). Blood and organs were collected and subjected to flow analysis for CAR expression and confirmatory ROR1 binding activity. The ROR-1 CAR-T cell number was substantially greater in mice bearing MEC-1 ROR1 cells (CAR+MEC1-ROR-1) vs control (CAR only), demonstrating elevated expansion of ROR-1 CAR-T cells in animals with tumor burden. Bars represent the mean values from the five mice in each group and error bars represent the S.E of the mean.

FIG. 6 shows the results obtained from the analysis of tumor RNA sequences obtained from 66 prostate cancer samples, expressed as association of single-sample Gene Set Enrichment Analysis profiles of non-canonical WNT and stem cell gene sets with the expression of ROR1 (mRNA). The analysis shows a significant enrichment of WNT non-canonical and stem cell gene sets against ROR1 mRNA expression.

FIGS. 7A-7C present xenograft models obtained from prostate cancer patients. FIG. 7A shows hierarchical cluster of RNA-seq analysis on PCSD1 and PCSD13. FIG. 7B shows flow cytometry (FACS) of ROR-1 protein on the cell surface of PCSD13 cells. FIG. 7C shows that ROR1 is expressed on the cell surface of prostate cancer cell lines. Antibody Isotype controls are in gray. Negative control cell line: Breast cancer line, MCF7, and ROR-1 over-expressing line, MCF7_ROR1. Prostate Cancer lines: PC3 and DU145.

FIG. 8 shows PCSD1 PDX tumors cells from three-dimensional organoids that replicate histomorphology of the xenografts growing in the femur bone. Three-dimensional organoid cell cultures from subjects with prostate cancer were established. The 3D cultures consisted of two main cell masses: spheroids and epithelial cysts similar to gland-like structures seen in sections from xenografts growing within the femur bone displacing bone marrow and in the patient. Spheroids were resistant to androgen deprivation-induced death. Spheroid masses contained cyst-like structures surrounded by tumor cells that were similar to gland-like structures. Black Arrows point to the cyst structures. Organoids are GFP+. PDXs were obtained from prostate cancer bone metastases. PCSD1 expressing GFP-Luciferase formed different morphologies under 3D-culture conditions. Enzalutamide treatment of PCSD1 reduced lumen size and number of epithelial cysts. Transcriptome analysis showed that AR-responsive genes such as PSA (KLK3) and TMPRSS2 were downregulated under ADT while stem-cell transcription factors, steroidogenic and neurogenic pathway genes were upregulated. Thus, anti-androgens could still suppress canonical AR-responsive genes, but the tumor and organoid growth were resistant to ADT including the anti-androgen, enzalutamide.

FIG. 9 presents graphs showing that ROR1 CAR-T cells kill ROR1 expressing prostate cancer cell lines. ROR1 CAR-T cells showed significant cytotoxic killing of PCa cell lines, PC3 or DU145 cells, in culture. Increasing effect (E=T cell) to Target (T=PCa cell) ratio showed increased killing compared to Control T cells in Incucyte assay. CAR-Ts cytotoxic T cell killing is Effect: Target (E:T) dose-dependent.

FIG. 10A-10B presents graphs showing that anti-ROR1 CAR-T cells specifically killed PDX pCSD13 cells in Effector:Target (E:T) dose-dependent manner in culture in Incucyte Cytotoxicity assay. Left panel (A): control T cells plus PCSD13 (Target) cells freshly isolated from xenograft and cultured for 48 hours. Right panel (B): anti-ROR1 CAR-T (Effectors). The highest dose of anti-ROR1 CAR-T cells completely killed the PCSD13 cells (Blue line, E:T 3) compared to no CAR-Ts (black line E:T 0).

FIG. 11 shows bioluminescence imaging of mice implanted subcutaneously with PC3 PCa cells and treated with ROR1 CAR-T cells. Animals were implanted with GFP luciferase expressing PC3 cells on day 1 and were treated with a one-time injection of 3e7 CAR-T cells IV (CAR IV) or activated mock transduced cell (Control) or 1e7 cells administered intra-tumorly (CAR IT) As shown, the CAR-T treated mice that received a single intravenous or intra-tumoral injection had reduced disease burden when compared to control animals, which had to be sacrificed by week 5. The CAR-T IV treated cohort had only minimal amounts of disease at the end of the study.

FIG. 12 is a graph showing tumor Volume Measurement of Implanted PC3 cells by Caliper Measurement. Tumor volumes were measured by weekly caliper measurement for mice treated as described in FIG. 11. Results are consistent with the bioluminescent measurements and better demonstrate the activity of the intravenously administered CAR product. Tumor Volume was determined with the formula (mm³)=(L×w²)/2 The value w (width) is the smaller of two perpendicular tumor axes and the value L (length) is the larger of two perpendicular axes. Error bars represent the SO of the mean for the 3 measurements.

FIG. 13 shows optimized RIC staining analysis of ROR1 protein expression patterns in formalin-fixed paraffin-embedded (FFPE) breast adenocarcinoma tissue as compared to a negative control.

### DETAILED DESCRIPTION OF THE INVENTION

### Definitions

While various embodiments and aspects of the present invention are shown and described herein, it will be understood to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., Dictionary Of Microbiology And Molecular Biology 2nd ed., J. Wiley & Sons (New York, NY 1994); Sambrook et al., Molecular Cloning, A Laboratory Manual, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acid as used herein also refers to nucleic acids that have the same basic chemical structure as a naturally occurring nucleic acid. Such analogues have modified sugars and/or modified ring substituents, but retain the same basic chemical structure as the naturally occurring nucleic acid. A nucleic acid mimetic refers to chemical compounds that have a structure that is different from the general chemical structure of a nucleic acid, but that functions in a manner similar to a naturally occurring nucleic acid. Examples of such analogues include, without limitation, phosphorothiolates, phosphoramidates, methyl phosphonates, chiral-methyl phosphonates, 2-O-methyl ribonucleotides, and peptide-nucleic acids (PNAs).

The term “gene” means the segment of DNA involved in producing a protein; it includes regions preceding and following the coding region (leader and trailer) as well as intervening sequences (introns) between individual coding segments (exons). The leader, the trailer, as well as the introns, include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules may be detected by standard PCR or Northern blot methods well known in the art. See, Sambrook et al., 1989 Molecular Cloning: A Laboratory Manual, 18.1-18.88.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The terms “transfection”, “transduction”, “transfecting” or “transducing” are used interchangeably throughout and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection, and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

The term “plasmid” or “expression vector” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “numbered with reference to” or “corresponding to,” when used in the context of the numbering of a given amino acid or polynucleotide sequence, refer to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., ROR-1) in other proteins with different numbering systems. For example, by performing a simple sequence alignment with a protein (e.g., ROR-1) the identity and location of residues corresponding to specific positions of the protein are identified in other protein sequences aligning to the protein. For example, a selected residue in a selected protein corresponds to glutamic acid at position 138 when the selected residue occupies the same essential spatial or other structural relationship as a glutamic acid at position 138. In some embodiments, where a selected protein is aligned for maximum homology with a protein, the position in the aligned selected protein aligning with glutamic acid 138 is the to correspond to glutamic acid 138. Instead of a primary sequence alignment, a three dimensional structural alignment can also be used, e.g., where the structure of the selected protein is aligned for maximum correspondence with the glutamic acid at position 138, and the overall structures compared. In this case, an amino acid that occupies the same essential position as glutamic acid 138 in the structural model is the glutamic acid 138 residue.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

The term “recombinant” when used with reference, for example, to a cell, a nucleic acid, a protein, or a vector, indicates that the cell, nucleic acid, protein or vector has been modified by or is the result of laboratory methods. Thus, for example, recombinant proteins include proteins produced by laboratory methods. Recombinant proteins can include amino acid residues not found within the native (non-recombinant) form of the protein or can be include amino acid residues that have been modified (e.g., labeled).

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then “substantially identical.” This definition also refers to the complement of a test sequence. Optionally, the identity exists over a region that is at least about 50 nucleotides in length, or more preferably over a region that is 100 to 500 or 1000 or more nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Any methods of alignment of sequences for comparison well known in the art are contemplated. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

Example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross-reactive with the antibodies raised against the polypeptide encoded by the second nucleic acid, as described below. Thus, a polypeptide is typically or substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An “antibody variant” as provided herein refers to a polypeptide capable of binding to an antigen and including one or more structural domains (e.g., light chain variable domain, heavy chain variable domain) of an antibody or fragment thereof. Non-limiting examples of antibody variants include single-domain antibodies or nanobodies, monospecific Fab2, bispecific Fab2, trispecific Fab3, monovalent IgGs, scFv, bispecific antibodies, bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The terms “CDR L1”, “CDR L2” and “CDR L3” as provided herein refer to the complementarity determining regions (CDR) 1, 2, and 3 of the variable light (L) chain of an antibody. In embodiments, the variable light chain provided herein includes in N-terminal to C-terminal direction a CDR L1, a CDR L2 and a CDR L3. Likewise, the terms “CDR Hi”, “CDR H2” and “CDR H3” as provided herein refer to the complementarity determining regions (CDR) 1, 2, and 3 of the variable heavy (H) chain of an antibody. In embodiments, the variable light chain provided herein includes in N-terminal to C-terminal direction a CDR L1, a CDR L2 and a CDR L3.

The term “antibody” is used according to its commonly known meaning in the art.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

The term “antigen” as provided herein refers to molecules capable of binding to the antibody binding domain provided herein. An “antigen binding domain” as provided herein is a region of an antibody that binds to an antigen (epitope). As described above, the antigen binding domain is generally composed of one constant and one variable domain of each of the heavy and the light chain (VL, VH, CL and CHI, respectively). The paratope or antigen-binding site is formed on the N-terminus of the antigen binding domain. The two variable domains of an antigen binding domain typically bind the epitope on an antigen.

Antibodies exist, for example, as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially the antigen binding portion with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., e.g., single chain Fv) or those identified using phage display libraries (see, e.g., e.g., McCafferty et al., Nature 348:552-554 (1990)).

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The term “polypeptide linker” alternatively referred to as “polypeptide sequence” refers to a polypeptide segment that covalently links two adjacent domains within a protein. “Polypeptide linker” or “polypeptide sequence” as used herein covalently couple the light chain variable domain to the heavy chain variable domain. The linker can either connect the N-terminus of the V_{H} with the C-terminus of the V_{L}, or vice versa.

Polypeptide linkers may range from about 20 to about 60 amino acids in length. Short linker peptides may comprise from about 10 to about 25 amino acids. In embodiments, the polypeptide linker comprises between 10 and 50 amino acids. In embodiments, the polypeptide linker comprises between 10 and 40 amino acids. In embodiments, the polypeptide linker comprises between 10 and 30 amino acids. In embodiments, the polypeptide linker comprises between 10 and 20 amino acids. In embodiments, the polypeptide linker comprises between 10 and 15 amino acids. The linker may usually be rich in glycine for flexibility, as well as serine or threonine for solubility.

The epitope of an antibody is the region of its antigen to which the antibody binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

The term “ROR-1” or “ROR1” as used herein refers to any of the recombinant or naturally-occurring forms of tyrosine kinase-like orphan receptor 1 (ROR-1) or variants or homologs thereof that maintain ROR-1 activity (e.g., within at least 50%, 80%, 90%, 95%, 96%, 97%, 98%, 99% or 100% activity compared to ROR-1). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring ROR-1 protein. In embodiments, the ROR-1 protein is substantially identical to the protein identified by Accession No. NP_005003.1 or a variant or homolog having substantial identity thereto. In embodiments, the ROR-1 protein includes the amino acid sequence of SEQ ID NO:55. In embodiments, the ROR-1 protein is the amino acid sequence of SEQ ID NO: 55. In embodiments, the ROR-1 protein includes the amino acid sequence of SEQ ID NO:56. In embodiments, the ROR-1 protein includes the amino acid sequence of SEQ ID NO:57.

The terms “cirmtuzumab”, “UC-961”, and “99961.1” refer to a humanized monoclonal antibody capable of binding the extracellular domain of the human receptor tyrosine kinase-like orphan receptor 1 (ROR-1). In embodiments, cirmtuzumab is any one of the antibodies or fragments thereof disclosed in U.S. patent application Ser. No. 14/422,519, which is incorporated by reference herein in its entirety and for all purposes.

The term “CD28 transmembrane domain” as provided herein includes any of the recombinant or naturally-occurring forms of the transmembrane domain of CD28, or variants or homologs thereof that maintain CD28 transmembrane activity (e.g. within at least 50%, 80%, 90%, 95%, 96%, 97%, 98%, 99% or 100% activity compared to the CD28 transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD28 transmembrane domain polypeptide. In embodiments, the CD28 transmembrane domain is a human CD28 transmembrane domain protein. In embodiments, a variant or mutant of the CD28 transmembrane domain protein includes no more than 5, 4, 3, 2, or 1 deletions compared to the naturally occurring CD28 transmembrane domain protein. In embodiments, a variant or mutant of the CD28 transmembrane domain protein includes no more than 5, 4, 3, 2, or 1 insertions compared to the naturally occurring CD28 transmembrane domain protein. In embodiments, a variant or mutant of the CD28 transmembrane domain protein does not include deletions compared to the naturally occurring CD28 transmembrane domain protein. In embodiments, a variant or mutant of the CD28 transmembrane domain protein does not include insertions compared to the naturally occurring CD28 transmembrane domain protein. In embodiments, a variant or mutant of the CD28 transmembrane domain protein includes substitutions that are conservative substitutions compared to the naturally occurring CD28 transmembrane domain protein. In embodiments, the CD28 transmembrane domain includes all or a portion of the protein as identified by NCBI sequence reference NP_001230006.1, or an isoform or naturally occurring mutant or variant thereof. In embodiments, the CD28 transmembrane domain includes all or a portions of the protein as identified by the NCBI sequence reference NP_001230007.1, or an isoform or naturally occurring mutant or variant thereof. In embodiments, the CD28 transmembrane domain includes all or a portion of the protein as identified by the NCBI sequence reference NP_006130.1, or an isoform or naturally occurring mutant or variant thereof.

The term “CD4 transmembrane domain” as provided herein includes any of the recombinant or naturally-occurring forms of the transmembrane domain of CD4, or variants or homologs thereof that maintain CD4 transmembrane domain activity (e.g., within at least 50%, 80%, 90%, 95%, 96%, 97%, 98%, 99% or 100% activity compared to the CD4 transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD4 transmembrane domain polypeptide. In embodiments, the CD4 transmembrane domain is a human CD4 transmembrane domain protein. In embodiments, a variant or mutant of the CD4 transmembrane domain protein includes no more than 5, 4, 3, 2, or 1 deletions compared to the naturally occurring CD4 transmembrane domain protein. In embodiments, a variant or mutant of the CD4 transmembrane domain protein includes no more than 5, 4, 3, 2, or 1 insertions compared to the naturally occurring CD4 transmembrane domain protein. In embodiments, a variant or mutant of the CD4 transmembrane domain protein does not include deletions compared to the naturally occurring CD4 transmembrane domain protein. In embodiments, a variant or mutant of the CD4 transmembrane domain protein does not include insertions compared to the naturally occurring CD4 transmembrane domain protein. In embodiments, a variant or mutant of the CD4 transmembrane domain protein includes substitutions that are conservative substitutions compared to the naturally occurring CD4 transmembrane domain protein. In embodiments, the CD4 transmembrane domain includes all or a portion of the protein identified by the NCBI sequence reference NP_000607.1, or an isoform or naturally occurring mutant or variant thereof. In embodiments, the CD4 transmembrane domain includes all or a portion of the protein identified by the NCBI sequence reference NP_001181943.1, or an isoform or naturally occurring mutant or variant thereof. In embodiments, the CD4 transmembrane domain includes all or a portion of the protein identified by the NCBI sequence reference NP_001181944.1, or an isoform or naturally occurring mutant or variant thereof. In embodiments, the CD4 transmembrane domain includes all or a portion of the protein identified by the NCBI sequence reference NP_001181945.1, or an isoform or naturally occurring mutant or variant thereof. In embodiments, the CD4 transmembrane domain includes all or a portion of the protein identified by the NCBI sequence reference NP_001181946.1, or an isoform or naturally occurring mutant or variant thereof.

The term “CD8 transmembrane domain” as provided herein includes any of the recombinant or naturally-occurring forms of the transmembrane domain of CD8, or variants or homologs thereof that maintain CD8 transmembrane domain activity (e.g., within at least 50%, 80%, 90%, 95%, 96%, 97%, 98%, 99% or 100% activity compared to the CD8 transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD8 transmembrane domain polypeptide. In embodiments, the CD8 transmembrane domain is CD8A transmembrane domain. In embodiments, the CD8 transmembrane domain is a CD8B transmembrane domain. In embodiments, the CD8 transmembrane domain is a human CD8 transmembrane domain protein. In embodiments, a variant or mutant of the CD8 transmembrane domain protein includes no more than 5, 4, 3, 2, or 1 deletions compared to the naturally occurring CD8 transmembrane domain protein. In embodiments, a variant or mutant of the CD8 transmembrane domain protein includes no more than 5, 4, 3, 2, or 1 insertions compared to the naturally occurring CD8 transmembrane domain protein. In embodiments, a variant or mutant of the CD8 transmembrane domain protein does not include deletions compared to the naturally occurring CD8 transmembrane domain protein. In embodiments, a variant or mutant of the CD8 transmembrane domain protein does not include insertions compared to the naturally occurring CD8 transmembrane domain protein. In embodiments, a variant or mutant of the CD8 transmembrane domain protein includes substitutions that are conservative substitutions compared to the naturally occurring CD8 transmembrane domain protein. In embodiments, the CD8 transmembrane domain includes all or a portion of the protein identified by the NCBI sequence reference NP_001139345.1, or an isoform or naturally occurring mutant or variant thereof. In embodiments, the CD8 transmembrane domain includes all or a portion of the protein identified by the NCBI sequence reference NP_001181943.1, or an isoform or naturally occurring mutant or variant thereof. In embodiments, the CD8 transmembrane domain includes all or a portion of the protein identified by the NCBI sequence reference NP_001181944.1, or an isoform or naturally occurring mutant or variant thereof. In embodiments, the CD8 transmembrane domain includes all or a portion of the protein identified by the NCBI sequence reference NP_001181945.1, or an isoform or naturally occurring mutant or variant thereof. In embodiments, the CD8 transmembrane domain includes all or a portion of the protein identified by the NCBI sequence reference NP_741969.1, or an isoform or naturally occurring mutant or variant thereof. In embodiments, the CD8 transmembrane domain includes all or a portion of the protein identified by the NCBI sequence reference NP_001759.3, or an isoform or naturally occurring mutant or variant thereof. In embodiments, the CD8 transmembrane domain includes all or a portion of the protein identified by the NCBI sequence reference XP_011531466.1, or an isoform or naturally occurring mutant or variant thereof. In embodiments, the CD8 transmembrane domain includes all or a portion of the protein identified by the NCBI sequence reference NP_001171571.1, or an isoform or naturally occurring mutant or variant thereof. In embodiments, the CD8 transmembrane domain includes all or a portion of the protein identified by the NCBI sequence reference NP_757362.1, or an isoform or naturally occurring mutant or variant thereof. In embodiments, the CD8 transmembrane domain includes all or a portion of the protein identified by the NCBI sequence reference NP_742100.1, or an isoform or naturally occurring mutant or variant thereof. In embodiments, the CD8 transmembrane domain includes all or a portion of the protein identified by the NCBI sequence reference NP_742099.1, or an isoform or naturally occurring mutant or variant thereof. In embodiments, the CD8 transmembrane domain includes all or a portion of the protein identified by the NCBI sequence reference NP_004922.1, or an isoform or naturally occurring mutant or variant thereof.

The term “CD3-zeta transmembrane domain” as provided herein includes any of the recombinant or naturally-occurring forms of the transmembrane domain of CD3-zeta, or variants or homologs thereof that maintain CD3-zeta transmembrane domain activity (e.g., within at least 50%, 80%, 90%, 95%, 96%, 97%, 98%, 99% or 100% activity compared to the CD3-zeta transmembrane domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD3-zeta transmembrane domain polypeptide. In embodiments, the CD3-zeta transmembrane domain is a human CD3-zeta transmembrane domain protein. In embodiments, a variant or mutant of the CD3-zeta transmembrane domain protein includes no more than 5, 4, 3, 2, or 1 deletions compared to the naturally occurring CD3-zeta transmembrane domain protein. In embodiments, a variant or mutant of the CD3-zeta transmembrane domain protein includes no more than 5, 4, 3, 2, or 1 insertions compared to the naturally occurring CD3-zeta transmembrane domain protein. In embodiments, a variant or mutant of the CD3-zeta transmembrane domain protein does not include deletions compared to the naturally occurring CD3-zeta transmembrane domain protein. In embodiments, a variant or mutant of the CD3-zeta transmembrane domain protein does not include insertions compared to the naturally occurring CD3-zeta transmembrane domain protein. In embodiments, a variant or mutant of the CD3-zeta transmembrane domain protein includes substitutions that are conservative substitutions compared to the naturally occurring CD3-zeta transmembrane domain protein. In embodiments, the CD3-zeta transmembrane domain includes all or a portion of the protein identified by the NCBI sequence reference NP_000725.1, or an isoform or naturally occurring mutant or variant thereof. In embodiments, the CD3-zeta transmembrane domain includes all or a portion of the protein identified by the NCBI sequence reference NP_932170.1, or an isoform or naturally occurring mutant or variant thereof.

The term “CD28 co-stimulatory domain” as provided herein includes any of the recombinant or naturally-occurring forms of the co-stimulatory domain of CD28, or variants or homologs thereof that maintain CD28 co-stimulatory domain activity (e.g. within at least 50%, 80%, 90%, 95%, 96%, 97%, 98%, 99% or 100% activity compared to the CD28 co-stimulatory domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD28 co-stimulatory domain polypeptide. In embodiments, the CD28 co-stimulatory domain is a human CD28 co-stimulatory domain protein. In embodiments, a variant or mutant of the CD28 co-stimulatory domain protein includes no more than 5, 4, 3, 2, or 1 deletions compared to the naturally occurring CD28 co-stimulatory domain protein. In embodiments, a variant or mutant of the CD28 co-stimulatory domain protein includes no more than 5, 4, 3, 2, or 1 insertions compared to the naturally occurring CD28 co-stimulatory domain protein. In embodiments, a variant or mutant of the CD28 co-stimulatory domain protein does not include deletions compared to the naturally occurring CD28 co-stimulatory domain protein. In embodiments, a variant or mutant of the CD28 co-stimulatory domain protein does not include insertions compared to the naturally occurring CD28 co-stimulatory domain protein. In embodiments, a variant or mutant of the CD28 co-stimulatory domain protein includes substitutions that are conservative substitutions compared to the naturally occurring CD28 co-stimulatory domain protein. In embodiments, the CD28 co-stimulatory domain includes all or a portion of the protein identified by the NCBI sequence reference NP_001230006.1, or an isoform or naturally occurring mutant or variant thereof. In embodiments, the CD28 co-stimulatory domain includes all or a portion of the protein identified by the NCBI sequence reference NP_001230007.1, or an isoform or naturally occurring mutant or variant thereof. In embodiments, the CD28 co-stimulatory domain includes all or a portion of the protein identified by the NCBI sequence reference NP_006130.1, or an isoform or naturally occurring mutant or variant thereof.

The term “4-1BB co-stimulatory domain” as provided herein includes any of the recombinant or naturally-occurring forms of the co-stimulatory domain of 4-1BB, or variants or homologs thereof that maintain 4-1BB co-stimulatory domain activity (e.g., within at least 50%, 80%, 90%, 95%, 96%, 97%, 98%, 99% or 100% activity compared to the 4-1BB co-stimulatory domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring 4-1BB co-stimulatory domain polypeptide. In embodiments, the 4-1BB co-stimulatory domain is a human 4-1BB co-stimulatory domain protein. In embodiments, a variant or mutant of the 4-1BB co-stimulatory domain protein includes no more than 5, 4, 3, 2, or 1 deletions compared to the naturally occurring 4-1BB co-stimulatory domain protein. In embodiments, a variant or mutant of the 4-1BB co-stimulatory domain protein includes no more than 5, 4, 3, 2, or 1 insertions compared to the naturally occurring 4-1BB co-stimulatory domain protein. In embodiments, a variant or mutant of the 4-1BB co-stimulatory domain protein does not include deletions compared to the naturally occurring 4-1BB co-stimulatory domain protein. In embodiments, a variant or mutant of the 4-1BB co-stimulatory domain protein does not include insertions compared to the naturally occurring 4-1BB co-stimulatory domain protein. In embodiments, a variant or mutant of the 4-1BB co-stimulatory domain protein includes substitutions that are conservative substitutions compared to the naturally occurring 4-1BB co-stimulatory domain protein. In embodiments, the 4-1BB co-stimulatory domain protein amino acid sequence has at least or about 50%, 60%, 70%, 75%, 80%, 85%, 90%, 91%, 92%, 93%, 94%, 95%, 96%, 97%, 98%, or 99% sequence identify to SEQ ID NO:14. In embodiments, the 4-1BB co-stimulatory domain includes all or a portion of the protein identified by the NCBI sequence reference NP_001552.2 or an isoform or naturally occurring mutant or variant thereof.

The term “ICOS co-stimulatory domain” as provided herein includes any of the recombinant or naturally-occurring forms of the co-stimulatory domain of ICOS, or variants or homologs thereof that maintain ICOS co-stimulatory domain activity (e.g., within at least 50%, 80%, 90%, 95%, 96%, 97%, 98%, 99% or 100% activity compared to the ICOS co-stimulatory domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ICOS co-stimulatory domain polypeptide. In embodiments, the ICOS co-stimulatory domain is a human ICOS co-stimulatory domain protein. In embodiments, a variant or mutant of the ICOS co-stimulatory domain protein includes no more than 5, 4, 3, 2, or 1 deletions compared to the naturally occurring ICOS co-stimulatory domain protein. In embodiments, a variant or mutant of the ICOS co-stimulatory domain protein includes no more than 5, 4, 3, 2, or 1 insertions compared to the naturally occurring ICOS co-stimulatory domain protein. In embodiments, a variant or mutant of the ICOS co-stimulatory domain protein does not include deletions compared to the naturally occurring ICOS co-stimulatory domain protein. In embodiments, a variant or mutant of the ICOS co-stimulatory domain protein does not include insertions compared to the naturally occurring ICOS co-stimulatory domain protein. In embodiments, a variant or mutant of the ICOS co-stimulatory domain protein includes substitutions that are conservative substitutions compared to the naturally occurring ICOS co-stimulatory domain protein. In embodiments, the ICOS co-stimulatory domain includes all or a portion of the protein identified by the NCBI sequence reference NP_036224.1, or an isoform or naturally occurring mutant or variant thereof.

The term “OX-40 co-stimulatory domain” as provided herein includes any of the recombinant or naturally-occurring forms of the co-stimulatory domain of OX-40, or variants or homologs thereof that maintain OX-40 co-stimulatory domain activity (e.g., within at least 50%, 80%, 90%, 95%, 96%, 97%, 98%, 99% or 100% activity compared to the OX-40 co-stimulatory domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring OX-40 co-stimulatory domain polypeptide. In embodiments, the OX-40 co-stimulatory domain is a human OX-40 co-stimulatory domain protein. In embodiments, a variant or mutant of the OX-40 co-stimulatory domain protein includes no more than 5, 4, 3, 2, or 1 deletions compared to the naturally occurring OX-40 co-stimulatory domain protein. In embodiments, a variant or mutant of the OX-40 co-stimulatory domain protein includes no more than 5, 4, 3, 2, or 1 insertions compared to the naturally occurring OX-40 co-stimulatory domain protein. In embodiments, a variant or mutant of the OX-40 co-stimulatory domain protein does not include deletions compared to the naturally occurring OX-40 co-stimulatory domain protein. In embodiments, a variant or mutant of the OX-40 co-stimulatory domain protein does not include insertions compared to the naturally occurring OX-40 co-stimulatory domain protein. In embodiments, a variant or mutant of the OX-40 co-stimulatory domain protein includes substitutions that are conservative substitutions compared to the naturally occurring OX-40 co-stimulatory domain protein. In embodiments, the OX-40 co-stimulatory domain includes all or a portion of the protein identified by the NCBI sequence reference NP_003318.1, or an isoform or naturally occurring mutant or variant thereof.

The term “CTLA-4 co-stimulatory domain” as provided herein includes any of the recombinant or naturally-occurring forms of the co-stimulatory domain of CTLA-4, or variants or homologs thereof that maintain CTLA-4 co-stimulatory domain activity (e.g., within at least 50%, 80%, 90%, 95%, 96%, 97%, 98%, 99% or 100% activity compared to the CTLA-4 co-stimulatory domain). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CTLA-4 co-stimulatory domain polypeptide. In embodiments, CTLA-4 co-stimulatory domain protein is a human CTLA-4 co-stimulatory domain protein. In embodiments, the CTLA-4 co-stimulatory domain includes no more than 5, 4, 3, 2, or 1 deletions. In embodiments, the CTLA-4 co-stimulatory domain protein includes no more than 5, 4, 3, 2, or 1 insertions. In embodiments, the CTLA-4 co-stimulatory domain protein does not include deletions. In embodiments, CTLA-4 co-stimulatory domain protein does not include insertions. In embodiments, the CTLA-4 co-stimulatory domain protein includes substitutions that are conservative substitutions. In embodiments, the CTLA-4 co-stimulatory domain is a human CTLA-4 co-stimulatory domain protein. In embodiments, a variant or mutant of the CTLA-4 co-stimulatory domain protein includes no more than 5, 4, 3, 2, or 1 deletions compared to the naturally occurring CTLA-4 co-stimulatory domain protein. In embodiments, a variant or mutant of the CTLA-4 co-stimulatory domain protein includes no more than 5, 4, 3, 2, or 1 insertions compared to the naturally occurring CTLA-4 co-stimulatory domain protein. In embodiments, a variant or mutant of the CTLA-4 co-stimulatory domain protein does not include deletions compared to the naturally occurring CTLA-4 co-stimulatory domain protein. In embodiments, a variant or mutant of the CTLA-4 co-stimulatory domain protein does not include insertions compared to the naturally occurring CTLA-4 co-stimulatory domain protein. In embodiments, a variant or mutant of the CTLA-4 co-stimulatory domain protein includes substitutions that are conservative substitutions compared to the naturally occurring CTLA-4 co-stimulatory domain protein.

The term “CD3ζ intracellular T-cell signaling domain” as provided herein includes any of the recombinant or naturally-occurring forms of the CD3ζ intracellular T-cell signaling domain, or variants or homologs thereof that maintain CD3ζ intracellular T-cell signaling domain activity (e.g. within at least 50%, 80%, 90%, 95%, 96%, 97%, 98%, 99% or 100% activity compared to the CD3 intracellular T-cell signaling domain). In some aspects, the variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring CD3ζ intracellular T-cell signaling domain polypeptide. In embodiments, the CD3 intracellular T-cell signaling domain is a human CD3 intracellular T-cell signaling domain protein. In embodiments, a variant or mutant of the CD3ζ intracellular T-cell signaling domain protein includes no more than 5, 4, 3, 2, or 1 deletions compared to the naturally occurring CD3ζ intracellular T-cell signaling domain protein. In embodiments, a variant or mutant of the CD3ζ intracellular T-cell signaling domain protein includes no more than 5, 4, 3, 2, or 1 insertions compared to the naturally occurring CD3ζ intracellular T-cell signaling domain protein. In embodiments, a variant or mutant of the CD3ζ intracellular T-cell signaling domain protein does not include deletions compared to the naturally occurring CD3ζ intracellular T-cell signaling domain protein. In embodiments, a variant or mutant of the CD3ζ intracellular T-cell signaling domain protein does not include insertions compared to the naturally occurring CD3 intracellular T-cell signaling domain protein. In embodiments, a variant or mutant of the CD3ζ intracellular T-cell signaling domain protein includes substitutions that are conservative substitutions compared to the naturally occurring CD3 intracellular T-cell signaling domain protein. In embodiments, the CD3ζ intracellular T-cell signaling domain includes all or a portion of the protein identified by the NCBI sequence reference NP_000725.1, or an isoform or naturally occurring mutant or variant thereof. In embodiments, the CD3 intracellular T-cell signaling domain includes all or a portion of the protein identified by the NCBI sequence reference NP_932170.1, or an isoform or naturally occurring mutant or variant thereof. Non-limiting examples of human CD3-zeta amino acid sequences available under NCBI sequence references are identified supra.

In embodiments, the CD3 intracellular T-cell signaling domain is encoded by all or a portion of the nucleic acid sequence identified by the NCBI sequence reference NM_000734.3, or an isoform or naturally occurring mutant or variant thereof. In embodiments, the CD3ζ intracellular T-cell signaling domain is encoded by all or a portion of the nucleic acid sequence identified by the NCBI sequence reference NM_198053.2, or an isoform or naturally occurring mutant or variant thereof. In embodiments, a variant or mutant of the CD3ζ intracellular T-cell signaling domain nucleic acid sequence includes no more than 5, 4, 3, 2, or 1 deletions compared to the naturally occurring CD3ζ intracellular T-cell signaling domain nucleic acid sequence. In embodiments, a variant or mutant of the CD3ζ intracellular T-cell signaling domain nucleic acid sequence includes no more than 5, 4, 3, 2, or 1 insertions compared to the naturally occurring CD3ζ intracellular T-cell signaling domain nucleic acid sequence. In embodiments, a variant or mutant of the CD3ζ intracellular T-cell signaling domain nucleic acid sequence does not include deletions compared to the naturally occurring CD3ζ intracellular T-cell signaling domain nucleic acid sequence. In embodiments, a variant or mutant of the CD3ζ intracellular T-cell signaling domain nucleic acid sequence does not include insertions compared to the naturally occurring CD3ζ intracellular T-cell signaling domain nucleic acid sequence. In embodiments, a variant or mutant of the CD3ζ intracellular T-cell signaling domain nucleic acid sequence includes substitutions that are conservative substitutions compared to the naturally occurring CD3ζ intracellular T-cell signaling domain nucleic acid sequence.

“T cells” or “T lymphocytes” as used herein are a type of lymphocyte (a subtype of white blood cell) that plays a central role in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant.

As used herein, the term “cancer” or “tumor” refers to all types of cancer, neoplasm or malignant tumors found in mammals (e.g., humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

As used herein, the terms “neuroendocrine cancer” or “neuroendocrine tumor” refer to cancers or tumors that form from cells that release hormones into the blood in response to a signal from the nervous system. Neuroendocrine tumors may make higher-than-normal amounts of hormones, which can cause many different symptoms. Neuroendocrine tumors may be benign (not cancer) or malignant (cancer). Neuroendocrine cancers and tumors include, but are not limited to, carcinoid tumors, islet cell tumors, medullary thyroid cancer, pheochromocytomas, neuroendocrine carcinoma of the skin (Merkel cell cancer), small cell lung cancer, large cell neuroendocrine carcinoma, neuroendocrine prostate cancer (NEPC), and metastatic castration-resistant prostate cancer (CRPC).

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

“Treating“or treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms; fully or partially remove the disease's underlying cause; shorten a disease's duration; or do a combination of these things.

“Treating” and “treatment” as used herein also include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is no prophylactic treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

By “Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

An “anticancer agent” as used herein refers to a molecule (e.g. compound, peptide, protein, nucleic acid, 0103) used to treat cancer through destruction or inhibition of cancer cells or tissues. Anticancer agents may be selective for certain cancers or certain tissues. In embodiments, anticancer agents herein may include epigenetic inhibitors and multi-kinase inhibit “Anti-cancer agent” and “anticancer agent” are used in accordance with their plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39.HCl), AC-7700 (Ajinomoto, i.e. AVE-8062, AVE-8062A, CS-39-L-Ser.HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to 111In, 90Y, or 131I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™) erlotinib (Tarceva™), cetuximab (Erbitux™), lapatinib (Tykerb™), panitumumab (Vectibix™) vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

“Selective” or “selectivity” or the like of a compound refers to the compound's ability to discriminate between molecular targets (e.g. a compound having selectivity toward ROR1).

“Specific”, “specifically”, “specificity”, or the like of a compound refers to the compound's ability to cause a particular action, such as inhibition, to a particular molecular target with minimal or no action to other proteins in the cell.

An “ROR1 inhibitor” refers to a compound (e.g. compounds described herein) that reduces the activity of ROR1 when compared to a control, such as absence of the compound or a compound with known inactivity.

“Contacting” is used in accordance with its plain ordinary meaning and refers to the process of allowing at least two distinct species (e.g., chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

As defined herein, the term “inhibition”, “inhibit”, “inhibiting” and the like in reference to a protein-inhibitor interaction means negatively affecting (e.g., decreasing) the activity or function of the protein relative to the activity or function of the protein in the absence of the inhibitor. In embodiments inhibition means negatively affecting (e.g., decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g., an inhibitor binds to the target protein). In embodiments, inhibition refers to a reduction of activity of a target protein from an indirect interaction (e.g., an inhibitor binds to a protein that activates the target protein, thereby preventing target protein activation). A “ROR1 inhibitor” is a compound that negatively affects (e.g., decreases) the activity or function of ROR1 relative to the activity or function of ROR1 in the absence of the inhibitor.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “associated” or “associated with” in the context of a substance or substance activity or function associated with a disease (e.g. a protein associated disease, a cancer associated with ROR1 activity, ROR1 associated cancer, ROR1 associated disease (e.g., cancer, inflammatory disease, autoimmune disease, or infectious disease)) means that the disease (e.g., cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease. For example, a cancer associated with ROR1 activity or function or a ROR1 associated disease (e.g., cancer, inflammatory disease, autoimmune disease, or infectious disease), may be treated with a ROR1 modulator or ROR1 inhibitor, in the instance where increased ROR1 activity or function (e.g., signaling pathway activity) causes the disease (e.g., cancer, inflammatory disease, autoimmune disease, or infectious disease). For example, an inflammatory disease associated with ROR1 activity or function or an ROR1 associated inflammatory disease, may be treated with an ROR1 modulator or ROR1 inhibitor, in the instance where increased ROR1 activity or function (e.g. signaling pathway activity) causes the disease.

The term “signaling pathway” as used herein refers to a series of interactions between cellular and optionally extra-cellular components (e.g. proteins, nucleic acids, small molecules, ions, lipids) that conveys a change in one component to one or more other components, which in turn may convey a change to additional components, which is optionally propagated to other signaling pathway components.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Chimeric Antigen Receptors

Provided herein are recombinant protein/chimeric antigen receptor (these terms are used interchangeably throughout), nucleic acids encoding the recombinant proteins, cells expressing the chimeric antigen receptors disclosed herein, compositions thereof, and methods of using the same that are, inter alia, useful for treating a neuroendocrine cancer and for treating or preventing a metastasis originating from a neuroendocrine cancer. Applicants have discovered that chimeric antigen receptors (CARs) directed to ROR-1 provide for highly active and efficient immunotherapeutic compositions to treat a neuroendocrine cancer and treat or prevent metastasis in a subject with a neuroendocrine cancer. Without being bound to any particular theory, antibodies known to inhibit the receptor they bind and downregulate its surface expression are generally not considered good clinical candidates for CARs. Therefore, it was very surprising that CARs including CDRs of ROR-1 antibodies described herein including embodiments thereof exhibit effective neuroendocrine cancer-specific cytotoxicity when expressed by T cells.

In one aspect, the chimeric antigen receptor includes: an antibody binding region, wherein the antibody binding region specifically binds ROR-1, a transmembrane domain, a spacer domain that couples the antigen binding region and the transmembrane domain, and an intracellular domain. The terms “antibody region,” “antigen binding region,” or “antigen binding domain” as provided herein are used interchangeably throughout and refer to a monovalent or multivalent protein moiety that forms part of the protein (i.e. recombinant protein, chimeric antigen receptor) provided herein including embodiments thereof. A person of ordinary skill in the art would therefore immediately recognize that the antibody region or antigen binding region is a protein moiety capable of binding an antigen (epitope). The antibody region provided herein may include a domain of an antibody or fragment (e.g., Fab) thereof. Thus, the antibody region may include a light chain variable domain (VL) and/or a heavy chain variable domain (VH).

In embodiments, the ROR-1 binding domain includes an antibody region including a light chain variable region (VL) and a heavy chain variable region (VH). In embodiments, the light chain variable domain includes a CDR L1 as set forth in SEQ ID NO:43, a CDR L2 as set forth in SEQ ID NO:44 and a CDR L3 as set forth in SEQ ID NO:45, and the heavy chain variable domain including a CDR H1 as set forth in SEQ ID NO:46, a CDR H2 as set forth in SEQ ID NO:47, and a CDR H3 as set forth in SEQ ID NO:48. It is noted that those CDRs are of or are derived from cirmtuzumab (also known as UC-961 or 99961.1). The development and structure of cirmtuzumab is disclosed in U.S. Pat. No. 9,758,591, which is incorporated by reference herein in its entirety and for all purposes. In embodiments, the ROR-1 binding domain includes an antibody region including CDR L1 as set forth in SEQ ID NO:49, a CDR L2 as set forth in SEQ ID NO:50 and a CDR L3 as set forth in SEQ ID NO:51, and the heavy chain variable domain includes a CDR H1 as set forth in SEQ ID NO:52, a CDR H2 as set forth in SEQ ID NO:53, and a CDR H3 as set forth in SEQ ID NO:54.

In one aspect, the chimeric antigen receptor includes: i. an antibody binding region, wherein the antibody binding region specifically binds ROR-1, and wherein the antibody binding region includes a light chain variable domain and a heavy chain variable domain; (a) wherein the light chain variable domain includes a CDR L1 as set forth in SEQ ID NO:43, a CDR L2 as set forth in SEQ ID NO:44 and a CDR L3 as set forth in SEQ ID NO:45; and the heavy chain variable domain includes a CDR H1 as set forth in SEQ ID NO:46, a CDR H2 as set forth in SEQ ID NO:47, and a CDR H3 as set forth in SEQ ID NO:48; or (b) wherein the light chain variable domain includes a CDR L1 as set forth in SEQ ID NO:49, a CDR L2 as set forth in SEQ ID NO:50 and a CDR L3 as set forth in SEQ ID NO:51; and the heavy chain variable domain includes a CDR H1 as set forth in SEQ ID NO:52, a CDR H2 as set forth in SEQ ID NO:53, and a CDR H3 as set forth in SEQ ID NO:54; ii. a spacer domain, between 14 and 120 amino acids in length; iii. a transmembrane domain; and iv. an intracellular domain.

In one aspect, the chimeric antigen receptor includes: i. an antibody binding region, wherein the antibody binding region specifically binds ROR-1, and wherein the antibody binding region includes a light chain variable domain and a heavy chain variable domain; (a) wherein the light chain variable domain includes a CDR L1 as set forth in SEQ ID NO:43, a CDR L2 as set forth in SEQ ID NO:44 and a CDR L3 as set forth in SEQ ID NO:45; and the heavy chain variable domain includes a CDR H1 as set forth in SEQ ID NO:46, a CDR H2 as set forth in SEQ ID NO:47, and a CDR H3 as set forth in SEQ ID NO:48; or (b) wherein the light chain variable domain includes a CDR L1 as set forth in SEQ ID NO:49, a CDR L2 as set forth in SEQ ID NO:50 and a CDR L3 as set forth in SEQ ID NO:51; and the heavy chain variable domain includes a CDR H1 as set forth in SEQ ID NO:52, a CDR H2 as set forth in SEQ ID NO:53, and a CDR H3 as set forth in SEQ ID NO:54; ii. a spacer domain; iii. a transmembrane domain, wherein the transmembrane domain comprises a CD8α transmembrane domain, a CD28 transmembrane domain, a CD4 transmembrane domain, a CD3ζ transmembrane domain, or any combination thereof; and iv. an intracellular domain.

In one aspect, the chimeric antibody receptor includes: i. an antibody binding region, wherein the antibody binding region specifically binds ROR-1 and wherein the antibody binding region includes a light chain variable domain and a heavy chain variable domain; (a) wherein the light chain variable domain includes a CDR L1 as set forth in SEQ ID NO:43, a CDR L2 as set forth in SEQ ID NO:44 and a CDR L3 as set forth in SEQ ID NO:45; and the heavy chain variable domain includes a CDR H1 as set forth in SEQ ID NO:46, a CDR H2 as set forth in SEQ ID NO:47, and a CDR H3 as set forth in SEQ ID NO:48; or (b) wherein said light chain variable domain includes a CDR L1 as set forth in SEQ ID NO:49, a CDR L2 as set forth in SEQ ID NO:50 and a CDR L3 as set forth in SEQ ID NO:51; and the heavy chain variable domain including a CDR H1 as set forth in SEQ ID NO:52, a CDR H2 as set forth in SEQ ID NO:53, and a CDR H3 as set forth in SEQ ID NO:54; ii. a linker domain; iii. a transmembrane domain; and iv. an intracellular domain, wherein the intracellular domain includes an intracellular T cell signaling domain and an intracellular co-stimulatory domain selected from, 4-1BB, ICOS, OX-40, and combinations thereof.

A light chain variable (VL) domain as provided includes CDR sequences and framework region (FR) sequences of the light chain of an antibody, an antibody variant or fragment thereof. In embodiments, the antibody region or antigen binding region includes a variable light chain domain and a variable heavy chain domain. A “variable light chain domain” as provided herein refers to a polypeptide included in (forming part of) a light chain variable (VL) region. In embodiments, the variable light chain region is a light chain variable (VL) domain. A “variable heavy chain domain” as provided herein refers to a polypeptide included in (forming part of) a heavy chain variable (VH) region. In embodiments, the variable heavy chain region is a heavy chain variable (VH) domain. In embodiments, the light chain variable (VL) domain includes CDR L1 (SEQ ID NO:43), CDR L2 (SEQ ID NO:44), and CDR L3 (SEQ ID NO:45). In embodiments, the heavy chain variable (VH) domain includes CDR H1 (SEQ ID NO:46), CDR H2 (SEQ ID NO:47), and CDR H3 (SEQ ID NO:48).

In embodiments, the light chain variable domain includes the amino acid sequence of SEQ ID NO:21, or an amino acid sequence at least 80%, 85%, 90%, or 95% identical to SEQ ID NO:21. In embodiments, the light chain variable domain has the amino acid sequence of SEQ ID NO:21, or an amino acid sequence at least 80%, 85%, 90%, or 95% identical to SEQ ID NO:21. In embodiments, the heavy chain variable domain includes the amino acid sequence of SEQ ID NO:27, or an amino acid sequence at least 80%, 85%, 90%, or 95% identical to SEQ ID NO:27. In embodiments, the heavy chain variable domain has the amino acid sequence of SEQ ID NO:27, or an amino acid sequence at least 80%, 85%, 90%, or 95% identical to SEQ ID NO:27.

In embodiments, the light chain variable domain includes the amino acid sequence of SEQ ID NO:19, or an amino acid sequence at least 80%, 85%, 90%, or 95% identical to SEQ ID NO:19. In embodiments, the light chain variable domain has the amino acid sequence of SEQ ID NO: 19, or an amino acid sequence at least 80%, 85%, 90%, or 95% identical to SEQ ID NO:19. In embodiments, the light chain variable domain includes the amino acid sequence of SEQ ID NO:20, or a sequence at least 80%, 85%, 90%, or 95% identical to SEQ ID NO:20. In embodiments, the light chain variable domain has the amino acid sequence of SEQ ID NO:20, or an amino acid sequence at least 80%, 85%, 90%, or 95% identical to SEQ ID NO:20. In embodiments, the heavy chain variable domain includes the amino acid sequence of SEQ ID NO:25, or an amino acid sequence at least 80%, 85%, 90%, or 95% identical to SEQ ID NO:25. In embodiments, the heavy chain variable domain has the amino acid sequence of SEQ ID NO:25, or an amino acid sequence at least 80%, 85%, 90%, or 95% identical to SEQ ID NO:25. In embodiments, the heavy chain variable domain includes the amino acid sequence of SEQ ID NO:26, or an amino acid sequence at least 80%, 85%, 90%, or 95% identical to SEQ ID NO:26. In embodiments, the heavy chain variable domain has the amino acid sequence of SEQ ID NO:26, or an amino acid sequence at least 80%, 85%, 90%, or 95% identical to SEQ ID NO:26.

In embodiments, the C-terminus of the light chain variable domain is bound to the N-terminus of the heavy chain variable domain. In embodiments, the N-terminus of the light chain variable domain is bound to the C-terminus of the heavy chain variable domain. In embodiments, the light chain variable domain is covalently bound to the heavy chain variable domain through a chemical linker. A “chemical linker,” as provided herein, is a covalent linker, a non-covalent linker, a peptide linker (a linker including a peptide moiety), a cleavable peptide linker, a substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene or any combination thereof. Thus, a chemical linker as provided herein may include a plurality of chemical moieties, wherein each of the plurality of chemical moieties is chemically different. Alternatively, the chemical linker may be a non-covalent linker. Examples of non-covalent linkers include without limitation, ionic bonds, hydrogen bonds, halogen bonds, van der Waals interactions (e.g. dipole-dipole, dipole-induced dipole, London dispersion), ring stacking (pi effects), and hydrophobic interactions. In embodiments, a chemical linker is formed using conjugate chemistry including, but not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). In embodiments, the chemical linker is a peptide linker, which comprises 5-100, 5-80, 5-70, 5-60, 5-50, 10-50, 10-40, or 10-30 amino acids in length. Any sequence that provides sufficient flexibility between the light chain variable domain and the heavy chain variable domain is contemplated for the recombinant proteins provided herein including embodiments thereof. In embodiments, the peptide linker includes or has the amino acid sequence of SEQ ID NO:24. In embodiments, the peptide linker has an amino acid sequence at least 80%, 85%, 90%, or 95% identical to SEQ ID NO:24.

The term “transmembrane domain” refers to a membrane-spanning protein domain capable of anchoring a protein to the membrane. Any transmembrane domain capable of anchoring the proteins provided herein including embodiments thereof are contemplated. While any suitable transmembrane domains are contemplated herein, exemplary, but non-limiting examples of transmembrane domains include the transmembrane domains of CD28, CD8, CD4, CD3ζ, or CD8α. In some embodiments, the transmembrane domain is a CD28 transmembrane domain. The term “CD28 transmembrane domain” as provided herein includes any of the recombinant or naturally-occurring forms of the transmembrane domain of CD28, or variants or homologs thereof that maintain CD28 transmembrane domain activity (e.g., within at least 50%, 80%, 90%, 95%, 96%, 97%, 98%, 99% or 100% activity compared to the CD28 transmembrane domain). In some aspects, the variants or homologs have at least 80%, 85%, 90%, 95%, 96%, 97%, 98%, or 99% amino acid sequence identity across the whole sequence or a portion of the a naturally occurring CD28 transmembrane domain polypeptide. In embodiments, the CD28 transmembrane domain includes an amino acid sequence of SEQ ID NO:32. In embodiments, the CD28 transmembrane domain is an amino acid sequence of SEQ ID NO:32. In embodiments, the CD28 transmembrane domain in the ROR-1 CAR described herein has a sequence at least 80%, 85%, 90%, or 95% identical to SEQ ID NO:32.

In embodiments, the C-terminus of the heavy chain variable domain is coupled to the N-terminus of the transmembrane domain. In embodiments, the C-terminus of the light chain variable domain is coupled to the N-terminus of the transmembrane domain. In embodiments, the heavy chain variable domain or light chain variable domain is covalently bound to the transmembrane domain through a spacer domain. In embodiments, the binding affinity of the antigen binding region to the antigen is increased in the CAR construct having the spacer domain. In embodiments, the activity of the CAR having the spacer domain is higher than the CAR without the spacer domain. In embodiments, the flexibility of the antigen binding region of the CAR is increased by the presence of the spacer domain.

The term “intracellular domain” refers to the portion of the receptor or protein that interacts with the interior of a cell or cell organelle via protein-protein interactions. In embodiments, the recombinant protein (e.g., the intracellular domain) further includes one or more intracellular co-stimulatory signaling domains. An “intracellular co-stimulatory signaling domain” as provided herein includes amino acid sequences capable of providing co-stimulatory signaling in response to binding of an antigen to the antibody region provided herein including embodiments thereof. In embodiments, the signaling of the co-stimulatory signaling domain results in production of cytokines and proliferation of the T cell expressing the same. In embodiments, the intracellular co-stimulatory signaling domain comprises at least one or more of a CD28 intracellular co-stimulatory signaling domain, a 4-1BB (CD137) intracellular co-stimulatory signaling domain, an ICOS intracellular co-stimulatory signaling domain, an OX-40 intracellular co-stimulatory signaling domain, or any combinations thereof. For example, a preferred intracellular co-stimulatory signaling domain comprises or consists of at least a portion of CD28 intracellular co-stimulatory signaling domain, or at least a portion of a 4-1BB (CD137) intracellular co-stimulatory signaling domain. Another preferred intracellular co-stimulatory signaling domain comprises or consists of at least a portion of CD28 intracellular co-stimulatory signaling domain coupled with at least a portion of a 4-1BB (CD137) intracellular co-stimulatory signaling domain. In embodiments, at least a portion of the CD28 intracellular domain comprises or consists of the amino acid sequence of SEQ ID NO:32 or an amino acid sequence at least 80%, 85%, 90%, or 95% identical to SEQ ID NO:32. In embodiments, at least a portion of the CD28 intracellular domain comprises or consists of the amino acid sequence of SEQ ID NO:32 or an amino acid sequence at least 80%, 85%, 90%, or 95% identical to SEQ ID NO:32. In embodiments, at least a portion of the 4-1BB intracellular co-stimulatory signaling domain comprises or consists of the amino acid sequence of SEQ ID NO:33 or an amino acid sequence at least 80%, 85%, 90%, or 95% identical to SEQ ID NO:33. In embodiments, at least a portion of the 4-1BB intracellular co-stimulatory signaling domain comprises or consists of the amino acid sequence of SEQ ID NO:33 or an amino acid sequence at least 80%, 85%, 90%, or 95% identical to SEQ ID NO:29. In embodiments, the chimeric antigen receptor disclosed herein or the recombinant protein includes an amino acid sequence of a combination of SEQ ID NO: 32 and SEQ ID NO:33 (either SEQ ID NO: 32 present at the N′-terminus of SEQ ID NO: 33, or SEQ ID NO: 33 present at the N′-terminus of SEQ ID NO: 32), or an amino acid sequence at least 80%, 85%, 90%, or 95% identical to a combination of SEQ ID NO: 32 and SEQ ID NO:33 (either SEQ ID NO: 32 present at the N′-terminus of SEQ ID NO: 33, or SEQ ID NO: 33 present at the N′-terminus of SEQ ID NO: 32).

In embodiments, the recombinant protein further includes an intracellular T-cell signaling domain. An “intracellular T-cell signaling domain” as provided herein includes amino acid sequences capable of providing primary signaling in response to binding of an antigen to the antibody region provided herein including embodiments thereof. In embodiments, the signaling of the intracellular T-cell signaling domain results in activation of the T cell expressing the chimeric antigen receptor. In embodiments, the signaling of the intracellular T-cell signaling domain results in proliferation (cell division) of the T cell expressing the same. In embodiments, the signaling of the intracellular T-cell signaling domain results in the expression of proteins by the T cell that are known in the art to be characteristic of activated T cell such as, for example, CTLA-4, PD-1, CD28, C and D69. In embodiments, the intracellular T-cell signaling domain is a CD3ζ intracellular T-cell signaling domain.

In embodiments, the CD3ζ intracellular T-cell signaling domain includes the amino acid sequence of SEQ ID NO:34 or an amino acid sequence at least 80%, 85%, 90%, or 95% identical to SEQ ID NO:34 that generates a CD3ζ intracellular T-cell signaling domain having at least 80%, 85%, 90%, or 95% activity of the CD3ζ intracellular T-cell signaling domain having SEQ ID NO:34. In embodiments, the CD3ζ intracellular T-cell signaling domain has the amino acid sequence of SEQ ID NO:34 or a sequence at least 80%, 85%, 90%, or 95% identical to SEQ ID NO:34 that generates a peptide having at least 80%, 85%, 90%, or 95% activity of the CD3ζ intracellular T-cell signaling domain having SEQ ID NO:34.

In embodiments, the ROR-1 CAR disclosed herein includes or consists of i) ROR1 scFv (having CDR L1 (SEQ ID NO:43), CDR L2 (SEQ ID NO:44), and CDR L3 (SEQ ID NO:45), CDR H1 (SEQ ID NO:46), CDR H2 (SEQ ID NO:47), and CDR H3 (SEQ ID NO:48), or VL domain having the sequence of SEQ ID NO:21, or a sequence at least 80%, 85%, 90%, or 95% identical to SEQ ID NO:21 and the VH domain having the sequence of SEQ ID NO:27, or a sequence at least 80%, 85%, 90%, or 95% identical to SEQ ID NO:27) or alternatively ROR1 scFv (CDR L1 (SEQ ID NO:49), CDR L2 (SEQ ID NO:50), and CDR L3 (SEQ ID NO:51), CDR H1 (SEQ ID NO:52), CDR H2 (SEQ ID NO:53), and CDR H3 (SEQ ID NO:54), or VL domain having the sequence of SEQ ID NO:19, or a sequence at least 80%, 85%, 90%, or 95% identical to SEQ ID NO:19 and the VH domain having the sequence of SEQ ID NO:20, or a sequence at least 80%, 85%, 90%, or 95% identical to SEQ ID NO:20), ii) a spacer domain having a hinge and CH2 and CH3 domain, iii) CD28 transmembrane domain, iv) 4-1BB (CD137) costimulatory domain, and v) CD3Z T cell activation domain.

Alternatively, in embodiments, the ROR1 CAR disclosed herein includes or consists of i) ROR1 scFv (having CDR L1 (SEQ ID NO:43), CDR L2 (SEQ ID NO:44), and CDR L3 (SEQ ID NO:45), CDR H1 (SEQ ID NO:46), CDR H2 (SEQ ID NO:47), and CDR H3 (SEQ ID NO:48), or VL domain having the sequence of SEQ ID NO:21, or a sequence at least 80%, 85%, 90%, or 95% identical to SEQ ID NO:21 and the VH domain having the sequence of SEQ ID NO:27, or a sequence at least 80%, 85%, 90%, or 95% identical to SEQ ID NO:27) or alternatively ROR1 scFv (CDR L1 (SEQ ID NO:49), CDR L2 (SEQ ID NO:50), and CDR L3 (SEQ ID NO:51), CDR H1 (SEQ ID NO:52), CDR H2 (SEQ ID NO:53), and CDR H3 (SEQ ID NO:54), or VL domain having the sequence of SEQ ID NO:19, or a sequence at least 80%, 85%, 90%, or 95% identical to SEQ ID NO:19 and the VH domain having the sequence of SEQ ID NO:20, or a sequence at least 80%, 85%, 90%, or 95% identical to SEQ ID NO:20), ii) a spacer domain having a hinge and CH2 domain, iii) CD28 transmembrane domain, iv) 4-1BB (CD137) costimulatory domain, and v) CD3Z T cell activation domain.

Alternatively, in embodiments, the ROR1 CAR disclosed herein includes or consists of i) ROR1 scFv (having CDR L1 (SEQ ID NO:43), CDR L2 (SEQ ID NO:44), and CDR L3 (SEQ ID NO:45), CDR H1 (SEQ ID NO:46), CDR H2 (SEQ ID NO:47), and CDR H3 (SEQ ID NO:48), or VL domain having the sequence of SEQ ID NO:21, or a sequence at least 80%, 85%, 90%, or 95% identical to SEQ ID NO:21 and the VH domain having the sequence of SEQ ID NO:27, or a sequence at least 80%, 85%, 90%, or 95% identical to SEQ ID NO:27) or alternatively ROR1 scFv (CDR L1 (SEQ ID NO:49), CDR L2 (SEQ ID NO:50), and CDR L3 (SEQ ID NO:51), CDR H1 (SEQ ID NO:52), CDR H2 (SEQ ID NO:53), and CDR H3 (SEQ ID NO:54), or VL domain having the sequence of SEQ ID NO:19, or a sequence at least 80%, 85%, 90%, or 95% identical to SEQ ID NO:19 and the VH domain having the sequence of SEQ ID NO:20, or a sequence at least 80%, 85%, 90%, or 95% identical to SEQ ID NO:20), ii) a spacer domain having a hinge and CH3 domain, iii) CD28 transmembrane domain, iv) 4-1BB (CD137) costimulatory domain, and v) CD3Z T cell activation domain.

Alternatively, in embodiments, the ROR1 CAR disclosed herein includes or consists of i) ROR1 scFv (having CDR L1 (SEQ ID NO:43), CDR L2 (SEQ ID NO:44), and CDR L3 (SEQ ID NO:45), CDR H1 (SEQ ID NO:46), CDR H2 (SEQ ID NO:47), and CDR H3 (SEQ ID NO:48), or VL domain having the sequence of SEQ ID NO:21, or a sequence at least 80%, 85%, 90%, or 95% identical to SEQ ID NO:21 and the VH domain having the sequence of SEQ ID NO:27, or a sequence at least 80%, 85%, 90%, or 95% identical to SEQ ID NO:27) or alternatively ROR1 scFv (CDR L1 (SEQ ID NO:49), CDR L2 (SEQ ID NO:50), and CDR L3 (SEQ ID NO:51), CDR H1 (SEQ ID NO:52), CDR H2 (SEQ ID NO:53), and CDR H3 (SEQ ID NO:54), or VL domain having the sequence of SEQ ID NO:19, or a sequence at least 80%, 85%, 90%, or 95% identical to SEQ ID NO:19 and the VH domain having the sequence of SEQ ID NO:20, or a sequence at least 80%, 85%, 90%, or 95% identical to SEQ ID NO:20), ii) a spacer domain having a hinge and a portion of CH3 domain (e.g., a half of CH3 domain, N-terminal 43 amino acids of CH3 domain, etc.), iii) CD28 transmembrane domain, iv) 4-1BB (CD137) costimulatory domain, and v) CD3Z T cell activation domain.

Alternatively, in embodiments, the ROR1 CAR disclosed herein includes or consists of i) ROR1 scFv (having CDR L1 (SEQ ID NO:43), CDR L2 (SEQ ID NO:44), and CDR L3 (SEQ ID NO:45), CDR H1 (SEQ ID NO:46), CDR H2 (SEQ ID NO:47), and CDR H3 (SEQ ID NO:48), or VL domain having the sequence of SEQ ID NO:21, or a sequence at least 80%, 85%, 90%, or 95% identical to SEQ ID NO:21 and the VH domain having the sequence of SEQ ID NO:27, or a sequence at least 80%, 85%, 90%, or 95% identical to SEQ ID NO:27) or alternatively ROR1 scFv (CDR L1 (SEQ ID NO:49), CDR L2 (SEQ ID NO:50), and CDR L3 (SEQ ID NO:51), CDR H1 (SEQ ID NO:52), CDR H2 (SEQ ID NO:53), and CDR H3 (SEQ ID NO:54), or VL domain having the sequence of SEQ ID NO:19, or a sequence at least 80%, 85%, 90%, or 95% identical to SEQ ID NO:19 and the VH domain having the sequence of SEQ ID NO:20, or a sequence at least 80%, 85%, 90%, or 95% identical to SEQ ID NO:20), ii) a spacer domain having a hinge domain, iii) CD28 transmembrane domain, iv) 4-1BB (CD137) costimulatory domain, and v) CD3Z T cell activation domain.

Alternatively, in embodiments, the ROR1 CAR disclosed herein includes or consists of i) ROR1 scFv (having CDR L1 (SEQ ID NO:43), CDR L2 (SEQ ID NO:44), and CDR L3 (SEQ ID NO:45), CDR H1 (SEQ ID NO:46), CDR H2 (SEQ ID NO:47), and CDR H3 (SEQ ID NO:48), or VL domain having the sequence of SEQ ID NO:21, or a sequence at least 80%, 85%, 90%, or 95% identical to SEQ ID NO:21 and the VH domain having the sequence of SEQ ID NO:27, or a sequence at least 80%, 85%, 90%, or 95% identical to SEQ ID NO:27) or alternatively ROR1 scFv (CDR L1 (SEQ ID NO:49), CDR L2 (SEQ ID NO:50), and CDR L3 (SEQ ID NO:51), CDR H1 (SEQ ID NO:52), CDR H2 (SEQ ID NO:53), and CDR H3 (SEQ ID NO:54), or VL domain having the sequence of SEQ ID NO:19, or a sequence at least 80%, 85%, 90%, or 95% identical to SEQ ID NO:19 and the VH domain having the sequence of SEQ ID NO:20, or a sequence at least 80%, 85%, 90%, or 95% identical to SEQ ID NO:20), ii) a spacer domain having a hinge and CH2 and CH3 domain, iii) CD28 transmembrane domain, iv) CD28 costimulatory domain coupled to 4-1BB (CD137) costimulatory domain, and v) CD3Z T cell activation domain.

Alternatively, in embodiments, the ROR-1 CAR disclosed herein includes or consists of i) ROR1 scFv (having CDR L1 (SEQ ID NO:43), CDR L2 (SEQ ID NO:44), and CDR L3 (SEQ ID NO:45), CDR H1 (SEQ ID NO:46), CDR H2 (SEQ ID NO:47), and CDR H3 (SEQ ID NO:48), or VL domain having the sequence of SEQ ID NO:21, or a sequence at least 80%, 85%, 90%, or 95% identical to SEQ ID NO:21 and the VH domain having the sequence of SEQ ID NO:27, or a sequence at least 80%, 85%, 90%, or 95% identical to SEQ ID NO:27) or alternatively ROR1 scFv (CDR L1 (SEQ ID NO:49), CDR L2 (SEQ ID NO:50), and CDR L3 (SEQ ID NO:51), CDR H1 (SEQ ID NO:52), CDR H2 (SEQ ID NO:53), and CDR H3 (SEQ ID NO:54), or VL domain having the sequence of SEQ ID NO:19, or a sequence at least 80%, 85%, 90%, or 95% identical to SEQ ID NO:19 and the VH domain having the sequence of SEQ ID NO:20, or a sequence at least 80%, 85%, 90%, or 95% identical to SEQ ID NO:20), ii) a spacer domain having a hinge and CH2 domain, iii) CD28 transmembrane domain, iv) CD28 costimulatory domain coupled to 4-1BB (CD137) costimulatory domain, and v) CD3Z T cell activation domain.

Alternatively, in embodiments, the ROR-1 CAR disclosed herein includes or consists of i) ROR1 scFv (having CDR L1 (SEQ ID NO:43), CDR L2 (SEQ ID NO:44), and CDR L3 (SEQ ID NO:45), CDR H1 (SEQ ID NO:46), CDR H2 (SEQ ID NO:47), and CDR H3 (SEQ ID NO:48), or VL domain having the sequence of SEQ ID NO:21, or a sequence at least 80%, 85%, 90%, or 95% identical to SEQ ID NO:21 and the VH domain having the sequence of SEQ ID NO:27, or a sequence at least 80%, 85%, 90%, or 95% identical to SEQ ID NO:27) or alternatively ROR1 scFv (CDR L1 (SEQ ID NO:49), CDR L2 (SEQ ID NO:50), and CDR L3 (SEQ ID NO:51), CDR H1 (SEQ ID NO:52), CDR H2 (SEQ ID NO:53), and CDR H3 (SEQ ID NO:54), or VL domain having the sequence of SEQ ID NO:19, or a sequence at least 80%, 85%, 90%, or 95% identical to SEQ ID NO:19 and the VH domain having the sequence of SEQ ID NO:20, or a sequence at least 80%, 85%, 90%, or 95% identical to SEQ ID NO:20), ii) a spacer domain having a hinge and CH3 domain, iii) CD28 transmembrane domain, iv) CD28 costimulatory domain coupled to 4-1BB (CD137) costimulatory domain, and v) CD3Z T cell activation domain.

Alternatively, in embodiments, the ROR-1 CAR disclosed herein includes or consists of i) ROR1 scFv (having CDR L1 (SEQ ID NO:43), CDR L2 (SEQ ID NO:44), and CDR L3 (SEQ ID NO:45), CDR H1 (SEQ ID NO:46), CDR H2 (SEQ ID NO:47), and CDR H3 (SEQ ID NO:48), or VL domain having the sequence of SEQ ID NO:21, or a sequence at least 80%, 85%, 90%, or 95% identical to SEQ ID NO:21 and the VH domain having the sequence of SEQ ID NO:27, or a sequence at least 80%, 85%, 90%, or 95% identical to SEQ ID NO:27) or alternatively ROR1 scFv (CDR L1 (SEQ ID NO:49), CDR L2 (SEQ ID NO:50), and CDR L3 (SEQ ID NO:51), CDR H1 (SEQ ID NO:52), CDR H2 (SEQ ID NO:53), and CDR H3 (SEQ ID NO:54), or VL domain having the sequence of SEQ ID NO:19, or a sequence at least 80%, 85%, 90%, or 95% identical to SEQ ID NO:19 and the VH domain having the sequence of SEQ ID NO:20, or a sequence at least 80%, 85%, 90%, or 95% identical to SEQ ID NO:20), ii) a spacer domain having a hinge and a portion of CH3 domain (e.g., a half of CH3 domain, N-terminal 43 amino acids of CH3 domain, etc.), iii) CD28 transmembrane domain, iv) CD28 costimulatory domain coupled to 4-1BB (CD137) costimulatory domain, and v) CD3Z T cell activation domain.

Alternatively, in embodiments, the ROR-1 CAR disclosed herein includes or consists of i) ROR1 scFv (having CDR L1 (SEQ ID NO:43), CDR L2 (SEQ ID NO:44), and CDR L3 (SEQ ID NO:45), CDR H1 (SEQ ID NO:46), CDR H2 (SEQ ID NO:47), and CDR H3 (SEQ ID NO:48), or VL domain having the sequence of SEQ ID NO:21, or a sequence at least 80%, 85%, 90%, or 95% identical to SEQ ID NO:21 and the VH domain having the sequence of SEQ ID NO:27, or a sequence at least 80%, 85%, 90%, or 95% identical to SEQ ID NO:27) or alternatively ROR1 scFv (CDR L1 (SEQ ID NO:49), CDR L2 (SEQ ID NO:50), and CDR L3 (SEQ ID NO:51), CDR H1 (SEQ ID NO:52), CDR H2 (SEQ ID NO:53), and CDR H3 (SEQ ID NO:54), or VL domain having the sequence of SEQ ID NO:19, or a sequence at least 80%, 85%, 90%, or 95% identical to SEQ ID NO:19 and the VH domain having the sequence of SEQ ID NO:20, or a sequence at least 80%, 85%, 90%, or 95% identical to SEQ ID NO:20), ii) a spacer domain having a hinge domain, iii) CD28 transmembrane domain, iv) CD28 costimulatory domain coupled to 4-1BB (CD137) costimulatory domain, and v) CD3Z T cell activation domain.

It is contemplated that the chimeric antigen receptor described here binds to amino acids 130-160 of ROR-1 or a fragment thereof, preferably to a peptide including a glutamic acid at a position corresponding to position 138 of ROR-1 polypeptide. Alternatively and/or additionally, the chimeric antigen receptor described herein specifically binds either the 3′ or middle Ig-like region of the extracellular domain of the ROR-1 protein, preferably to 3′ end of the Ig-like region of the extracellular domain of ROR-1 protein from position 1-147.

Consequently, the ROR-1 CAR disclosed herein binds to a ROR-1 expressing cells and can initiate or induce immune response against the ROR-1 expressing cells, including a neuroendocrine cancer cell, such as, for example, a carcinoid tumor, an islet cell tumor, a medullary thyroid cancer, a pheochromocytoma, a neuroendocrine carcinoma of the skin, small cell lung cancer, large cell neuroendocrine carcinoma, neuroendocrine prostate cancer (NEPC), or metastatic castration-resistant prostate cancer (CRPC).

In embodiments, the recombinant protein provided herein, including embodiments thereof, further includes a detectable domain. A “detectable domain” as provided herein is a peptide moiety detectable by spectroscopic, photochemical, biochemical, immunochemical, chemical, or other physical means. For example, a detectable domain as provided herein may be a protein or other entity which can be made detectable, e.g., by incorporating a radiolabel or being reactive to an antibody specifically. Any appropriate method known in the art for conjugating an antibody to the label may be employed, e.g., using methods described in Hermanson, Bioconjugate Techniques 1996, Academic Press, Inc., San Diego. In the present invention, a detectable domain is used to confirm transfection of T cells.

In embodiments, the recombinant protein or chimeric antigen receptor has a binding affinity of about 500 pM to about 6 nM. In embodiments, the recombinant protein or chimeric antigen receptor has a binding affinity of about 550 pM to about 6 nM. In embodiments, the recombinant protein or chimeric antigen receptor has a binding affinity of about 600 pM to about 6 nM. In embodiments, the recombinant protein or chimeric antigen receptor has a binding affinity of about 650 pM to about 6 nM. In embodiments, the recombinant protein or chimeric antigen receptor has a binding affinity of about 700 pM to about 6 nM. In embodiments, the recombinant protein or chimeric antigen receptor has a binding affinity of about 750 pM to about 6 nM. In embodiments, the recombinant protein or chimeric antigen receptor has a binding affinity of about 800 pM to about 6 nM. In embodiments, the recombinant protein or chimeric antigen receptor has a binding affinity of about 850 pM to about 6 nM. In embodiments, the recombinant protein or chimeric antigen receptor has a binding affinity of about 900 pM to about 6 nM. In embodiments, the recombinant protein or chimeric antigen receptor has a binding affinity of about 950 pM to about 6 nM. In embodiments, the recombinant protein or chimeric antigen receptor has a binding affinity of about 1 nM to about 6 nM. In embodiments, the recombinant protein or chimeric antigen receptor has a binding affinity of about 1 nM to about 6 nM. In embodiments, the recombinant protein or chimeric antigen receptor has a binding affinity of about 1.5 nM to about 6 nM. In embodiments, the recombinant protein or chimeric antigen receptor has a binding affinity of about 2 nM to about 6 nM. In embodiments, the recombinant protein or chimeric antigen receptor has a binding affinity of about 2.5 nM to about 6 nM. In embodiments, the recombinant protein or chimeric antigen receptor has a binding affinity of about 3 nM to about 6 nM. In embodiments, the recombinant protein or chimeric antigen receptor has a binding affinity of about 3.5 nM to about 6 nM. In embodiments, the recombinant protein or chimeric antigen receptor has a binding affinity of about 4 nM to about 6 nM. In embodiments, the recombinant protein or chimeric antigen receptor has a binding affinity of about 4.5 nM to about 6 nM. In embodiments, the recombinant protein or chimeric antigen receptor has a binding affinity of about 5 nM to about 6 nM. In embodiments, the recombinant protein or chimeric antigen receptor has a binding affinity of about 5.5 nM to about 6 nM.

In embodiments, the recombinant protein or chimeric antigen receptor has a binding affinity of about 500 pM. In embodiments, the recombinant protein or chimeric antigen receptor has a binding affinity of 500 pM. In embodiments, the recombinant protein or chimeric antigen receptor has a binding affinity of about 550 pM. In embodiments, the recombinant protein or chimeric antigen receptor has a binding affinity of 550 pM. In embodiments, the recombinant protein or chimeric antigen receptor has a binding affinity of about 600 pM. In embodiments, the recombinant protein or chimeric antigen receptor has a binding affinity of 600 pM. In embodiments, the recombinant protein or chimeric antigen receptor has a binding affinity of about 650 pM. In embodiments, the recombinant protein or chimeric antigen receptor has a binding affinity of 650 pM. In embodiments, the recombinant protein or chimeric antigen receptor has a binding affinity of about 700 pM. In embodiments, the recombinant protein or chimeric antigen receptor has a binding affinity of 700 pM. In embodiments, the recombinant protein or chimeric antigen receptor has a binding affinity of about 750 pM. In embodiments, the recombinant protein or chimeric antigen receptor has a binding affinity of 750 pM. In embodiments, the recombinant protein or chimeric antigen receptor has a binding affinity of about 800 pM. In embodiments, the recombinant protein or chimeric antigen receptor has a binding affinity of 800 pM. In embodiments, the recombinant protein or chimeric antigen receptor has a binding affinity of about 850 pM. In embodiments, the recombinant protein or chimeric antigen receptor has a binding affinity of 850 pM. In embodiments, the recombinant protein or chimeric antigen receptor has a binding affinity of about 900 pM. In embodiments, the recombinant protein or chimeric antigen receptor has a binding affinity of 900 pM. In embodiments, the recombinant protein or chimeric antigen receptor has a binding affinity of about 950 pM. In embodiments, the recombinant protein or chimeric antigen receptor has a binding affinity of 950 pM. In embodiments, the recombinant protein or chimeric antigen receptor has a binding affinity of about 1 nM. In embodiments, the recombinant protein or chimeric antigen receptor has a binding affinity of about 1 nM. In embodiments, the recombinant protein or chimeric antigen receptor has a binding affinity of 1 nM. In embodiments, the recombinant protein or chimeric antigen receptor has a binding affinity of about 1.5 nM. In embodiments, the recombinant protein or chimeric antigen receptor has a binding affinity of 1.5 nM. In embodiments, the recombinant protein or chimeric antigen receptor has a binding affinity of about 2 nM. In embodiments, the recombinant protein or chimeric antigen receptor has a binding affinity of 2 nM. In embodiments, the recombinant protein or chimeric antigen receptor has a binding affinity of about 2.5 nM. In embodiments, the recombinant protein or chimeric antigen receptor has a binding affinity of 2.5 nM. In embodiments, the recombinant protein or chimeric antigen receptor has a binding affinity of about 3 nM. In embodiments, the recombinant protein or chimeric antigen receptor has a binding affinity of 3 nM. In embodiments, the recombinant protein or chimeric antigen receptor has a binding affinity of about 3.5 nM. In embodiments, the recombinant protein or chimeric antigen receptor has a binding affinity of 3.5 nM. In embodiments, the recombinant protein or chimeric antigen receptor has a binding affinity of about 4 nM. In embodiments, the recombinant protein or chimeric antigen receptor has a binding affinity of 4 nM. In embodiments, the recombinant protein or chimeric antigen receptor has a binding affinity of about 4.5 nM. In embodiments, the recombinant protein or chimeric antigen receptor has a binding affinity of 4.5 nM. In embodiments, the recombinant protein or chimeric antigen receptor has a binding affinity of about 5 nM. In embodiments, the recombinant protein or chimeric antigen receptor has a binding affinity of 5 nM. In embodiments, the recombinant protein or chimeric antigen receptor has a binding affinity of about 5.5 nM. In embodiments, the recombinant protein or chimeric antigen receptor has a binding affinity of 5.5 nM. In embodiments, the recombinant protein or chimeric antigen receptor has a binding affinity of about 6 nM. In embodiments, the recombinant protein or chimeric antigen receptor has a binding affinity of 6 nM.

In embodiments, the recombinant protein or chimeric antigen receptor binds to an ROR-1 protein with a KD of less than about 40 nM (e.g., 35, 30, 25, 20, 15, 10, 9, 8, 7, 6, 5, 4, 3, 2, 1, 0.5, 0.25, 0.1 nM). In embodiments, the recombinant protein or chimeric antigen receptor binds to an ROR-1 protein with a KD of less than 40 nM (e.g., 35, 30, 25, 20, 15, 10, 9, 8, 7, 6, 5, 4, 3, 2, 1, 0.5, 0.25, 0.1 nM). In embodiments, the recombinant protein or chimeric antigen receptor binds to an ROR-1 protein with a KD of less than about 35 nM. In embodiments, the recombinant protein or chimeric antigen receptor binds to an ROR-1 protein with a KD of less than 35 nM. In embodiments, the recombinant protein or chimeric antigen receptor binds to an ROR-1 protein with a KD of less than about 30 nM. In embodiments, the recombinant protein or chimeric antigen receptor binds to an ROR-1 protein with a KD of less than 30 nM. In embodiments, the recombinant protein or chimeric antigen receptor binds to an ROR-1 protein with a KD of less than about 25 nM. In embodiments, the recombinant protein or chimeric antigen receptor binds to an ROR-1 protein with a KD of less than 25 nM. In embodiments, the recombinant protein or chimeric antigen receptor binds to an ROR-1 protein with a KD of less than about 20 nM. In embodiments, the recombinant protein or chimeric antigen receptor binds to an ROR-1 protein with a KD of less than 20 nM. In embodiments, the recombinant protein or chimeric antigen receptor binds to an ROR-1 protein with a KD of less than about 15 nM. In embodiments, the recombinant protein or chimeric antigen receptor binds to an ROR-1 protein with a KD of less than 15 nM. In embodiments, the recombinant protein or chimeric antigen receptor binds to an ROR-1 protein with a KD of less than about 10 nM. In embodiments, the recombinant protein or chimeric antigen receptor binds to an ROR-1 protein with a KD of less than 10 nM. In embodiments, the recombinant protein or chimeric antigen receptor binds to an ROR-1 protein with a KD of less than about 9 nM. In embodiments, the recombinant protein or chimeric antigen receptor binds to an ROR-1 protein with a KD of less than 9 nM. In embodiments, the recombinant protein or chimeric antigen receptor binds to an ROR-1 protein with a KD of less than about 8 nM. In embodiments, the recombinant protein or chimeric antigen receptor binds to an ROR-1 protein with a KD of less than 8 nM. In embodiments, the recombinant protein or chimeric antigen receptor binds to an ROR-1 protein with a KD of less than about 7 nM. In embodiments, the recombinant protein or chimeric antigen receptor binds to an ROR-1 protein with a KD of less than 7 nM. In embodiments, the recombinant protein or chimeric antigen receptor binds to an ROR-1 protein with a KD of less than about 6 nM. In embodiments, the recombinant protein or chimeric antigen receptor binds to an ROR-1 protein with a KD of less than 6 nM. In embodiments, the recombinant protein or chimeric antigen receptor binds to an ROR-1 protein with a KD of less than about 5 nM. In embodiments, the recombinant protein or chimeric antigen receptor binds to an ROR-1 protein with a KD of less than 5 nM. In embodiments, the recombinant protein or chimeric antigen receptor binds to an ROR-1 protein with a KD of less than about 4 nM. In embodiments, the recombinant protein or chimeric antigen receptor binds to an ROR-1 protein with a KD of less than 4 nM. In embodiments, the recombinant protein or chimeric antigen receptor binds to an ROR-1 protein with a KD of less than about 3 nM. In embodiments, the recombinant protein or chimeric antigen receptor binds to an ROR-1 protein with a KD of less than 3 nM. In embodiments, the recombinant protein or chimeric antigen receptor binds to an ROR-1 protein with a KD of less than about 2 nM. In embodiments, the recombinant protein or chimeric antigen receptor binds to an ROR-1 protein with a KD of less than 2 nM. In embodiments, the recombinant protein or chimeric antigen receptor binds to an ROR-1 protein with a KD of less than about 1 nM. In embodiments, the recombinant protein or chimeric antigen receptor binds to an ROR-1 protein with a KD of less than 1 nM. In embodiments, the recombinant protein or chimeric antigen receptor binds to an ROR-1 protein with a KD of less than about 0.5 nM. In embodiments, the recombinant protein or chimeric antigen receptor binds to an ROR-1 protein with a KD of less than 0.5 nM. In embodiments, the recombinant protein or chimeric antigen receptor binds to an ROR-1 protein with a KD of less than about 0.25 nM. In embodiments, the recombinant protein or chimeric antigen receptor binds to an ROR-1 protein with a KD of less than 0.25 nM. In embodiments, the recombinant protein or chimeric antigen receptor binds to an ROR-1 protein with a KD of less than about 0.1 nM. In embodiments, the recombinant protein or chimeric antigen receptor binds to an ROR-1 protein with a KD of less than 0.1 nM.

Further provided herein are recombinant nucleic acids encoding the recombinant protein or chimeric antigen receptor provided herein including embodiments thereof. In some embodiments, the chimeric antigen receptor is encoded by a single recombinant nucleic acid that forms part of an expression vector. Any suitable expression vector that is capable of being transfected into and expressed in an immune cells (e.g., CD8+ T cells, CD4+ T cells, CD56+ immune cells, NK cells, or genetically modified or engineered NK cells) are contemplated. Exemplary and/or preferred expression vectors may include a viral expression vector (e.g., expression vectors for adenovirus, adeno-associated viruses, alphaviruses, herpes viruses, lentiviruses, etc.). In some embodiments, an adenovirus is a replication deficient and non-immunogenic virus, which is typically accomplished by targeted deletion of selected viral proteins (e.g., E1, E2b, E3 proteins). For example, the recombinant nucleic acid can be placed in a non-viral vector (e.g., mammalian expression vector) and transfected to the T cells using any generally used transfection method. For other example, the recombinant nucleic acid in a viral vector such that the viral particle including recombinant nucleic acid infect the T cells to deliver the recombinant nucleic acid into the T cells. For still other example, where the recombinant nucleic acid is a self-replicating RNA-based vector, self-replicating RNA-based vector can be formulated with a pharmaceutically acceptable carrier (e.g., in a buffer or cell culture medium, preferably with RNAase inhibitors)) such that the self-replicating RNA-based vector can be delivered in a naked form to the cell by contacting directly to the cell membrane. In some embodiments, the self-replicating RNA-based vector can be coupled to a carrier molecule. Exemplary carrier molecules includes protein A, protein G, protein Z, albumin, refolded albumin, a nanoparticle (e.g., quantum dots, gold nanoparticles, magnetic nanoparticles, nanotubes, polymeric nanoparticles, dendrimers, etc.), or a bead (e.g., polystyrene bead, latex bead, dynabead, etc.). Preferably, the nanoparticle and/or beads have a dimension below 1 m, preferably below 100 nm. In other embodiments, the self-replicating RNA-based vector can be coupled with a micro particle (e.g., PLG RG503 (50:50 lactide/glycolide molar ratio), where the self-replicating RNA-based vector can be absorbed to the micro particle for further delivery to the cell. In still other embodiments, the self-replicating RNA-based vector can be encapsulated in a liposome (e.g., PEG-based liposome, etc.) to protect the self-replicating RNA-based vector from RNAase digestion and deliver the RNA-based vector by fusing the liposome to the target cell membrane.

In embodiments, the recombinant nucleic acids encoding the recombinant protein or chimeric antigen receptor provided herein can be inserted into a vector having a cassette for gene editing (e.g., CRISPR-CAS expression vector). Another exemplary expression vectors may include transposon-based expression system (e.g., Sleeping Beauty system, as disclosed in Deninger et al., PLOS ONE, Jun. 1, 2015).

### Cells and Pharmaceutical Compositions Thereof

Provided herein is a cell expressing a recombinant protein as described herein, which includes a chimeric antigen receptor (CAR) comprising a ROR-1-binding antibody or antibody fragment region, a linker domain, a transmembrane domain, and an intracellular domain. In embodiments, the ROR-1 binding domain is an antibody.

The chimeric antigen receptor described herein is expressed on the surface of T lymphocytes or T cells, such as, for example, CD8+ T cells and CD4+ T cells isolated from an individual afflicted with neuroendocrine cancer by genetically engineering the cells to express the heterologous nucleic acid sequences encoding the chimeric nucleic acid receptor. Such generated CAR-T cells are then re-administered to the individual. Once administered, the CAR-T cells specifically bind to ROR-1-expressing cells, such as neuroendocrine cancer cells expressing ROR-1, to elicit an immune response against the neuroendocrine cancer cells.

In embodiments, the chimeric antigen receptor described herein is expressed on the surface of natural killer cells isolated from an individual afflicted with neuroendocrine cancer and then genetically modified to express the ROR-1 CAR. In certain embodiments, the NK cells are CD56 positive cells. CD56 positivity can be determined by for example flow cytometry analysis of a cell population and defined as cells at least 10×, 100×, or 1,000× compared to cells stained with an isotype control antibody. In certain embodiments, the NK cells are primary NK cells. In embodiments, the NK cells comprise an NK cell line. In certain embodiments, the NK cells are autologous to an individual being treated with a CAR expressing NK cell of this disclosure. In embodiments, the NK cells are heterologous to an individual being treated with a CAR expressing NK cell of this disclosure. In embodiments, the NK cells are allogeneic to an individual being treated with a CAR expressing NK cell of this disclosure. NK cells can be derived from any suitable source bone marrow, induced pluripotent stem cells, peripheral blood mononuvlear cells, fetal or placental cells.

In embodiments, the cell is administered as part of a pharmaceutical composition. The pharmaceutical composition comprises a pharmaceutical acceptable excipient, diluent or carrier in addition to the cell that expresses the CAR comprising a ROR-1-binding antibody region.

The chimeric antigen receptor(s) and/or the recombinant nucleic acid encoding the chimeric antigen receptor(s) may be formulated as a pharmaceutical composition, optionally with any pharmaceutically acceptable carrier (e.g., as a sterile injectable composition for immune cells expressing chimeric antigen receptor(s), a pharmaceutically acceptable salt for recombinant nucleic acid encoding the chimeric antigen receptor(s), etc.). While the dose or cell titer of the pharmaceutical composition may vary depending on the treatment protocols, treatment regimens, treatment conditions, etc., one example of the dose or cell titer of the pharmaceutical composition may include a cell titer of at least 1×103 cells/ml, preferably at least 1×105 cells/ml, more preferably at least 1×106 cells/ml, and at least 1 ml, preferably at least 5 ml, more preferably and at least 20 ml per dosage unit.

In some embodiments, the pharmaceutical composition may include a homogenous cell or plurality thereof (e.g., CD8+ T cells expressing the chimeric antigen receptor, CD4+ T cells expressing the chimeric antigen receptor NK cells expressing the chimeric antigen receptor, etc.). In other embodiments, the composition can comprise a mixture of heterogeneous cells (e.g., mixture of CD8+ T cells expressing the chimeric antigen receptor and NK cells expressing the chimeric antigen receptor, etc., in a ratio of 1:1, 1:2, 1:3, 1:4, 4:1, 3:1, 2:1, etc.). The ratio of different types of cells may vary based on the type of neuroendocrine cancer, age, gender, or health status of the patient, size of tumor, and the cell counts of the patient. The actual amount effective for a particular application will depend, inter alia, on the condition being treated. When administered in methods to treat a disease, the recombinant proteins described herein will contain an amount of active ingredient effective to achieve the desired result, e.g., modulating the activity of a target molecule, and/or reducing, eliminating, or slowing the progression of disease symptoms. Determination of a therapeutically effective amount of a compound of the invention is well within the capabilities of those skilled in the art, especially in light of the detailed disclosure herein.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g., symptoms of cancer and severity of such symptoms), kind of concurrent treatment, complications from the disease being treated or other health-related problems. Other therapeutic regimens or agents can be used in conjunction with the methods and compounds of the invention. Adjustment and manipulation of established dosages (e.g., frequency and duration) are well within the ability of those skilled in the art.

For any composition (e.g., recombinant protein, nucleic acid) provided herein, the therapeutically effective amount can be initially determined from cell culture assays. Target concentrations will be those concentrations of active compound(s) that are capable of achieving the methods described herein, as measured using the methods described herein or known in the art. As is well known in the art, effective amounts for use in humans can also be determined from animal models. For example, a dose for humans can be formulated to achieve a concentration that has been found to be effective in animals. The dosage in humans can be adjusted by monitoring effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to affect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, and the like.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

### Methods of Treatment

The CAR-T cell compositions described herein are useful for treating a neuroendocrine cancer in an individual in need thereof, and for preventing or treating a metastasis originating from a neuroendocrine cancer.

Thus, described herein are methods of treating a neuroendocrine cancer and methods of preventing or treating metastasis originating from a neuroendocrine cancer, that comprise the administration of cells that express chimeric antigen receptors that target human ROR-1. The chimeric antigen receptors described herein are expressed by T lymphocytes or natural killer cells isolated from an individual afflicted with neuroendocrine cancer and re-administered to the individual. Administration of T cells expressing the CARs described herein serves as an effective therapeutic treatment for neuroendocrine cancers that express ROR-1 and metastases thereof.

The chimeric antigen receptors comprise i) an antigen binding region, wherein the antigen binding region specifically binds ROR-1 and wherein the antigen binding region comprises a light chain variable domain and a heavy chain variable domain, ii) a spacer domain, iii) a transmembrane domain, and iv) an intracellular domain. The light chain variable domain comprises a CDR L1 as set forth in SEQ ID NO:43, a CDR L2 as set forth in SEQ ID NO:44 and a CDR L3 as set forth in SEQ ID NO:45, and the heavy chain variable domain comprises a CDR H1 as set forth in SEQ ID NO:46, a CDR H2 as set forth in SEQ ID NO:47, and a CDR H3 as set forth in SEQ ID NO:48. Alternatively, the light chain variable domain comprises a CDR L1 as set forth in SEQ ID NO:49, a CDR L2 as set forth in SEQ ID NO:50 and a CDR L3 as set forth in SEQ ID NO:51, and the heavy chain variable domain comprises a CDR H1 as set forth in SEQ ID NO:52, a CDR H2 as set forth in SEQ ID NO:53, and a CDR H3 as set forth in SEQ ID NO:54.

In some instances, the light chain variable domain is covalently linked to the heavy chain variable domain through a polypeptide linker. In some embodiments, the polypeptide linker comprises an amino acid sequence of SEQ ID NO: 24.

In embodiments, the spacer domain comprises an antibody domain. In some embodiments, the antibody domain comprises an immunoglobulin hinge domain, an immunoglobulin constant heavy chain 3 (CH3) domain, an immunoglobulin constant heavy chain 2 (CH2) domain, or a combination thereof.

In embodiments, the spacer domain comprises an amino acid sequence represented by SEQ ID NO: 29, SEQ ID NO: 41 or SEQ ID NO: 42.

In embodiments, the light chain variable domain comprises an amino acid sequence at least about 90%, 95%, 97%, 98%, 99%, or 100% identical to SEQ ID NO: 21.

In embodiments, the heavy chain variable domain comprises an amino acid sequence at least about 90%, 95%, 97%, 98%, 99%, or 100% identical to SEQ ID NO: 27.

In embodiments, the transmembrane domain comprises a CD8α transmembrane domain, a CD28 transmembrane domain, a CD4 transmembrane domain, a CD3ζ transmembrane domain, or any combination thereof. In some aspects, the transmembrane domain is a CD28 transmembrane domain.

In embodiments, the intracellular domain comprises an intracellular co-stimulatory signaling domain, an intracellular T-cell signaling domain, or a combination thereof. The intracellular co-stimulatory signaling domain is a 4-1BB intracellular co-stimulatory signaling domain, a CD28 intracellular co-stimulatory signaling domain, a ICOS intracellular co-stimulatory signaling domain, an OX-40 intracellular co-stimulatory signaling domain, or any combination thereof. In some embodiments, the intracellular costimulatory signaling domain comprises a 41-BB intracellular co-stimulatory signaling domain. In other embodiments, the intracellular costimulatory signaling domain comprises a CD28 intracellular co-stimulatory signaling domain and a 41-BB intracellular co-stimulatory signaling domain.

In embodiments, the intracellular costimulatory signaling domain further comprises an intracellular T-cell signaling domain. In some embodiments, the intracellular T-cell signaling domain is a CD3ζ intracellular T-cell signaling domain.

In embodiments, the chimeric antigen receptor binds to a cell expressing ROR-1. In some embodiments, the cell expressing ROR-1 is a neuroendocrine cancerous cell.

In embodiments, the cell is a T lymphocyte. In some embodiments, the T lymphocyte is a CD4+T lymphocyte or a CD8+T lymphocyte.

In embodiments, the cell is a natural killer cell. In some embodiments, the natural killer cell is autologous to the subject. In other embodiments, the natural killer cell is heterologous to the subject. In yet other embodiments, the natural killer cell is allogeneic to the subject.

Neuroendocrine cancers that may be treated by the methods provided herein include, but are not limited to, carcinoid tumors, islet cell tumors, medullary thyroid cancers, pheochromocytoma, neuroendocrine carcinoma of the skin, small cell lung cancer, large cell neuroendocrine carcinoma, neuroendocrine prostate cancer (NEPC), and metastatic castration-resistant prostate cancer (CRPC). In some aspects, the neuroendocrine cancer is metastatic castration-resistant prostate cancer (CRPC).

In embodiments, the neuroendocrine cancerous cells express ROR-1. In some instances, the subject has failed to respond to androgen deprivation therapy. In some instances, the neuroendocrine cancer has metastasized to bone.

In embodiments, the methods provided herein further comprise administering cirmtuzumab to the subject. In some embodiments, the cirmtuzumab and the cells expressing the disclosed chimeric antigen receptors are administered separately. In other embodiments, the cirmtuzumab and the cells are administered together.

In embodiments, the methods provided herein further comprise administering to the subject platinum-based chemotherapy. Suitable platinum-based chemotherapy includes, but it is not limited to, carboplatin, cisplatin, etoposide, a taxane, and any combination thereof.

Treatment refers to a method that seeks to improve or ameliorate the condition being treated. With respect to neuroendocrine cancer, treatment includes, but is not limited to, reduction of tumor volume, reduction in growth of tumor volume, increase in progression-free survival, or overall life expectancy. In certain embodiments, treatment will effect remission of a neuroendocrine cancer being treated. In certain embodiments, treatment encompasses use as a prophylactic or maintenance dose intended to prevent reoccurrence or progression of a previously treated neuroendocrine cancer or tumor. It is understood by those of skill in the art that not all individuals will respond equally or at all to a treatment that is administered, nevertheless these individuals are considered to be treated.

The anti-ROR-1 CAR T-cells and anti-ROR-1 CAR-T cell compositions can be administered to a subject in need thereof by any route suitable for the administration of cell-containing pharmaceutical compositions, such as, for example, subcutaneous, intraperitoneal, intravenous, intramuscular, intratumoral, or intracerebral, etc. In certain embodiments, the antibodies are administered intravenously. In certain embodiments, the antibodies are administered subcutaneously. In certain embodiments, the antibodies are administered intratumorally.

The anti-ROR-1 CAR T-cells and anti-ROR-1 CAR-T cell compositions can be administered according to a suitable dosage schedule. In certain embodiments, the CAR T-cells are administered once, with subsequent doses depending on clinical criteria. If an individual does not respond or only partially responds said patient can have an anti-ROR-1 CAR-T cell composition administered a second, third, or fourth time until the desired clinical response is observed. A dosage of CAR-T cells will generally comprise at least 1×106 cells, but no more than 5×108 cells. Cells can be administered based upon a total amount of an individual's viable PBMC that were transduced with a CAR construct. In certain embodiments, a single dosage comprises 1 million transduced PBMCs to 100 million transduced PBMCs. In certain embodiments, a single dosage comprises 1 million transduced PBMCs to 2 million transduced PBMCs, 1 million transduced PBMCs to 3 million transduced PBMCs, 1 million transduced PBMCs to 4 million transduced PBMCs, 1 million transduced PBMCs to 5 million transduced PBMCs, 1 million transduced PBMCs to 6 million transduced PBMCs, 1 million transduced PBMCs to 7 million transduced PBMCs, 1 million transduced PBMCs to 8 million transduced PBMCs, 1 million transduced PBMCs to 9 million transduced PBMCs, 1 million transduced PBMCs to 10 million transduced PBMCs, 1 million transduced PBMCs to 50 million transduced PBMCs, 1 million transduced PBMCs to 100 million transduced PBMCs, 2 million transduced PBMCs to 3 million transduced PBMCs, 2 million transduced PBMCs to 4 million transduced PBMCs, 2 million transduced PBMCs to 5 million transduced PBMCs, 2 million transduced PBMCs to 6 million transduced PBMCs, 2 million transduced PBMCs to 7 million transduced PBMCs, 2 million transduced PBMCs to 8 million transduced PBMCs, 2 million transduced PBMCs to 9 million transduced PBMCs, 2 million transduced PBMCs to 10 million transduced PBMCs, 2 million transduced PBMCs to 50 million transduced PBMCs, 2 million transduced PBMCs to 100 million transduced PBMCs, 3 million transduced PBMCs to 4 million transduced PBMCs, 3 million transduced PBMCs to 5 million transduced PBMCs, 3 million transduced PBMCs to 6 million transduced PBMCs, 3 million transduced PBMCs to 7 million transduced PBMCs, 3 million transduced PBMCs to 8 million transduced PBMCs, 3 million transduced PBMCs to 9 million transduced PBMCs, 3 million transduced PBMCs to 10 million transduced PBMCs, 3 million transduced PBMCs to 50 million transduced PBMCs, 3 million transduced PBMCs to 100 million transduced PBMCs, 4 million transduced PBMCs to 5 million transduced PBMCs, 4 million transduced PBMCs to 6 million transduced PBMCs, 4 million transduced PBMCs to 7 million transduced PBMCs, 4 million transduced PBMCs to 8 million transduced PBMCs, 4 million transduced PBMCs to 9 million transduced PBMCs, 4 million transduced PBMCs to 10 million transduced PBMCs, 4 million transduced PBMCs to 50 million transduced PBMCs, 4 million transduced PBMCs to 100 million transduced PBMCs, 5 million transduced PBMCs to 6 million transduced PBMCs, 5 million transduced PBMCs to 7 million transduced PBMCs, 5 million transduced PBMCs to 8 million transduced PBMCs, 5 million transduced PBMCs to 9 million transduced PBMCs, 5 million transduced PBMCs to 10 million transduced PBMCs, 5 million transduced PBMCs to 50 million transduced PBMCs, 5 million transduced PBMCs to 100 million transduced PBMCs, 6 million transduced PBMCs to 7 million transduced PBMCs, 6 million transduced PBMCs to 8 million transduced PBMCs, 6 million transduced PBMCs to 9 million transduced PBMCs, 6 million transduced PBMCs to 10 million transduced PBMCs, 6 million transduced PBMCs to 50 million transduced PBMCs, 6 million transduced PBMCs to 100 million transduced PBMCs, 7 million transduced PBMCs to 8 million transduced PBMCs, 7 million transduced PBMCs to 9 million transduced PBMCs, 7 million transduced PBMCs to 10 million transduced PBMCs, 7 million transduced PBMCs to 50 million transduced PBMCs, 7 million transduced PBMCs to 100 million transduced PBMCs, 8 million transduced PBMCs to 9 million transduced PBMCs, 8 million transduced PBMCs to 10 million transduced PBMCs, 8 million transduced PBMCs to 50 million transduced PBMCs, 8 million transduced PBMCs to 100 million transduced PBMCs, 9 million transduced PBMCs to 10 million transduced PBMCs, 9 million transduced PBMCs to 50 million transduced PBMCs, 9 million transduced PBMCs to 100 million transduced PBMCs, 10 million transduced PBMCs to 50 million transduced PBMCs, 10 million transduced PBMCs to 100 million transduced PBMCs, or 50 million transduced PBMCs to 100 million transduced PBMCs. In certain embodiments, a single dosage comprises 1 million transduced PBMCs, 2 million transduced PBMCs, 3 million transduced PBMCs, 4 million transduced PBMCs, 5 million transduced PBMCs, 6 million transduced PBMCs, 7 million transduced PBMCs, 8 million transduced PBMCs, 9 million transduced PBMCs, 10 million transduced PBMCs, 50 million transduced PBMCs, or 100 million transduced PBMCs. In certain embodiments, a single dosage comprises at least 1 million transduced PBMCs, 2 million transduced PBMCs, 3 million transduced PBMCs, 4 million transduced PBMCs, 5 million transduced PBMCs, 6 million transduced PBMCs, 7 million transduced PBMCs, 8 million transduced PBMCs, 9 million transduced PBMCs, 10 million transduced PBMCs, or 50 million transduced PBMCs. In certain embodiments, a single dosage comprises at most 2 million transduced PBMCs, 3 million transduced PBMCs, 4 million transduced PBMCs, 5 million transduced PBMCs, 6 million transduced PBMCs, 7 million transduced PBMCs, 8 million transduced PBMCs, 9 million transduced PBMCs, 10 million transduced PBMCs, 50 million transduced PBMCs, or 100 million transduced PBMCs.

More or less cells may be used depending on the transduction efficiency of an individual's T cells on a case by case basis. In certain embodiments, a single dosage comprises 1 million CAR-T cells to 100 million CAR-T cells. In certain embodiments, a single dosage comprises 1 million CAR-T cells to 2 million CAR-T cells, 1 million CAR-T cells to 3 million CAR-T cells, 1 million CAR-T cells to 4 million CAR-T cells, 1 million CAR-T cells to 5 million CAR-T cells, 1 million CAR-T cells to 6 million CAR-T cells, 1 million CAR-T cells to 7 million CAR-T cells, 1 million CAR-T cells to 8 million CAR-T cells, 1 million CAR-T cells to 9 million CAR-T cells, 1 million CAR-T cells to 10 million CAR-T cells, 1 million CAR-T cells to 50 million CAR-T cells, 1 million CAR-T cells to 100 million CAR-T cells, 2 million CAR-T cells to 3 million CAR-T cells, 2 million CAR-T cells to 4 million CAR-T cells, 2 million CAR-T cells to 5 million CAR-T cells, 2 million CAR-T cells to 6 million CAR-T cells, 2 million CAR-T cells to 7 million CAR-T cells, 2 million CAR-T cells to 8 million CAR-T cells, 2 million CAR-T cells to 9 million CAR-T cells, 2 million CAR-T cells to 10 million CAR-T cells, 2 million CAR-T cells to 50 million CAR-T cells, 2 million CAR-T cells to 100 million CAR-T cells, 3 million CAR-T cells to 4 million CAR-T cells, 3 million CAR-T cells to 5 million CAR-T cells, 3 million CAR-T cells to 6 million CAR-T cells, 3 million CAR-T cells to 7 million CAR-T cells, 3 million CAR-T cells to 8 million CAR-T cells, 3 million CAR-T cells to 9 million CAR-T cells, 3 million CAR-T cells to 10 million CAR-T cells, 3 million CAR-T cells to 50 million CAR-T cells, 3 million CAR-T cells to 100 million CAR-T cells, 4 million CAR-T cells to 5 million CAR-T cells, 4 million CAR-T cells to 6 million CAR-T cells, 4 million CAR-T cells to 7 million CAR-T cells, 4 million CAR-T cells to 8 million CAR-T cells, 4 million CAR-T cells to 9 million CAR-T cells, 4 million CAR-T cells to 10 million CAR-T cells, 4 million CAR-T cells to 50 million CAR-T cells, 4 million CAR-T cells to 100 million CAR-T cells, 5 million CAR-T cells to 6 million CAR-T cells, 5 million CAR-T cells to 7 million CAR-T cells, 5 million CAR-T cells to 8 million CAR-T cells, 5 million CAR-T cells to 9 million CAR-T cells, 5 million CAR-T cells to 10 million CAR-T cells, 5 million CAR-T cells to 50 million CAR-T cells, 5 million CAR-T cells to 100 million CAR-T cells, 6 million CAR-T cells to 7 million CAR-T cells, 6 million CAR-T cells to 8 million CAR-T cells, 6 million CAR-T cells to 9 million CAR-T cells, 6 million CAR-T cells to 10 million CAR-T cells, 6 million CAR-T cells to 50 million CAR-T cells, 6 million CAR-T cells to 100 million CAR-T cells, 7 million CAR-T cells to 8 million CAR-T cells, 7 million CAR-T cells to 9 million CAR-T cells, 7 million CAR-T cells to 10 million CAR-T cells, 7 million CAR-T cells to 50 million CAR-T cells, 7 million CAR-T cells to 100 million CAR-T cells, 8 million CAR-T cells to 9 million CAR-T cells, 8 million CAR-T cells to 10 million CAR-T cells, 8 million CAR-T cells to 50 million CAR-T cells, 8 million CAR-T cells to 100 million CAR-T cells, 9 million CAR-T cells to 10 million CAR-T cells, 9 million CAR-T cells to 50 million CAR-T cells, 9 million CAR-T cells to 100 million CAR-T cells, 10 million CAR-T cells to 50 million CAR-T cells, 10 million CAR-T cells to 100 million CAR-T cells, or 50 million CAR-T cells to 100 million CAR-T cells. In certain embodiments, a single dosage comprises 1 million CAR-T cells, 2 million CAR-T cells, 3 million CAR-T cells, 4 million CAR-T cells, 5 million CAR-T cells, 6 million CAR-T cells, 7 million CAR-T cells, 8 million CAR-T cells, 9 million CAR-T cells, 10 million CAR-T cells, 50 million CAR-T cells, or 100 million CAR-T cells. In certain embodiments, a single dosage comprises at least 1 million CAR-T cells, 2 million CAR-T cells, 3 million CAR-T cells, 4 million CAR-T cells, 5 million CAR-T cells, 6 million CAR-T cells, 7 million CAR-T cells, 8 million CAR-T cells, 9 million CAR-T cells, 10 million CAR-T cells, or 50 million CAR-T cells. In certain embodiments, a single dosage comprises at most 2 million CAR-T cells, 3 million CAR-T cells, 4 million CAR-T cells, 5 million CAR-T cells, 6 million CAR-T cells, 7 million CAR-T cells, 8 million CAR-T cells, 9 million CAR-T cells, 10 million CAR-T cells, 50 million CAR-T cells, or 100 million CAR-T cells.

In certain embodiments, a population of the anti-ROR-1 CAR-T cells of the current disclosure are included in a pharmaceutical composition comprising one or more pharmaceutically acceptable excipients, carriers, and diluents. In certain embodiments, the CAR-T cells of the current disclosure are administered suspended in a sterile isotonic solution. In certain embodiments, the solution comprises about 0.9% NaCl. In certain embodiments, the solution comprises about 5.0% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA. The CAR-T cells when formulated, can be buffered at a certain pH, generally between about 7.0 and about 8.0. In certain embodiments, the cells are buffered at a physiological pH of about 7.4 (averaging between about 7.35 and 7.45.

Also described herein are methods of treating an individual that has developed one or more serious adverse events associated with CAR T-cell treatment comprising administering a bolus injection of cirmtuzumab. Such an administration blocks CAR T-cells from interacting with their targets and can attenuate their activity.

### EXAMPLES

The following illustrative examples are representative of embodiments of compositions and methods described herein and are not meant to be Limiting in any Way.

### Example 1: Chimeric Antigen Receptor Modified T-Cells (CAR-T) that Target Neuroendocrine Cancer Cells Expressing ROR-1

Pre-clinical studies involving the use of anti-human ROR1 T-cell CARS against a wide variety of solid and liquid tumor cancers have been previously described. However, when used clinically (NCT02706392 and NCT02194374), these same ROR1 CARS had minimal activity in treating patient ROR1^{pos} hematological and solid tumor cancers. The lack of therapeutic effect in these studies is due to the use of T-cell CARS comprising rabbit/human chimeric ROR1 targeting domains. These domains present the potential for off target binding, are recognized as foreign antigens by the immune system, and are therefore rapidly inactivated.

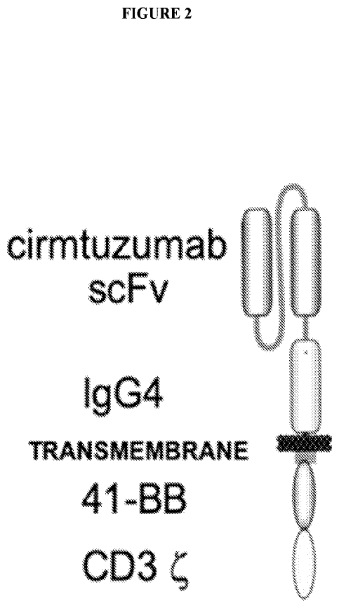

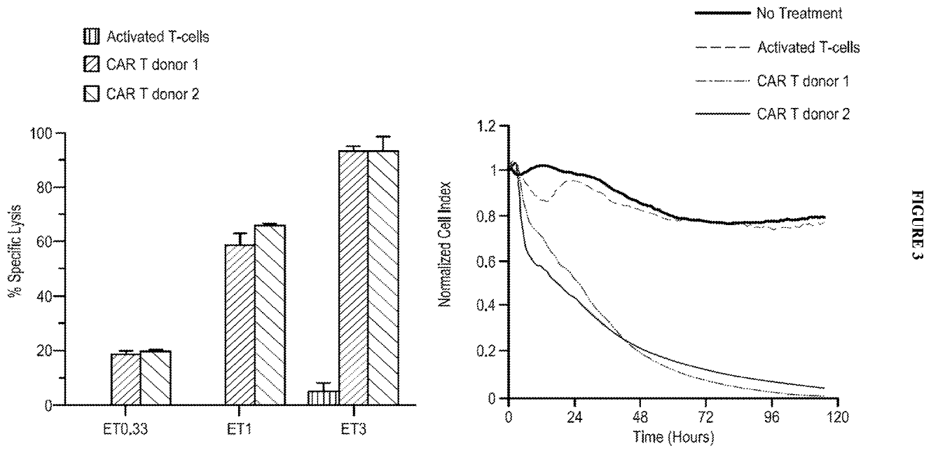

The anti-ROR1 T-cell CARs constructed and tested in these examples employ a scFv antigen targeting domain that is generated from fully humanized cirmtuzumab (FIG. 2), and which comprises the complementarity determining regions (CDRs) and variable regions (V_{H}/V_{L}) of the cirmtuzumab antibody. The examples presented herein show that T-cell CAR employing cirmtuzumab as the antigen binding component are effective in treating ROR1^{pos} neuroendocrine cancers that are resistant to other therapies.

### Treatment Failures

The use of potent therapies aimed at inhibiting critical oncogenic pathways active in epithelial cancers has led to multiple resistance mechanisms, including the development of highly aggressive neuroendocrine cancers, such as small cell neuroendocrine carcinoma (SCNC). SCNC can arise from almost all epithelial organs including the prostate and lung. Metastatic castration resistant prostate cancer (CRPC) is one of several lethal neuroendocrine cancers. Small cell prostate cancer (SCPC) is a type of neuroendocrine prostate cancer (NEPC), a particularly malignant form of CRPC.

One in six men is diagnosed with prostate cancer (PCa), and up to one quarter of PCa patients develop advanced prostate cancer with poor prognosis and five-year survival. The main treatment for these types of prostate cancers is androgen deprivation therapy (ADT) which targets androgen receptor (AR) signaling. Inevitably, however, the cancer progresses to ADT resistance and patients develop lethal castration resistant prostate cancer (CRPC) and metastasis. NEPC is emerging with increasing frequency in patients treated with ADT.

Most PCa metastasize to bone where it typically develops therapy resistance and there is no cure. ROR1 is an attractive therapeutic target, since it is expressed on a number of highly malignant hematological and solid tumor cancer cells, including CRPC and NEPC cells. However, despite the recent development of several novel therapies that have improved survival for patients, nearly all individuals with CRPC develop resistance to therapy and disease progression.

### Innovation

Because mCRPC and NEPC remain an urgent un-met medical need, we have produced a series of 2nd generation T-cell CAR constructs that when transduced into human T-cells demonstrated highly potent and specific anti-neuroendocrine tumoral activity and specificity in in vitro and in vivo test systems. As shown in FIG. 2, the CARs of this disclosure include light chain CDRs of the anti-human ROR-1 mAbs 4A5 or UC-961 attached to the heavy chain CDRs of the same mAbs by specific linkers that generate high-affinity single-chain (scFv) molecules that specifically bind human ROR-1 with a sufficient affinity to activate intracellular signaling and cytotoxicity in the transduced T-lymphocytes. To attach the scFv to a transmembrane domain, a series of protein spacers generated from IgG4 have been created. These spacers allow the anti-ROR-1 scFv binding domain sufficient flexibility to optimally bind the target ROR-1 antigen.

The extracellular antigen binding domain from the CARs is bound to a CD28 transmembrane domain. This transmembrane domain is then attached to an intracellular activating domain derived from CD28 and/or CD137, which are employed singularly (2^{nd}-gen CAR) or in combination (3^{rd}-gen CAR). These activating domains are then attached to the T-cell receptor activating domain contained within the CD3 zeta chain (CD3 ζ-chain). Therefore, the anti-ROR-1 CARs constructed and tested in these examples are expressed as a single polypeptide that from the N-terminal end contains in order: a leader that directs the construct to the cell surface, the light chain CDR of either the 4A5 or UC-961 mAbs, a linker that attaches the light and heavy chains, the heavy chain CDR of 4A5 or UC-961 mAbs that are complimented by the corresponding light chain molecules, a spacer of defined length generated from IgG4 including the IgG4 hinge region, the CD28 transmembrane region, the intracellular activation domains of CD28 and 41-BB (CD137)-individually or in tandem attached to the T-cell ζ-chain which extends to the carboxy terminus of the molecule. Employing clinical grade procedures and processes, we have now produced anti-ROR1 T-cell CAR products from over 20 normal human donor and chronic lymphocytic leukemia (CLL) patient products from subjects ranging in age from 31 to 83.

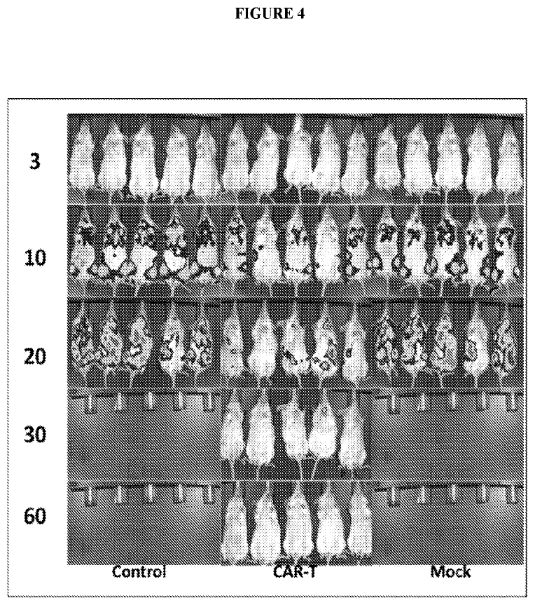

FIG. 3 present graphs showing in vitro cell killing activity of ROR1 CAR T-cells in a 4 h chromium release assay (left panel) and 120 h ACEA impedance assay (right panel) from T cells of two healthy donors, that were transduced with ROR1 at the indicated effect to target (E:T) ratios against leukemic Mec ROR1pos cells in the left panel and an ACEA impedance assay against MB 231 ROR1pos breast cancer cells in the right panel. The anti-ROR1 CAR T-cells demonstrated high and specific cytotoxicity without significant killing of ROR1-negative target cells. In in vitro studies, both the normal and patient derived T-cell CAR products demonstrated dose dependent activity against a wide-variety of ROR1pos solid and liquid tumor cell lines with little off-target activity (FIG. 3).

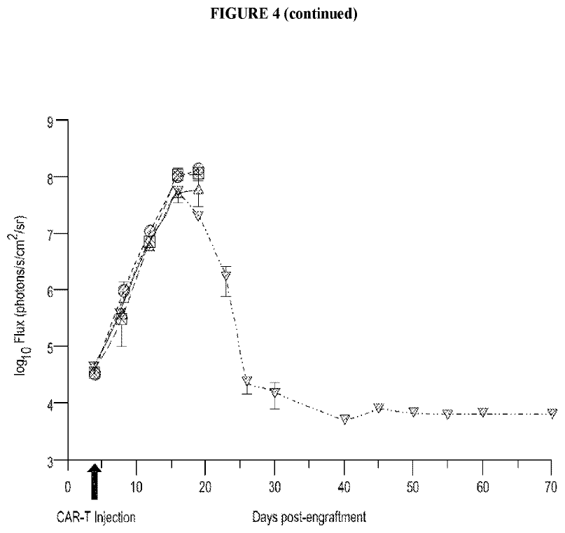

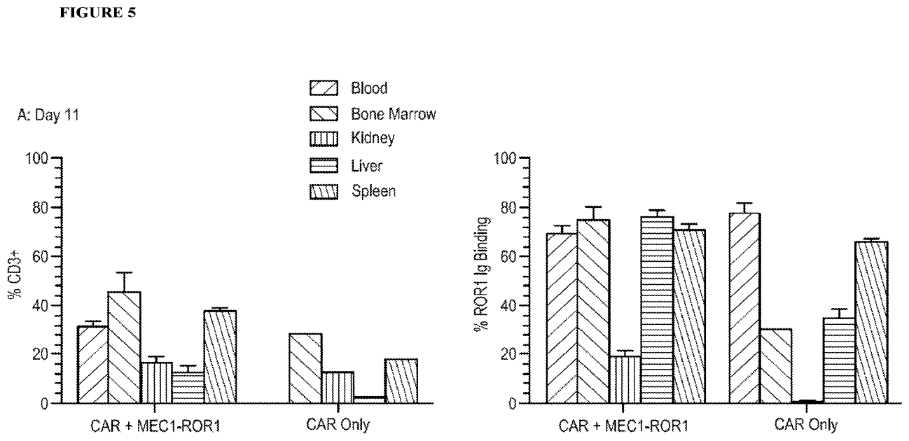

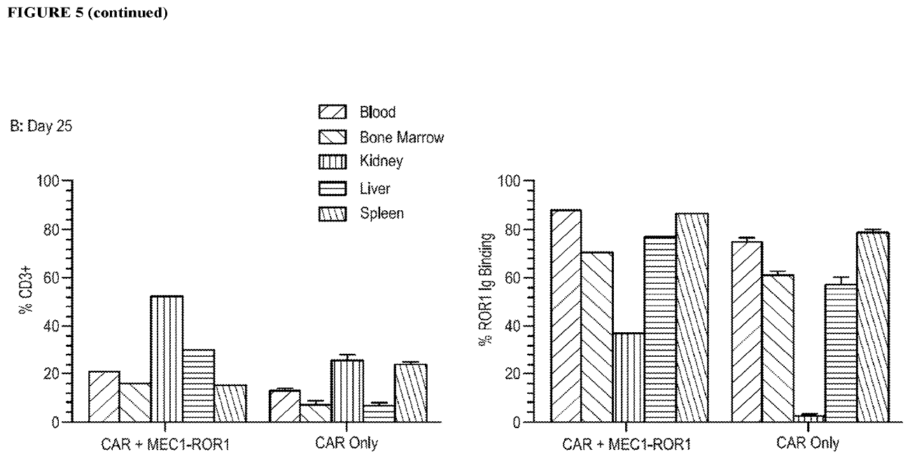

To confirm the activity of these products, we have also tested our anti-ROR1 T-cell CARs against a murine leukemia model that employed a human ROR1^{pos} CLL derived cells as targets (FIGS. 4 and 5).

FIG. 4 shows bioluminescence imaging of mice inoculated with MEC1-ROR1 cells and with ROR1 CAR T-Cells. Animals treated with CAR-T cells had reduced disease burden compared to controls. The highest dose (3×10⁶ CAR-T cells) cohort showed reduction of the leukemic burden to background levels by day 30, and had only minimal amounts of disease for the duration of the study. Animals in the control groups (untreated, mock transduced) had to be sacrificed on day 20. The right panel shows the total bioluminescent product collected from the mice. Blue square and circle are controls and green triangles ROR1-CAR T treated mice.

FIG. 5 shows ROR-1 CAR T cell expression in mouse tissues at various times. Following ROR-1 CAR-T administration, animals were sacrificed on days 11 (top panels) and day 25 (bottom panels). Blood and organs were collected and subjected to flow analysis for CAR expression and confirmatory ROR1 binding activity. The ROR-1 CAR-T cell number was substantially greater in mice bearing MEC-1 ROR1 cells (CAR+MEC1-ROR-1) vs control (CAR only), demonstrating elevated expansion of ROR-1 CAR-T cells in animals with tumor burden. Bars represent the mean values from the five mice in each group and error bars represent the S.E of the mean.

These results indicated that cirmtuzumab-based anti-ROR1 T cell CARS are highly potent and specific against tumor cells.

The CARs described herein can be included in a transposon-based or lentiviral vector, and introduced into target lymphocytes or natural killer cells employing standard transfection or transduction techniques. To maximize stable transduction and long-term expression of the CAR product, retroviral delivery systems are used including murine gamma and human lentivirus. Currently, a third generation lentiviral, four plasmid system (Addgene, Inc.) is used to render T lymphocytes transgenic for an ROR-1 expressing CAR. This third-generation system is based on the lentiviral vector system described in Naldini et al, “Efficient transfer, integration, and sustained long-term expression of the transgene in adult rat brains injected with a lentiviral vector” Proc Natl Acad Sci USA. 1996 Oct. 15; 93(21): 11382-11388. This four-plasmid system comprises: plasmid 1-gag/pol; plasmid 2-rev; plasmid 3 VSV-G protein; and plasmid 4, the transfer plasmid, which comprises a nucleic acid sequence encoding an anti-ROR1 CAR, which has been inserted using the restriction sites in the poly-linker.

### Example 2: ROR-1 is Expressed in Primary Human Prostate Cancer Cells

A study aimed at elucidating the expression of ROR1 in human neuroendocrine cancers including prostate cancer was performed.

### Tissue Microarray Analysis

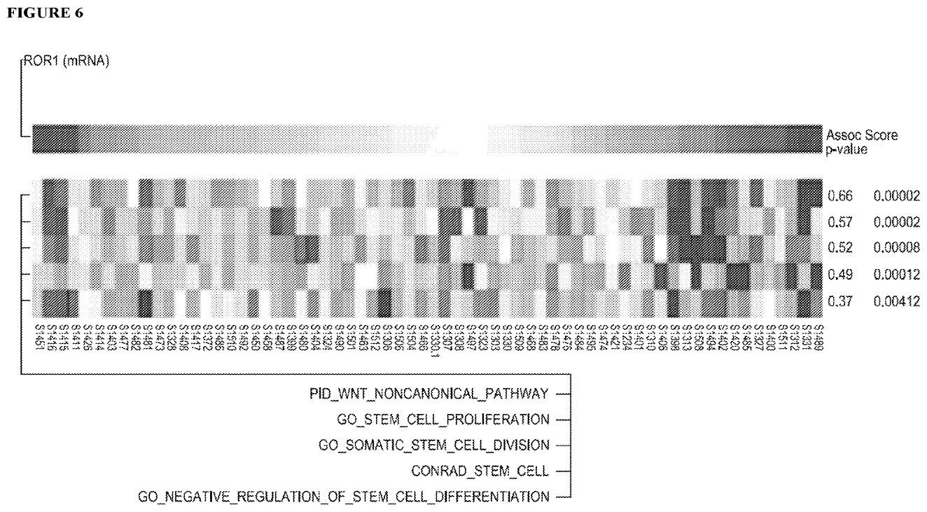

Tissue microarray evaluation revealed that ninety percent (n=19/21) of prostate cancers had moderate to strong staining for ROR1 on the plasma membrane (Table 1). Additionally, expression was associated with AKT activation and silencing of ROR1 expression impaired cell growth in vitro. FIG. 6 shows the results obtained from the analysis of tumor RNA sequences obtained from 66 prostate cancer samples, expressed as association of single-sample Gene Set Enrichment Analysis profiles of non-canonical WNT and stem cell gene sets with the expression of ROR1 (mRNA). The analysis shows a significant enrichment of WNT non-canonical and stem cell gene sets against ROR1 mRNA expression

### Prostate Cancer Patient-Derived Xenograft Models

Despite the challenges presented by PCa bone metastases, which are not typically surgically removed or biopsied, we established a Biobank and new patient-derived xenografts (PDX) and organoid models, which are used as pre-clinical models to accurately represent the patient disease and perform drug testing predictive of the patient response and drug efficacy.

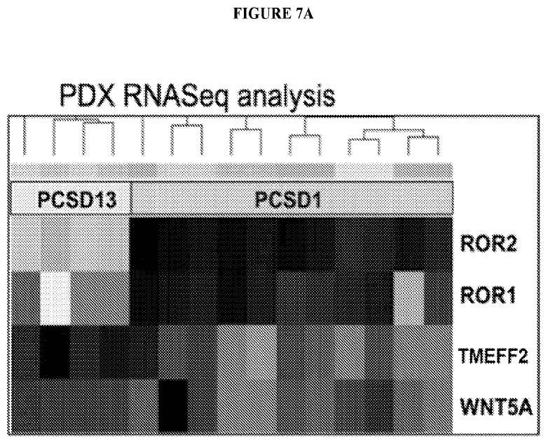

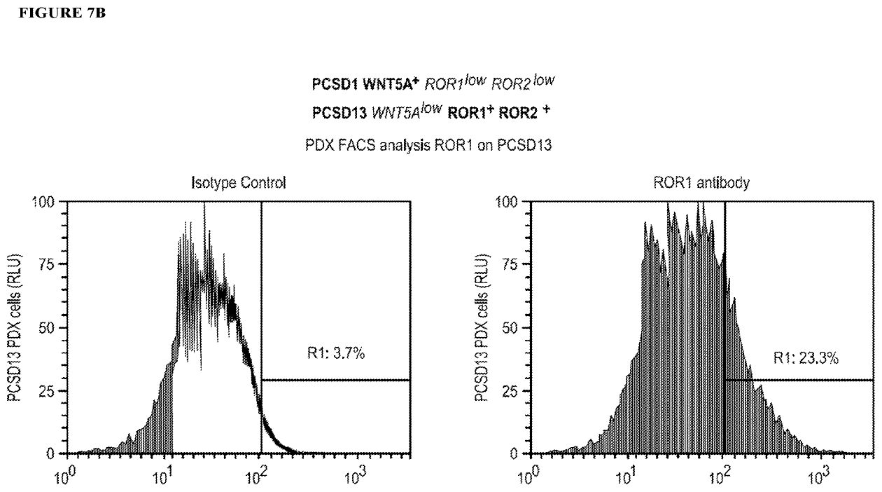

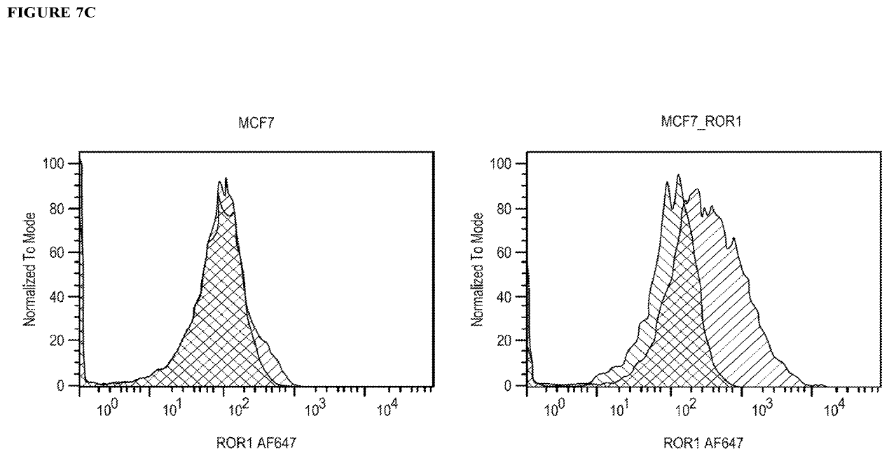

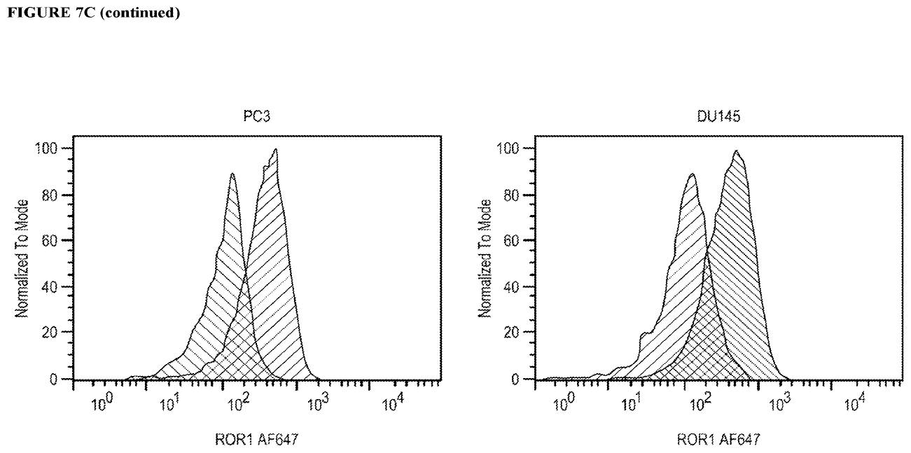

FIG. 4 shows the results obtained from whole human genome HTA2.0 Affymetrix microarray and RNASeq gene expression profiling. These results demonstrate that Wnt5A was highly expressed in a patient prostatic adenocarcinoma bone metastasis specimen and in its corresponding PDX, PCSD1. FIG. 7A shows that RNASeq analysis of another patient PCa bone metastasis-derived PDX, PCSD13, a small cell prostate cancer, showed significant ROR1 expression (FIG. 4). These data are indicative of the interplay between Wnt5A, ROR-1, ROR-2, and bone metastatic prostate cancer, as advanced CRPC is associated with Wnt5A and ROR1 expression.

FACS analysis revealed that ROR1 protein was highly expressed in PCSD13 cells as well as in PC3 and DU145 PCa cell lines (FIG. 7B). We noted the reciprocal expression of Wnt5a and ROR1 in the bone metastases from the prostate adenocarcinoma, PCSD1, compared to small cell prostate cancer, PCSD13. While PCSD1, was PSMA+WNT5A+ROR1^{low}ROR2^{low}, PCSD13, was PSMA⁻WNT5A⁻ROR1+ROR2+. This may reflect a fundamental difference in prostate adenocarcinoma and prostate small cell carcinoma, or NEPC. Our findings underscore the importance of ROR1 expression characterization at differential stages in prostate cancer to better understand the role of ROR1 in prostate cancer disease progression and the emergence of neuroendocrine prostate cancer (NEPC), and identify the most suitable patient population for ROR1 targeting with cirmtuzumab-CART cells.

### PDX-Derived Organoid Cultures

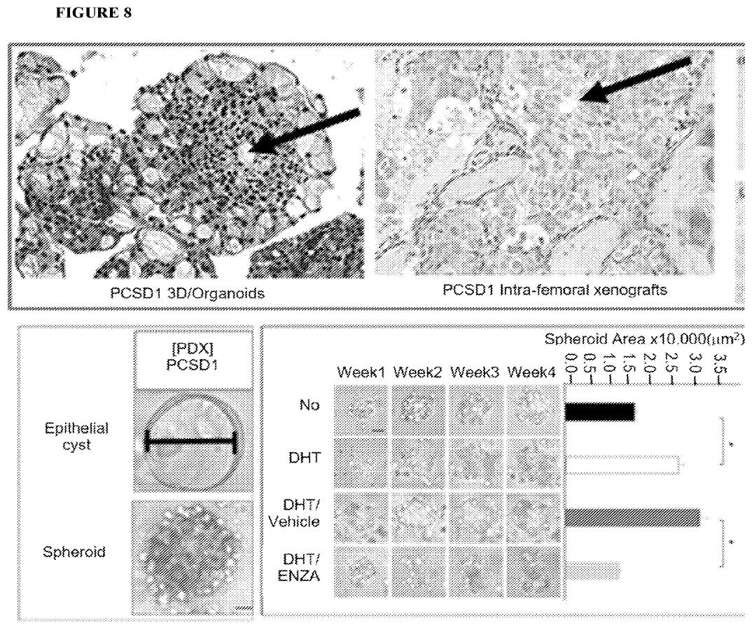

Three-dimensional (3D) organoid cell cultures from patient-derived xenograft (PDX) and primary patient tumor cells were established. FIG. 8 shows PCSD1 PDX tumors cells from three-dimensional organoids that replicate histomorphology of the xenografts growing in the femur bone. The 3D cultures consisted of two main cell masses: spheroids and epithelial cysts similar to gland-like structures seen in sections from xenografts growing within the femur bone displacing bone marrow and in the patient. Spheroids were resistant to androgen deprivation-induced death. Spheroid masses contained cyst-like structures surrounded by tumor cells that were similar to gland-like structures. Black Arrows point to the cyst structures. Organoids are GFP+. PDXs were obtained from prostate cancer bone metastases. PCSD1 expressing GFP-Luciferase formed different morphologies under 3D-culture conditions. FIG. 8 also shows that enzalutamide treatment of PCSD1 reduced lumen size and number of epithelial cysts. Transcriptome analysis showed that AR-responsive genes such as PSA (KLK3) and TMPRSS2 were downregulated under ADT while stem-cell transcription factors, steroidogenic and neurogenic pathway genes were upregulated. Thus, anti-androgens could still suppress canonical AR-responsive genes, but the tumor and organoid growth were resistant to ADT including the anti-androgen, enzalutamide.

### Example 3: Anti-ROR-1 T-Cell Cars are Highly Potent in Targeting and Killing Neuroendocrine Cancer Cell Lines Expressing ROR-1

PCa is highly heterogenous, exhibits a high recurrence rate following standard treatments and therefore, new therapeutic approaches are needed. We have generated a cirmtuzumab-based T-cell CAR to target treatment resistant ROR1^{pos} cancers. To test and demonstrate the activity of this CAR product, we have produced a series of 2^{d} generation T-cell CAR constructs that when transduced into human T-cells demonstrated highly potent and specific anti-tumoral activity and specificity in in vitro and in vivo test systems of hematological and human solid tumor cancers.

The aim of these studies is to determine the efficacy of human ROR1 CAR-T cells against PCa cell lines, PDX models and organoids of mCRPC and NEPC in combination with standard-of-care therapies including androgen deprivation therapy, and docetaxel, to determine efficacy and define the use of adjunctive therapies in conjunction with the CAR T cells. A further goal is to evaluate ROR1 expression and signaling in differing clinical states of malignant prostate cancer and define PCa cell types that express the ROR-1 oncoprotein. An additional goal is to determine efficacy of ROR1 CART cells against patient biopsy-derived organoids in vitro using ROR1 CAR engineered into TILs and PBMCs from the same patient.

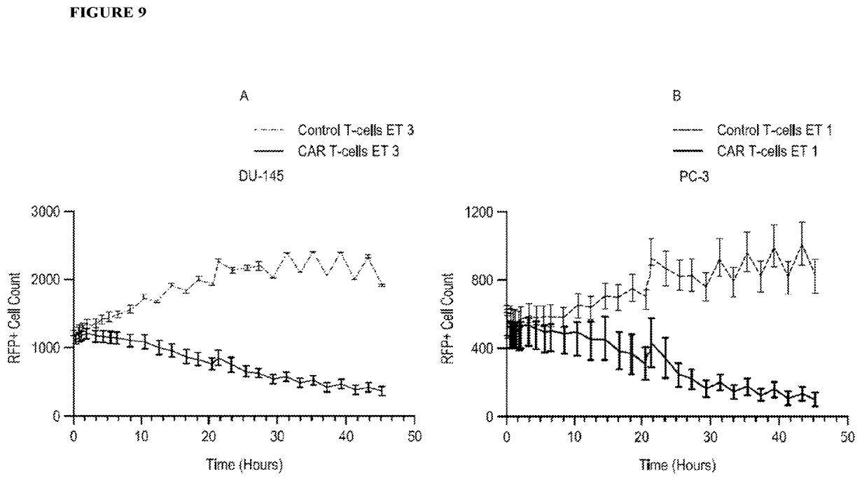

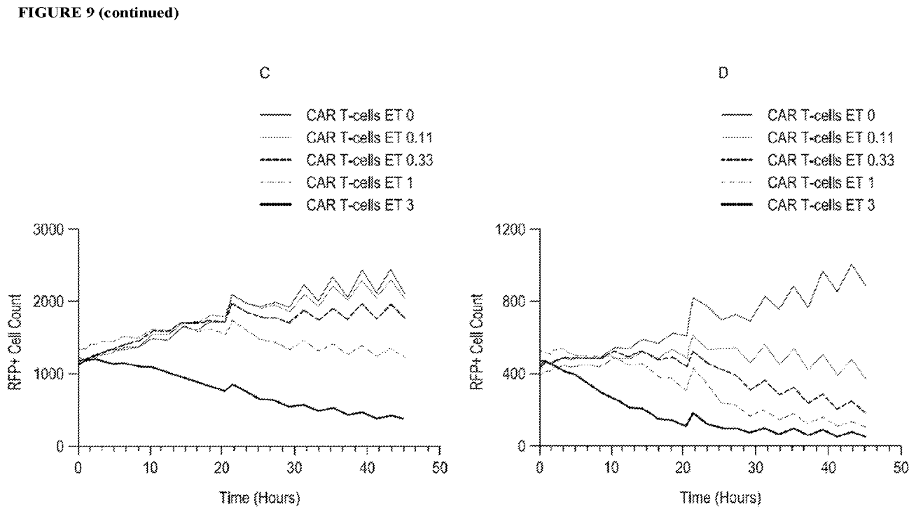

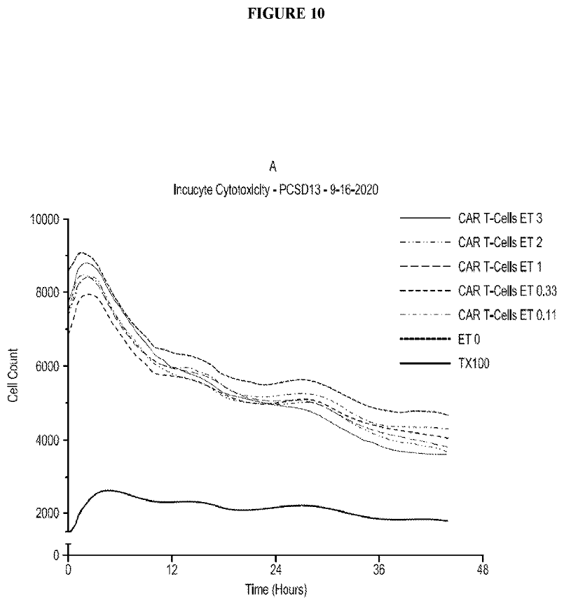

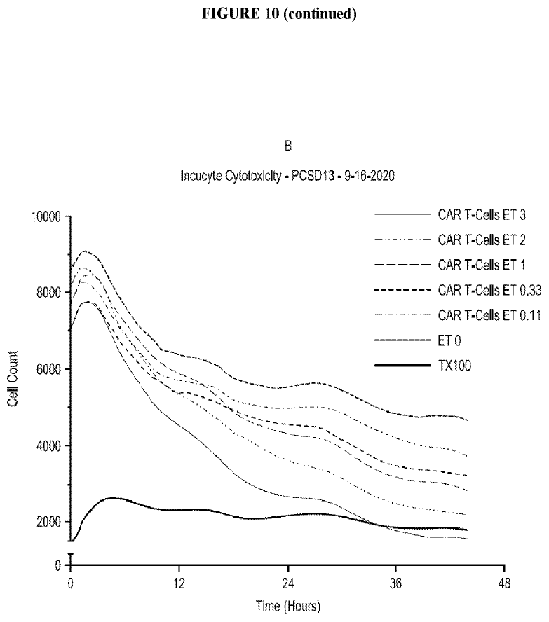

As shown in FIG. 9, the generated anti-ROR1 T-cell CARs are highly potent in targeting and killing ROR1 expressing NEPC cell lines even at low effector to target (E:T) ratios. FIG. 10 shows that anti-ROR1 CAR-T cells specifically killed the ROR1^{pos} PDX cells in Effector:Target (E:T) dose-dependent manner. Thus, the anti-ROR1 T-cell CAR is highly potent in targeting and killing ROR1 expressing NEPC cell lines even at low effector to target (E:T) ratios.

### Pilot Study

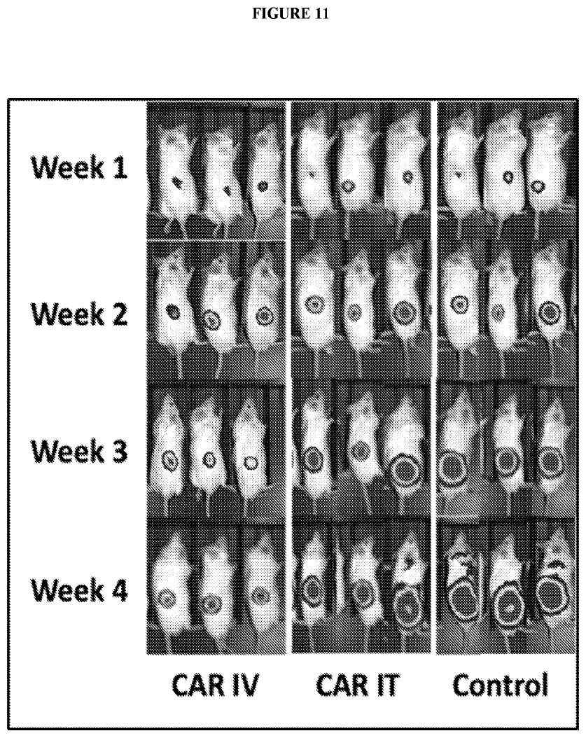

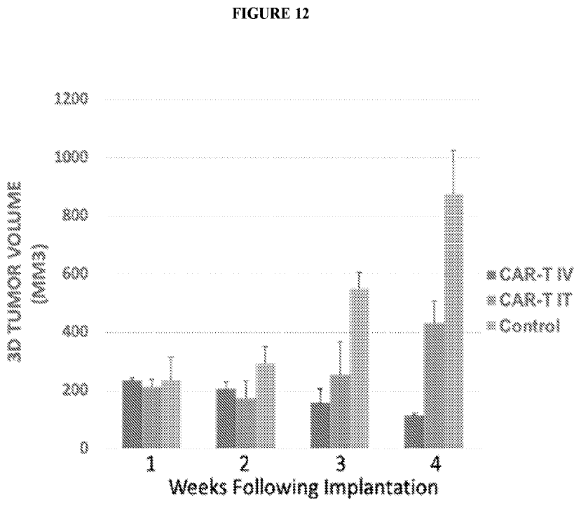

A pilot study was conducted to determine the effect of the CAR T cells provided herein on a prostate cancer cell line implanted subcutaneously. PC3 cells transduced with luciferase were implanted sub-cutaneously into the flanks of immuno-deficient NSG mice. For treatment groups, we administered the ROR1 T-cell CAR both intravenously and by direct injection into the tumor. The control animals received mock transduced activated T-cells generated from the same donor leukapheresed product. As shown in FIG. 11, the PC3 cells rapidly expanded in the backs of these animals and by week 4 had expanded into large bulky tumors and the mice had to be sacrificed. In contrast, the CAR-T treated mice that received a single intravenous or intra-tumoral injection had reduced disease burden when compared to control animals, and had only minimal amounts of disease at the end of the study.

To determine the efficacy of ROR1 CART cells in PCa cell lines and PDX models of mCRPC and NEPC in vivo alone and in combination, with docetaxel, the effect of the anti-ROR-1 CAR T cells on tumor growth inhibition is examined in PCa cell lines and PDX PCSD1, and PCSD13 organoids.

The anti-ROR-1 CAR T cells are tested in PCa Cell lines and PCa organoids in vitro and in sub-cutaneous, intra-femoral bone and intra-cardiac injected PCa cell line xenografts and PDXs in vivo.

The outline of these experiments in pre-clinical models for metastatic prostate cancer is shown below:

Cells: Prostate cancer cell lines: AR+ROR1^{low}: LNCaP; ARV7+: VCaP; AR−ROR1^{high}; PC3; AR−ROR1^{high}: DU145, PCa cells lines all stably express RFP-luciferase and cytotoxicity assay will be performed in Incucyte. Prostate Cancer Bone Metastasis PDX Organoids AR+ROR1^{low}: PCSD1 and AR+ROR^{high} PCSD13.

Therapy: cirmtuzumab-based anti-ROR1 CART cells.

In vitro Experiment 1. Perform titration of Effector:Target (E:T) ratios of CART cell experiments on four PCa cell lines alone or in combination with Docetaxel. Four Effector to Target (E:T) ratios are tested: 30:1, 10:1, 3:1, 1:1 ROR1 CART or control T cells alone+/−anti-androgen+/−docetaxel in triplicate per 96-well plate×2 plates per cell line)×4 prostate cancer cell lines. Cell Viability is measured using Incucyte for RFP-labeled cells in 96-well plate assays. Cytotoxic killing is measured in LDH-release assay.

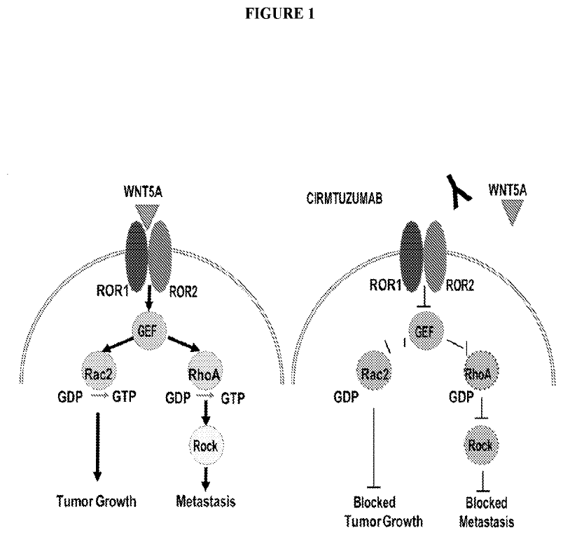

In vitro Experiment 2. Titration of E:T experiments is performed on PCSD1-GFP-luciferase, PCSD13-GFP-luciferase for CART as above in Incucyte assay. Test 3D cultures of 2 PDOs (PCSD1 and PCSD13) in groups: Vehicle control, E:T ratios 30:1. 10:1, 3:1, 1:1 CART or control T cells+/−Enzalutamide+/−Docetaxel in triplicate 24-well plates (see FIG. 1)=2 PDOs×one 24-well plate×2 experiments (duplicates)=4 plates. Weekly digital microscope imaging and analysis of organoid cell number (viability) and size (proliferation). Determine AR protein levels and organoid differentiation using Immunohistochemistry (IHC) of AR and Immunofluorescence (IFC) analysis of prostate markers: Cytokeratin (CK) 5, CK8, p63; Proliferation: Ki67 and Apoptosis: Cleaved Caspase 3. Measure effects on AR expression and AR-dependent gene expression using qRT-PCR and RNAseq. Experiment are performed in triplicate plates. Dose titration treatment starting at Week 1 after doming organoids. Images at pre-treatment vs. 3 h post-treatment (hrs.), 6 h, 12 h, 24 h. Collect cells for RNA, lysate after 24 h for AR degradation analysis by IHC and FFPE sections for IHC and IFC.

In vivo PCa cell line Xenograft and Patient derived xenograft (PDX) Experiments:

Mice: NSG (NOD.Cg-Prkdcscid Il2rgtm1Wjl/SzJ, Jackson Labs Stock #005557), 6-8 weeks old, male.

Cells: LNCaP (AR+, enzalutamide sensitive) a.) sub-cutaneous (s.c.), b.) intra-cardiac (i.e.) injection., 2. PC3 (AR negative, enzalutamide resistant) a.) sub-cutaneous (s.c.), b.) intra-cardiac (i.e.) injection, 3. DU145 (AR negative, enzalutamide resistant) a.) sub-cutaneous (s.c.), b.) intra-cardiac (i.e.) injection, 4. VCaP (AR+, enzalutamide resistant) a.) sub-cutaneous (s.c.), b.) intra-cardiac (i.e.) injection, 5. PCSD1 PDX cells from bone metastasis of prostate adenoma carcinoma (AR+, enzalutamide resistant in the femur bone environment) a.) sub-cutaneous (s.c.), b.) intra-femoral (i.f.) injection, 6. PCSD13 PDX cells bone metastasis of prostate small cell carcinoma (AR+, enzalutamide resistant in the femur bone environment) a.) sub-cutaneous (s.c.), b.) intra-femoral (if.) injection.

Therapy: Cirmtuzumab-based Anti-ROR1 CART cells Treatments start when tumors are at least 100 mm³. For CART treatment group n=10 mice (depending in tumor size at start of treatment): 1. Control T cells (no CAR), Vehicle (n=10), 2. CART cells, Vehicle (n=10), 3. Control T cells, Docetaxel, (n=10), 4. CART cells, Docetaxel, (n=10).

Total n=40 mice×6 Xenograft (LNCaP, PC3, DU145, VCaP, PCSD1, PCSD13) models=240 mice.

For CART compared to control T cells each treatment group n=8-10 mice (depending in tumor size at start of treatment): 1. Control T cells, Vehicle (n=8-10), 2. CART cells, Vehicle (n=8-10), 3. Control T cells, Enzalutamide, (n=8-10),4. CART cells, Enzalutamide, (n=8-10) Total n=40 mice×4 (LNCaP, VCaP, PCSD1, PCSD13) Xenograft mouse models=240 mice.

Monitor clinical signs daily+caliper measurements of tumor volumes, body weights and health report twice weekly. After 4 weeks of treatment the experiments are terminated for collection of a.) peripheral blood: plasma for drug concentration (PK/PD), serum (PSA), b. Tumors from all mice: Fixing (¼ of tumor) in 4% PFA, Flash freezing (¼) in liquid nitrogen for DNA, RNA, protein analysis, Half of tumor dissociated to single cells for organoids, cryopreservation in 10% DMSO, 90% FBS for FACS on tumor cells, future re-transplantation to measure tumor-initiating cancer stem cells. Injected and contra-lateral non-injected Femurs with surrounding tissue will be formalin fixed and de-calcified for IHC and IFC.

Characterization and improved efficacy of ROR1 CART cells in PCa cell line xenograft and PDX models of mCRPC and NEPC alone or subsequent therapy with docetaxel in vivo.

The ability of ROR-1 CAR T cells to eradicate a range of different types of already established PCa tumors is tested in vivo. Human PCa cell lines are implanted sub-cutaneously (s.c.) in immunodeficient mice (NSG). Human patient-derived PCa bone metastasis xenografts (PDX) are directly injected intra-femorally (i.f) into the endosteal bone marrow space of the right femur to test the efficacy of the CARTs against bone metastases or sub-cutaneously (s.c.) for comparison to the PCa cell lines. Tumor growth inhibition by CART cells alone is compared to SOC therapies: docetaxel and the ADT, enzalutamide, alone and in combination.

### In Vivo PCa Cell Line Xenograft and Patient Derived Xenograft (PDX) Experiments Outline:

Mice: NSG (NOD.Cg-Prkdcscid Il2rgtm1Wjl/SzJ, Jackson Labs Stock #005557), 6-8 weeks old, male.

Cells: 1. PC3 (AR^{neg}, ROR1^{pos} enzalutamide resistant) sub-cutaneous (s.c.), 2. DU145 (AR^{neg}, ROR1^{pos}s, enzalutamide-resistant) sub-cutaneous (s.c.), 3. PCSD13 PDX cells bone metastasis of prostate small cell carcinoma (AR^{pos}, ROR1^{pos} enza resistant in the femur bone environment) a.) sub-cutaneous (s.c.), b.) intra-femoral (i.f).

Therapy: Cirmtuzumab Anti-ROR1 CART cells Treatments will start when tumors are at least 100 mm³. 4. LNCaP (AR^{pos}, ROR1^{low} enzalutamide sensitive), sub-cutaneous (s.c

In vivo Expt 1: CART plus docetaxel: For CART treatment group n=10 mice (depending in tumor size at start of treatment): 1. Control T cells (no CAR), Vehicle (n=10), 2. CART cells, Vehicle (n=10), 3. Control T cells, docetaxel, (n=10),4. CART cells, docetaxel, (n=10).

Test ROR1+ cells: Total n=40 mice×4 Xenograft PC3 s.c., DU145 s.c., PCSD13 s.c., i.f. models=160 mice

In vivo Expt 2: CART plus enzalutamide: For CART compared to control T cells each treatment group n=10 mice (depending in tumor size at start of treatment): 1. Control T cells, Vehicle (n=10), 2. CART cells, Vehicle (n=10), 3. Control T cells, enzalutamide, (n=10),4. CART cells, enzalutamide, (n=10).

Test AR+, ROR1+ cells: 1. LNCaP (s.c.), 2. PCSD13 (s.c.) and 3. PCSD13 (i.f),

Total n=40 mice×3 (LNCaP AR^{pos}, ROR1^{low}sc, PCSD13 s.c., PCSD13 i.f) xenograft mouse models=120 mice

### Primary Endpoints:

Tumor growth inhibition: Caliper measurements of tumor volumes, body weights and health report twice weekly plus weekly in vivo bioluminescence (IVIS).

Serum collection weekly: PSA and ALP assays.

Termination Endpoint: After 4 weeks of treatment, peripheral blood, spleen and bone marrow are collected and analyzed for CART presence using FACS, plasma for drug concentration (PK/PD), serum (PSA). Tumors from all mice: Fix (¼ of tumor) in 4% PFA, flash freezing (¼) in liquid nitrogen for DNA, RNA, protein analysis, Half of tumor will be dissociated into single cells for organoids, FACS on tumor cells, and tumor infiltrating CART cells, cryopreservation in 10% DMSO, 90% FBS for future re-transplantation to measure tumor-initiating cancer stem cells. Injected and contra-lateral non-injected Femurs with surrounding tissue are formalin fixed and decalcified for IHC and IFC. Flash frozen samples are used for extraction of DNA, RNA for RNASeq, qPCR of PSA, AR, and protein for immunoblotting analysis of signaling pathways: WNT5A, ROR1, ROR2, Phospho-Tyrosines, AKT and ERK. Femur bone plus periosteal tumor tissue: Formalin fixed for MicroCT, then paraffin-embedded for FFPE sections, immunohistochemistry (PSA, ROR1 and ROR2, other Frizzled proteins) and immunofluorescence (AR, CK5, CK8, Ki67, cleaved caspase3).

In Vivo Testing of Anti-ROR1-CAR T Cells in De Novo Bone Metastasis Models Using Intra-Cardiac or Intra-Iliac Artery Injection Models of Bone Metastasis Alone or in Combination with ADT (Enzalutamide) and/or Docetaxel.

The efficacy of the cirmtuzumab-based CAR T cells is tested against de novo metastasis. alone or subsequent to therapy with docetaxel in treating mice engrafted with ROR1 expressing cell lines, PC3, DU145 and PDX PCSD13 via intra-cardiac and intra-iliac xenograft artery injections. Mice are first treated with docetaxel or enzalutamide for at least 28 days to de-bulk tumors. Following chemotherapy treatment and washout, the PCa is treated with the CART cells.

### Primary Endpoints:

Tumor growth inhibition: weekly IVIS body weight, weekly IVIS, weekly serum collection, and weekly PSA and ALP assays.

Termination Endpoint: Collect tumor tissue to be flash frozen for subsequent extraction of DNA, RNA for RNASeq, qPCR of PSA, AR, and ROR1 and protein for immunoblotting analysis of signaling pathways: WNT5A, ROR1, ROR2, Phospho-Tyrosines, AKT, ERK.

Outcome: cirmtuzumab-based CAR T cells inhibit tumor cell growth of ROR1+ metastatic prostate cancer lines and PDX in vitro and in vivo alone or in combination with prior SOCs docetaxel or enzalutamide.

Development of a Companion Diagnostic to Detect ROR1 Expressing CSCs Inpatients that have CRPC.

The aim of this study is to develop a CLIA-certified IHC assay that can be employed to detect ROR-1 in neuroendocrine cancers, such as CPRC.

The anti-ROR1 antibody employed in the development of this assay is mAb (4A5). In this process 4A5 is tested using Tissue Microarrays (TMAs) of normal, inflammatory, and neoplastic (benign and malignant) tissues provided by the Biorepository Tissue Technology Shared Resource (BTTSR) facility at UC San Diego. Both frozen and paraffin embedded TMAs from the relevant pathologies are used, including patient-derived prostate tumor samples collected at various pathological states and matching normal tissue controls. For validation purposes, the antibody is tested using two standard IHC techniques, the standard and highly sensitive ABC technique (Vector, CA), as described, and the ImmPRESS™ HRP Polymer Detection Kit, as well as standard indirect immune-fluorescence.

TMA selection: The prostate tissue TMAs is constructed as described, using the MicaArray GEN 3.0 manual arrayer (Micatu Inc, New York). A cohort of de-identified paraffin blocks is selected from the BTTSR, representing a range of non-malignant and malignant prostate tumors. Negative tissue controls are arrayed together with known positives samples previously detected on ROR1^{pos} cancers of the ovary, colon, lung, skin, and pancreas.

Analysis of Protein Expression: The Study Pathologist and a pathologist blinded to the results or the techniques evaluate all TMAs and individual tissues before IHC analysis using H&E-stained slides, for relevancy and tissue preservation. The number of positive cells is visually evaluated for each core and the results are expressed as a percentage of stained cells/total number of cells. Based on their immunoreactivity, the TMA cores are divided and scored in five categories: 0, less the 10% of stained cells; 1, between 10% and 25%; 2, between 25% and 50%; 3, 50% to 75%; and 4, 75% to 100% of cells stained. Quantitative automated image analysis are explored.

Statistical analysis: Pearson's correlation coefficient is used to quantify agreement between measurements, as described by Anagnostou et al. Cohen's weighted kappa is also used to measure agreement between antibody pairs.

CLIA Assay Validation-including: specificity, sensitivity, reproducibility and limits. A set of samples from the CRPC TMAs is analyzed by a study pathologist on consecutive days to determine the day-to-day variability, reproducibility and robustness of assay performance. In a second set of studies, two independent pathologists read 40 specimens from ROR1^{pos} and ^{neg} samples. Inter-rater agreement are quantified by Pearson's correlation and Cohen's weighted kappa, together with their 95% confidence limits. Based on the results of these studies, a CLIA validated IHC companion assay is developed to allow enrollment of patients with ROR1^{pos} CRPC into clinical studies with the cirmtuzumab-based T-cell CAR.

### Biosystem Cultures

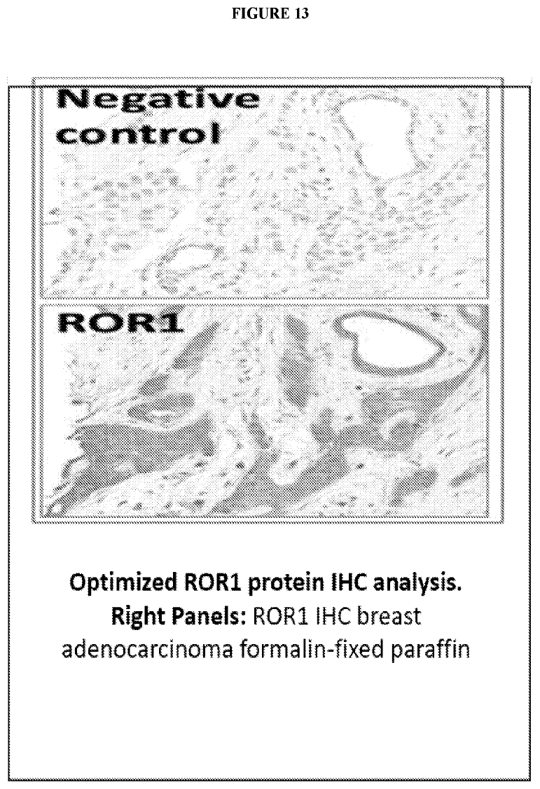

The aim of this study was to determine the efficacy of ROR1 CART cells against patient biopsy-derived organoids in vitro using ROR1 CAR engineered into TILs and PBMCs from the same patient. Personalized mini-tumors in cell cultures have been successfully generated from tumor tissues of prostate cancer (PCa) patients (Lee et al, 2020). Samples of each patient's tumor tissues were collected immediately after surgical removal and used to establish 3D-organoids, or mini-tumors. In parallel, we expanded each patient's own immune cells that were residing within the tumor tissue known as tumor infiltrating lymphocytes (TILs). We then combined each patient's mini-tumors with their own TILs to establish personalized 3D-Biosystem cultures as shown in FIG. 13. These personal patient-derived mini-tumors are being used to determine responses to immunotherapies in combination with current and novel tumor targeting therapies.

Organoids are established and TILs are expanded from n=5-10 patient biopsies from prostate cancer metastases. Cirmtuzumab-based CART cells are generated from TILs and PBMCs from the same patients as the organoids using our lentiviral construct that we used in our CART product. Organoids and patient-derived cirmtuzumab-based CARTs are co-cultured. Cytotoxic killing is measured in digital microscope daily imaging at time points: day1-7. Single cell RNA sequencing (UCSD IGM core, 10× Genomics) and immunofluorescence cytochemistry of co-cultures with immune cell and prostate markers.

The results of this study allow for the development of a strategy to tailor immune therapy for individual patients through a better understanding of the mechanisms of response and resistance.

Evaluation of ROR1 Expression and Signaling in Differing Clinical States of Malignant Prostate Cancer and Definition of PCa Cell Types that Express ROR-1.

ROR1 expression patterns and signaling are studied in samples derived from patients with localized prostate cancer of differing Gleason score, metastatic hormone sensitive prostate cancer, metastatic CRPC, and neuroendocrine prostate cancer to determine whether ROR1 expression is increased in higher grade and castrate resistant tumors.

FFPE tissue from standard of care radical prostatectomy, prostate biopsy, and metastasis biopsy archived in the UCSD Pathology Biorepository are analyzed. IHC staining is performed with optimal commercially available anti-ROR1 antibody, such as 4A5 (BD) or Protein Tech, a rabbit polyclonal in the UCSD Tissue Technology Shared Resource histology core. IHC-stained slides are reviewed, and the areas of tumor are manually identified and marked. All slides are scanned in the Tissue Technology Shared Resource using the Aperio ScanScope XT system (Aperio®; Vista, CA). The Spectrum Analysis algorithm package and ImageScope analysis software are applied to quantify IHC staining using a color deconvolution algorithm as previously described. (60,61).

Clinical specimens are also reviewed to determine Gleason score and presence of neuroendocrine differentiation. Clinical parameters including age, race, ethnicity, PSA, stage, and prior treatment status are captured and linked to pathologic and ROR1 expression data. The expression of ROR1 is assessed and graded as follows: 0: No positive cancer cells, 1: Low-moderate staining to <50% of cancer cells, 2: Intense staining of <50% of cancer cells or moderate staining to >50% of cancer cells; 3: Intense staining on >50% of cancer cells. All staining is evaluated and ROR1 levels are correlated with disease parameters including imaging response of measurable disease, PSA response (PSA decline ≥50% from baseline), or stable disease ≥6 months. In addition, the effect of cirmtuzumab-based CART inhibition of ROR1 signaling is evaluated using antibodies to phospho-SRC, phospho-AKT, phospho-CREB, YAP/TAZ, and BMI1 in serial sections of the biopsies.

Statistical analysis: Table 1 details the number of archived specimens available for use. For the primary analysis, we investigate comparisons in ROR1 expression and downstream signaling between castration resistant disease (n=20) and hormone sensitive disease (n=70). We define positive ROR1 expression as an TIC grading of 1 to 3. We compute the proportion of ROR1 positive specimens for each clinical state and its 95% CIs. Fisher's exact test is used to compare the proportion of ROR1 positivity between castrate resistant and hormone sensitive clinical states.

The results indicate that ROR1 expression rate is 20% in hormone sensitive specimens and 60% in castrate resistant specimens with a power of 90% and a two-sided alpha of 0.05, with our current sample size.

TABLE 1
Archived specimens to be procured from UCSD Pathology Biorepository

| | Number of Specimens |
|---|---|
| Clinical State | Available for Use |
| Radical Prostatectomy - Gleason 6 | 10 |
| Radical Prostatectomy - Gleason 7 | 20 |
| Radical Prostatectomy - Gleason 8-10 | 30 |
| Metastasis Biopsy - Hormone sensitive | 10 |
| Metastasis Biopsy - Castration resistant | 20 |
| Neuroendocrine | 10 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

### Embodiments

Embodiment 1. A chimeric antigen receptor (CAR) comprising:
i. an antibody binding region, wherein the antibody binding region specifically binds ROR-1 and wherein the antibody binding region comprises a light chain variable domain and a heavy chain variable domain;
   wherein the light chain variable domain comprises a CDR L1 as set forth in SEQ ID NO: 43, a CDR L2 as set forth in SEQ ID NO: 44 and a CDR L3 as set forth in SEQ ID NO: 45; and the heavy chain variable domain comprises a CDR H1 as set forth in SEQ ID NO: 46, a CDR H2 as set forth in SEQ ID NO: 47, and a CDR H3 as set forth in SEQ ID NO: 48;
ii. a spacer domain;
iii. a transmembrane domain; and iv. an intracellular domain.

Embodiment 2. A chimeric antigen receptor (CAR) comprising:
v. an antibody binding region, wherein the antibody binding region specifically binds ROR-1 and wherein the antibody binding region comprises a light chain variable domain and a heavy chain variable domain;
   wherein the light chain variable domain comprises a CDR L1 as set forth in SEQ ID NO: 49, a CDR L2 as set forth in SEQ ID NO: 50 and a CDR L3 as set forth in SEQ ID NO: 51; and the heavy chain variable domain comprises a CDR H1 as set forth in SEQ ID NO: 52, a CDR H2 as set forth in SEQ ID NO: 53, and a CDR H3 as set forth in SEQ ID NO: 54;
vi. a spacer domain;
vii. a transmembrane domain; and
viii. an intracellular domain.

Embodiment 3. The chimeric antigen receptor of any one of embodiments 1-2, wherein the spacer domain is between 14 and 120 amino acids in length.

Embodiment 4. The chimeric antigen receptor of any one of embodiments 1-3, wherein the light chain variable domain is linked to the N-terminus or the C-terminus of the heavy chain variable domain.

Embodiment 5. The chimeric antigen receptor of any one of embodiments 1-4, wherein the light chain variable domain is covalently linked to the heavy chain variable domain through a polypeptide linker.

Embodiment 6. The chimeric antigen receptor of embodiment 5, wherein the polypeptide linker comprises an amino acid sequence of SEQ ID NO: 24.

Embodiment 7. The chimeric antigen receptor of any one of embodiments 1-6, wherein the spacer domain comprises an antibody domain.

Embodiment 8. The chimeric antigen receptor of embodiment 7, wherein the antibody domain comprises an immunoglobulin hinge domain, an immunoglobulin constant heavy chain 3 (CH3) domain, an immunoglobulin constant heavy chain 2 (CH2) domain, or a combination thereof.

Embodiment 9. The chimeric antigen receptor of any one of embodiments 1-8, wherein the spacer domain comprises an amino acid sequence of SEQ ID NO: 29, SEQ ID NO: 41 or SEQ ID NO: 42.

Embodiment 10. The chimeric antigen receptor of any one of embodiments 1-9, wherein the light chain variable domain comprises an amino acid sequence at least about 90%, 95%, 97%, 98%, 99%, or 100% identical to SEQ ID NO: 21.

Embodiment 11. The chimeric antigen receptor of any one of embodiments 1-10, wherein the heavy chain variable domain comprises an amino acid sequence at least about 90%, 95%, 97%, 98%, 99%, or 100% identical to SEQ ID NO: 27.

Embodiment 12. The chimeric antigen receptor of any one of embodiments 1-11, wherein the transmembrane domain comprises a CD8α transmembrane domain, a CD28 transmembrane domain, a CD4 transmembrane domain, a CD3ζ transmembrane domain, or any combination thereof.

Embodiment 13. The chimeric antigen receptor of embodiment 12, wherein the transmembrane domain is a CD28 transmembrane domain.

Embodiment 14. The chimeric antigen receptor of any one of embodiments 1-13, wherein the intracellular domain comprises an intracellular co-stimulatory signaling domain, an intracellular T-cell signaling domain, or a combination thereof.

Embodiment 15. The chimeric antigen receptor of embodiment 14, wherein the intracellular co-stimulatory signaling domain is a 4-1BB intracellular co-stimulatory signaling domain, a CD28 intracellular co-stimulatory signaling domain, a ICOS intracellular co-stimulatory signaling domain, an OX-40 intracellular co-stimulatory signaling domain, or any combination thereof.

Embodiment 16. The chimeric antigen receptor of embodiment 15, wherein the intracellular costimulatory signaling domain comprises a 41-BB intracellular co-stimulatory signaling domain.

Embodiment 17. The chimeric antigen receptor of embodiment 16, wherein the intracellular costimulatory signaling domain comprises a CD28 intracellular co-stimulatory signaling domain and a 41-BB intracellular co-stimulatory signaling domain.

Embodiment 18. The chimeric antigen receptor of any one of embodiments 15-17, wherein the intracellular costimulatory signaling domain further comprises an intracellular T-cell signaling domain.

Embodiment 19. The chimeric antigen receptor of embodiment 18, wherein the intracellular T-cell signaling domain is a CD3ζ intracellular T-cell signaling domain.

Embodiment 20. The chimeric antigen receptor of any one of embodiments 1-19, wherein the chimeric antigen receptor binds to a cell.

Embodiment 21. The chimeric antigen receptor of embodiment 20, wherein the cell is a neuroendocrine cancer cell.

Embodiment 22. The chimeric antigen receptor of embodiment 21, wherein the neuroendocrine cancer cell is a carcinoid tumor cell, an islet cell tumor cell, a medullary thyroid cancer cell, a pheochromocytoma cell, a neuroendocrine carcinoma of the skin cell, small cell lung cancer cell, large cell neuroendocrine carcinoma cell, neuroendocrine prostate cancer (NEPC) cell, or metastatic castration-resistant prostate cancer (CRPC) cell.

Embodiment 23. The chimeric antigen receptor of any one of embodiments 1-22, wherein the chimeric antigen receptor forms part of a cell.

Embodiment 24. The chimeric antigen receptor of any one of embodiments 1-23, wherein the chimeric antigen receptor forms part of a T cell.

Embodiment 25. An isolated nucleic acid encoding a chimeric antigen receptor of any one of embodiments 1-24.

Embodiment 26. A pharmaceutical composition comprising a therapeutically effective amount of a chimeric antigen receptor of any one of embodiments 1-25, and a pharmaceutically acceptable excipient.

Embodiment 27. A method of treating a neuroendocrine cancer in a subject in need thereof, wherein the method comprises administering to the subject an effective amount of the pharmaceutical composition of embodiment 26.

Embodiment 28. The method of embodiment 27, wherein the chimeric antigen receptor (CAR) binds to a neuroendocrine cancerous cell.

Embodiment 29. The method of any one of embodiments 27-28, wherein the cell is a T lymphocyte.

Embodiment 30. The method of embodiment 29, wherein the T lymphocyte is a CD4+T lymphocyte or a CD8+T lymphocyte.

Embodiment 31. The method of any one of embodiments 27-30, wherein the cell is a natural killer cell.

Embodiment 32. The method of embodiment 31, wherein the natural killer cell is autologous to the subject.

Embodiment 33. The method of embodiment 31, wherein the natural killer cell is heterologous to the subject.

Embodiment 34. The method of embodiment 31, wherein the natural killer cell is allogenic to the subject.

Embodiment 35. The method of any one of embodiments 27-34, wherein the neuroendocrine cancer is a carcinoid tumor, an islet cell tumor, a medullary thyroid cancer, a pheochromocytoma, a neuroendocrine carcinoma of the skin, small cell lung cancer, large cell neuroendocrine carcinoma, neuroendocrine prostate cancer (NEPC), or metastatic castration-resistant prostate cancer (CRPC).

Embodiment 36. The method of any one of embodiments 27-35, wherein the neuroendocrine cancer is metastatic castration-resistant prostate cancer (CRPC).

Embodiment 37. The method of any one of embodiments 27-36, wherein neuroendocrine cancerous cells express ROR-1.

Embodiment 38. The method of any one of embodiments 27-37, wherein the subject has failed to respond to androgen deprivation therapy.

Embodiment 39. The method of any one of embodiments 27-38, wherein the neuroendocrine cancer has metastasized to bone.

Embodiment 40. The method of any one of embodiments 27-39, wherein the method further comprises administering cirmtuzumab to the subject.

Embodiment 41. The method of embodiment 40, wherein the cirmtuzumab and the cell are administered separately.

Embodiment 42. The method of embodiment 40, wherein the cirmtuzumab and the cell are administered together.

Embodiment 43. The method of any one of embodiments 27-42, wherein the method further comprises administering to the subject platinum-based chemotherapy.

Embodiment 44. The method of embodiment 43, wherein the platinum-based chemotherapy comprises carboplatin, cisplatin, etoposide, a taxane, or any combination thereof.

Embodiment 45. A method of preventing or treating metastasis in a subject with a neuroendocrine cancer, wherein the method comprises administering to the subject an effective amount of the pharmaceutical composition of embodiment 26.

Embodiment 46. The method of embodiment 45, wherein the chimeric antigen receptor (CAR) binds to a neuroendocrine cancerous cell.

Embodiment 47. The method of any one of embodiments 45-46, wherein the cell is a T lymphocyte.

Embodiment 48. The method of embodiment 47, wherein the T lymphocyte is a CD4+T lymphocyte or a CD8+T lymphocyte.

Embodiment 49. The method of any one of embodiments 45-48, wherein the cell is a natural killer cell.

Embodiment 50. The method of embodiment 49, wherein the natural killer cell is autologous to the subject.

Embodiment 51. The method of embodiment 49, wherein the natural killer cell is heterologous to the subject.

Embodiment 52. The method of embodiment 49, wherein the natural killer cell is allogenic to the subject.

Embodiment 53. The method of any one of embodiments 45-52, wherein the neuroendocrine cancer is a carcinoid tumor, an islet cell tumor, a medullary thyroid cancer, a pheochromocytoma, a neuroendocrine carcinoma of the skin, small cell lung cancer, large cell neuroendocrine carcinoma, neuroendocrine prostate cancer (NEPC), or metastatic castration-resistant prostate cancer (CRPC).

Embodiment 54. The method of any one of embodiments 45-53, wherein the neuroendocrine cancer is metastatic castration-resistant prostate cancer (CRPC).

Embodiment 55. The method of any one of embodiments 45-54, wherein neuroendocrine cancerous cells express ROR-1.

Embodiment 56. The method of any one of embodiments 45-55, wherein the subject has failed to respond to androgen deprivation therapy.

Embodiment 57. The method of any one of embodiments 45-56, wherein the neuroendocrine cancer has metastasized to bone.

Embodiment 58. The method of any one of embodiments 45-57, wherein the method further comprises administering cirmtuzumab to the subject.

Embodiment 59. The method of embodiment 58, wherein the cirmtuzumab and the cell are administered separately.

Embodiment 60. The method of embodiment 58, wherein the cirmtuzumab and the cell are administered together.

Embodiment 61. The method of any one of embodiments 45-60, wherein the method further comprises administering to the subject platinum-based chemotherapy.

Embodiment 62. The method of embodiment 61, wherein the platinum-based chemotherapy comprises carboplatin, cisplatin, etoposide, a taxane, or any combination thereof.

Embodiment 63. The method of any one of embodiments 43 and 61, wherein the platinum-based chemotherapy comprises administering an antibody-drug conjugate.

### REFERENCES

1. Siegel R, Ma J, Zou Z, & Jemal A. Cancer statistics. CA Cancer J Clin 2014: 64(1):9-29.
2. Oster G, Lamerato L, Glass A G, Richert-Boe K E, Lopez A, Chung K, Richhariya A, Dodge T, Wolff G G, Balakumaran A, Edelsberg J. Natural history of skeletal-related events in patients with breast, lung, or prostate cancer and metastases to bone: a 15-year study in two large US health systems. Support Care in Cancer. 2013 Dec. 21(12):3279-3286.
3. Logothetis C, Morris M J, Den R, Coleman R E. Current perspectives on bone metastases in castrate-resistant prostate cancer. Cancer Metastasis Rev. 2018 March; 37(1):189-196. doi: 10.1007/s10555-017-9719-4. Review. PMID:29380085
4. Garnero P, Buchs N, Zekri J, Rizzoli R, Coleman R E, Delmas P D. Markers of bone turnover for the management of patients with bone metastases from prostate cancer. Br J Cancer. 2000 82(4):858-864.
5. Shiao S L, Chu G C, Chung L W. Regulation of prostate cancer progression by the tumor microenvironment. Cancer Lett. 2016 Jan. 28. pii: S0304-3835(15)00773-9. doi: 10.1016/j.canlet.2015.12.022. [Epub ahead of print].
6. Vargas H A, Wassberg C, Fox J J, Wibmer A, Goldman D A, Kuk D, Gonen M, Larson S M, Morris M J, Scher H I, Hricak H. Bone metastases in castration-resistant prostate cancer: associations between morphologic CT patterns, glycolytic activity, and androgen receptor expression on PET and overall survival. Radiology. 2014 April; 271(1):220-9. doi: 10.1148/radiol.13130625. Epub 2013 Nov. 18. PMID:24475817
7. Amend S R1, Roy S2, Brown JS3, Pienta KJ2. Ecological paradigms to understand the dynamics of metastasis. Cancer Lett. 2016 Sep. 28; 380(1):237-42. doi: 10.1016/j.canlet.2015.10.005. Epub 2015 Oct. 10.
8. Shiozawa Y1,2, Berry JE1, Eber MR1,2, Jung Y1, Yumoto K1, Cackowski FC1, Yoon HJ3, Parsana P4,5, Mehra R6, Wang J1, McGee S1, Lee E1, Nagrath S3, Pienta KJ4, Taichman RS1. The marrow niche controls the cancer stem cell phenotype of disseminated prostate cancer. Oncotarget. 2016 Jul. 5; 7(27):41217-41232. doi: 10.18632/oncotarget.9251.
9. Nguyen H M, Vessella R L, Morrissey C, Brown L G, Coleman I M, Higano C S, Mostaghel E A, Zhang X, True L D, Lam H M, Roudier M, Lange P H, Nelson P S, Corey E. LuCaP Prostate Cancer Patient-Derived Xenografts Reflect the Molecular Heterogeneity of Advanced Disease an--d Serve as Models for Evaluating Cancer Therapeutics. Prostate. 2017 May; 77(6):654-671. doi: 10.1002/pros.23313. Epub 2017 Feb. 3. PMID:28156002
10. Kleb B, Estécio MR, Zhang J, Tzelepi V, Chung W, Jelinek J, Navone N M, Tahir S, Marquez V E, Issa J P, Maity S, Aparicio A. Differentially methylated genes and androgen receptor re-expression in small cell prostate carcinomas. Epigenetics. 2016 Mar. 3; 11(3):184-93. doi: 10.1080/15592294.2016.1146851. Epub 2016 Feb. 18. PMID:26890396
11. Hao J, Ci X, Xue H, Wu R, Dong X, Choi S YC, He H, Wang Y, Zhang F, Qu S, Zhang F, Haegert A M, Gout P W, Zoubeidi A, Collins C, Gleave M E, Lin D, Wang Y. Patient-derived Hormone-naive Prostate Cancer Xenograft Models Reveal Growth Factor Receptor Bound Protein 10 as an Androgen Receptor-repressed Gene Driving the Development of Castration-resistant ProstateCancer.Eur Urol. 2018 Mar. 12. pii: S0302-2838(18)30162-3. doi: 10.1016/j.eururo.2018.02.019. [Epub ahead of print] PMID:29544736
12. Robinson D, Van Allen E M, Wu Y M, Schultz N, Lonigro R J, Mosquera J M, Montgomery B, Taplin M E, Pritchard C C, Attard G, Beltran H, Abida W, Bradley R K, Vinson J, Cao X, Vats P, Kunju L P, Hussain M, Feng F Y, Tomlins S A, Cooney K A, Smith D C, Brennan C, Siddiqui J, Mehra R, Chen Y, Rathkopf D E, Morris M J, Solomon S B, Durack J C, Reuter V E, Gopalan A, Gao J, Loda M, Lis R T, Bowden M, Balk S P, Gaviola G, Sougnez C, Gupta M, Yu E Y, Mostaghel E A, Cheng H H, Mulcahy H, True L D, Plymate S R, Dvinge H, Ferraldeschi R, Flohr P, Miranda S, Zafeiriou Z, Tunariu N, Mateo J, Perez-Lopez R, Demichelis F, Robinson B D, Sboner A, Schiffman M, Nanus D M, Tagawa S T, Sigaras A, Eng K W, Elemento O, Sboner A, Heath E I, Scher H I, Pienta K J, Kantoff P, de Bono J S, Rubin M A, Nelson P S, Garraway L A, Sawyers C L, Chinnaiyan A M. Integrative Clinical Genomics of Advanced Prostate Cancer. Cell. 2015 May 21; 161(5):1215-1228. doi: 10.1016/j.cell.2015.05.001. Erratum in: Cell. 2015 Jul. 16; 162(2):454.
13. Beltran H, Wyatt A W, Chedgy E C, Donoghue A, Annala M, Warner E W, Beja K, Sigouros M, Mo F, Fazli L, Collins C C, Eastham J, Morris M, Taplin M E, Sboner A, Halabi S, Gleave M E. Impact of Therapy on Genomics and Transcriptomics in High-Risk Prostate Cancer Treated with Neoadjuvant Docetaxel and Androgen Deprivation Therapy. Clin Cancer Res. 2017 Nov. 15; 23(22):6802-6811. doi: 10.1158/1078-0432.CCR-17-1034. Epub 2017 Aug. 25. PMID: 28842510
14. Pritchard C C, Mateo J, Walsh M F, De Sarkar N, Abida W, Beltran H, Garofalo A, Gulati R, Carreira S, Eeles R, Elemento O, Rubin M A, Robinson D, Lonigro R, Hussain M, Chinnaiyan A, Vinson J, Filipenko J, Garraway L, Taplin M E, AlDubayan S, Han G C, Beightol M, Morrissey C, Nghiem B, Cheng H H, Montgomery B, Walsh T, Casadei S, Berger M, Zhang L, Zehir A, Vijai J, Scher H I, Sawyers C, Schultz N, Kantoff P W, Solit D, Robson M, Van Allen E M, Offit K, de Bono J, Nelson P S. Inherited DNA-Repair Gene Mutations in Men with Metastatic Prostate Cancer. N Engl J Med. 2016 Aug. 4; 375(5):443-53. doi: 10.1056/NEJMoa1603144. Epub 2016 Jul. 6. PMID:27433846
15. Luo J, Attard G, Balk S P, Bevan C, Burnstein K, Cato L, Cherkasov A, De Bono J S, Dong Y, Gao A C, Gleave M, Heemers H, Kanayama M, Kittler R, Lang J M, Lee R J, Logothetis C J, Matusik R, Plymate S, Sawyers C L, Selth L A, Soule H, Tilley W, Weigel N L, Zoubeidi A, Dehm S M, Raj G V. Role of Androgen Receptor Variants in Prostate Cancer: Report from the 2017 Mission Androgen Receptor Variants Meeting.Eur Urol. 2018 May; 73(5):715-723. doi: 10.1016/j.eururo.2017.11.038. Epub 2017 Dec. 16. PMID:29258679
16. Armenia J, Wankowicz S A M, Liu D, Gao J, Kundra R, Reznik E, Chatila W K, Chakravarty D, Han G C, Coleman I, Montgomery B, Pritchard C, Morrissey C, Barbieri C E, Beltran H, Sboner A, Zafeiriou Z, Miranda S, Bielski C M, Penson A V, Tolonen C, Huang F W, Robinson D, Wu Y M, Lonigro R, Garraway L A, Demichelis F, Kantoff P W, Taplin M E, Abida W, Taylor B S, Scher H I, Nelson P S, de Bono J S, Rubin M A, Sawyers C L, Chinnaiyan A M; PCF/SU2C International Prostate Cancer Dream Team, Schultz N, Van Allen E M; PCF/SU2C International Prostate Cancer Dream Team. The long tail of oncogenic drivers in prostate cancer. Nat Genet. 2018 Apr. 2. doi: 10.1038/s41588-018-0078-z. [Epub ahead of print] PMID:29610475
17. Carlsson A, Kuhn P, Luttgen M S, Dizon K K, Troncoso P, Corn P G, Kolatkar A, Hicks J B, Logothetis C J, Zurita A J. Paired High-Content Analysis of Prostate Cancer Cells in Bone Marrow and Blood Characterizes Increased Androgen Receptor Expression in Tumor Cell Clusters. Clin Cancer Res. 2017 Apr. 1; 23(7):1722-1732. doi: 10.1158/1078-0432.CCR-16-1355. Epub 2016 Oct. 4. PMID:27702818
18. Don-Doncow N1, Marginean F2, Coleman 13, Nelson PS3, Ehrnström R4, Krzyzanowska A1, Morrissey C5, Hellsten R1, Bjartell A6. Expression of STAT3 in Prostate Cancer Metastases. Eur Urol. 2017 March; 71(3):313-316. doi: 10.1016/j.eururo.2016.06.018. Epub 2016 Jun. 22.
19. Wu Y, Schoenborn J R, Morrissey C, Xia J, Larson S, Brown L G, Qu X, Lange P H, Nelson P S, Vessella R L, Fang M. High-Resolution Genomic Profiling of Disseminated Tumor Cells in Prostate Cancer.J Mol Diagn. 2016 January; 18(1):131-43. doi: 10.1016/j.jmoldx.2015.08.004. Epub 2015 Nov. 20.PMID:26607774
20. Haider M, Zhang X, Coleman I, Ericson N, True L D, Lam H M, Brown L G, Ketchanji M, Nghiem B, Lakely B, Coleman R, Montgomery B, Lange P H, Roudier M, Higano C S, Bielas J H, Nelson P S, Vessella R L, Morrissey C. Epithelial mesenchymal-like transition occurs in a subset of cells in castration resistant prostate cancer bone metastases. Clin Exp Metastasis. 2016 March; 33(3):239-48. doi: 10.1007/s10585-015-9773-7. Epub 2015 Dec. 14.
21. Bluemn E G, Coleman I M, Lucas J M, Coleman R T, Hernandez-Lopez S, Tharakan R, Bianchi-Frias D, Dumpit R F, Kaipainen A, Corella A N, Yang Y C, Nyquist M D, Mostaghel E, Hsieh A C, Zhang X, Corey E, Brown L G, Nguyen H M, Pienta K, Ittmann M, Schweizer M, True L D, Wise D, Rennie P S, Vessella R L, Morrissey C, Nelson P S. Androgen Receptor Pathway-Independent Prostate Cancer Is Sustained through FGF Signaling. Cancer Cell. 2017 Oct. 9; 32(4):474-489.e6. doi: 10.1016/j.ccell.2017.09.003. PMID:29017058
22. Cho W J, Oliveira D S, Najy A J, Mainetti L E, Aoun H D, Cher M L, Heath E, Kim H R, Bonfil R D. Gene expression analysis of bone metastasis and circulating tumor cells from metastatic castrate-resistant prostate cancer patients. J Transl Med. 2016 Mar. 15; 14:72. doi: 10.1186/s12967-016-0829-5. PMID:26975354.
23. Davies A H, Beltran H, Zoubeidi A. Cellular plasticity and the neuroendocrine phenotype in prostate cancer. Nat Rev Urol. 2018 Feb. 20. doi: 10.1038/nrurol.2018.22. [Epub ahead of print] Review.PMID: 29460922
24. Bishop J L, Thaper D, Vahid S, Davies A, Ketola K, Kuruma H, Jama R, Nip K M, Angeles A, Johnson F, Wyatt A W, Fazli L, Gleave M E, Lin D, Rubin M A, Collins C C, Wang Y, Beltran H, Zoubeidi A. The Master Neural Transcription Factor BRN2 Is an Androgen Receptor-Suppressed Driver of Neuroendocrine Differentiation in Prostate Cancer. Cancer Discov. 2017 January; 7(1):54-71. doi: 10.1158/2159-8290.CD-15-1263. Epub 2016 Oct. 26. PMID:2778470
25. Dardenne E, Beltran H, Benelli M, Gayvert K, Berger A, Puca L, Cyrta J, Sboner A, Noorzad Z, MacDonald T, Cheung C, Yuen K S, Gao D, Chen Y, Eilers M, Mosquera J M, Robinson B D, Elemento O, Rubin M A, Demichelis F, Rickman D S. N-Myc Induces an EZH2-Mediated Transcriptional Program Driving Neuroendocrine Prostate Cancer. Cancer Cell. 2016 Oct. 10; 30(4):563-577. doi: 10.1016/j.ccell.2016.09.005.PMID: 27728805
26. Zhang W, Liu B, Wu W, Li L, Broom B M, Basourakos S P, Korentzelos D, Luan Y, Wang J, Yang G, Park S, Azad A K, Cao X, Kim J, Corn P G, Logothetis C J, Aparicio A M, Chinnaiyan A M, Navone N, Troncoso P, Thompson T C. Targeting the MYCN-PARP-DNA Damage Response Pathway in Neuroendocrine Prostate Cancer. Clin Cancer Res. 2018 Feb. 1; 24(3):696-707. doi: 10.1158/1078-0432.CCR-17-1872. Epub 2017 Nov. 14. PMID:2913834
27. Lee J K, Phillips J W, Smith B A, Park J W, Stoyanova T, McCaffrey E F, Baertsch R, Sokolov A, Meyerowitz J G, Mathis C, Cheng D, Stuart J M, Shokat K M, Gustafson W C, Huang J, Witte O N. N-Myc Drives Neuroendocrine Prostate Cancer Initiated from Human Prostate Epithelial Cells. Cancer Cell. 2016 Apr. 11; 29(4):536-547. doi: 10.1016/j.ccell.2016.03.001. Epub 2016 Mar. 31.PMID:27050099
28. Singhal U, Wang Y, Henderson J, Niknafs Y S, Qiao Y, Gursky A, Zaslavsky A, Chung J S, Smith D C, Karnes R J, Chang S L, Feng F Y, Palapattu G S, Taichman R S, Chinnaiyan A M, Tomlins S A, Morgan T M. Multigene Profiling of CTCs in mCRPC Identifies a Clinically Relevant Prognostic Signature. Mol Cancer Res. 2018 April; 16(4):643-654. doi: 10.1158/1541-7786.MCR-17-0539. Epub 2018 Feb. 16. PMID:29453313
29. Spratt D E, Zhang J, Santiago-Jiménez M, Dess R T, Davis J W, Den R B, Dicker A P, Kane C J, Pollack A, Stoyanova R, Abdollah F, Ross A E, Cole A, Uchio E, Randall J M, Nguyen H, Zhao S G, Mehra R, Glass A G, Lam LLC, Chelliserry J, du Plessis M, Choeurng V, Aranes M, Kolisnik T, Margrave J, Alter J, Jordan J, Buerki C, Yousefi K, Haddad Z, Davicioni E, Trabulsi E J, Loeb S, Tewari A, Carroll P R, Weinmann S, Schaeffer E M, Klein E A, Karnes R J, Feng F Y, Nguyen P L. Development and Validation of a Novel Integrated Clinical-Genomic Risk Group Classification for Localized Prostate Cancer. J Clin Oncol. 2018 Feb. 20; 36(6):581-590. doi: 10.1200/JCO.2017.74.2940. Epub 2017 Nov. 29.PMID:29185869
30. Nguyen P L, Haddad Z, Ross A E, Martin N E, Deheshi S, Lam LLC, Chelliserry J, Tosoian J J, Lotan T L, Spratt D E, Stoyanova R S, Punnen S, Ong K, Buerki C, Aranes M, Kolisnik T, Margrave J, Yousefi K, Choeurng V, Davicioni E, Trock B J, Kane C J, Pollack A, Davis J W, Feng F Y, Klein E A. Ability of a Genomic Classifier to Predict Metastasis and Prostate Cancer-specific Mortality after Radiation or Surgery based on Needle Biopsy Specimens. Eur Urol. 2017 November; 72(5):845-852. doi: 10.1016/j.eururo.2017.05.009. Epub 2017 May 19. PMID:28528811
31. Klaassen Z, Howard L E, de Hoedt A, Amling C L, Aronson W J, Cooperberg M R, Kane C J, Terris M K, Freedland S J. Factors predicting skeletal-related events in patients with bone metastatic castration-resistant prostate cancer. Cancer. 2017 May 1; 123(9):1528-1535. doi: 10.1002/cncr.30505. Epub 2016 Dec. 27. PMID:28026865
32. Kohli M, Wang L, Xie F, Sicotte H, Yin P, Dehm S M, Hart S N, Vedell P T, Barman P, Qin R, Mahoney D W, Carlson R E, Eckel-Passow J E, Atwell T D, Eiken P W, McMenomy B P, Wieben E D, Jha G, Jimenez R E, Weinshilboum R, Wang L. Mutational Landscapes of Sequential Prostate Metastases and Matched Patient Derived Xenografts during Enzalutamide Therapy. PLoS One. 2015 Dec. 22; 10(12):e0145176. doi: 10.1371/journal.pone.0145176. eCollection 2015.PMID:26695660
33. Lee G T, Kang D I, Ha Y S, Jung Y S, Chung J, Min K, Kim T H, Moon K H, Chung J M, Lee D H, Kim W J, Kim I Y. Prostate cancer bone metastases acquire resistance to androgen deprivation via WNT5A-mediated BMP-6 induction. Br J Cancer. 2014 Mar. 18; 110(6):1634-44. doi: 10.1038/bjc.2014.23. Epub 2014 Feb. 11. PMID:24518599
34. Lee G T, Kwon S J, Kim J, Kwon Y S, Lee N, Hong J H, Jamieson C, Kim W J, Kim I Y. WNT5A induces castration-resistant prostate cancer via CCL2 and tumour-infiltrating macrophages. Br J Cancer. 2018 Mar. 6; 118(5):670-678. doi: 10.1038/bjc.2017.451. Epub 2018 Jan. 30. PMID:29381686
35. Miyamoto D T, Zheng Y, Wittner B S, Lee R J, Zhu H, Broderick K T, Desai R, Fox D B, Brannigan B W, Trautwein J, Arora K S, Desai N, Dahl D M, Sequist L V, Smith M R, Kapur R, Wu C L, Shioda T, Ramaswamy S, Ting D T, Toner M, Maheswaran S, Haber D A. RNA-Seq of single prostate CTCs implicates noncanonical Wnt signaling in antiandrogen resistance. Science. 2015 Sep. 18; 349(6254):1351-6. doi: 10.1126/science.aab0917. PMID: 26383955
36. F. Jin et al., “Regulation of prostate cancer cell migration toward bone marrow stromal cell-conditioned medium by Wnt5a signaling.,” Mol. Med. Rep., vol. 8, no. 5, pp. 1486-92, November 2013.
37. Tamura K, Furihata M, Satake H, Hashida H, Kawada C, Osakabe H, Fukuhara H, Kumagai N, Iiyama T, Shuin T, Inoue K.SHISA2 enhances the aggressive phenotype in prostate cancer through the regulation of WNT5A expression. Oncol Lett. 2017 December; 14(6):6650-6658. doi: 10.3892/ol.2017.7099. Epub 2017 Sep. 28. PMID: 29344118
38. Huang L, Pu Y, Hu W Y, Birch L, Luccio-Camelo D, Yamaguchi T, Prins G S. The role of Wnt5a in prostate gland development. Dev Biol. 2009 Apr. 15; 328(2):188-99. doi: 10.1016/j.ydbio.2009.01.003. Epub 2009 Jan. 14. PMID: 19389372
39. M. Okamoto et al., “Noncanonical Wnt5a enhances Wnt/0-catenin signaling during osteoblastogenesis.,” Sci. Rep., vol. 4, no. 1, p. 4493, March 2014.
40. Hosseini-Farahabadi S, Gignac S J, Danescu A, Fu K, Richman J M. Abnormal WNT5A Signaling Causes Mandibular Hypoplasia in Robinow Syndrome. J Dent Res. 2017 October; 96(11):1265-1272. doi: 10.1177/0022034517716916. Epub 2017 Jun. 29. PMID:28662348
41. Choi M Y, Widhopf G F 2nd, Wu C C, Cui B, Lao F, Sadarangani A, Cavagnaro J, Prussak C, Carson D A, Jamieson C, Kipps T J. Pre-clinical Specificity and Safety of UC-961, a First-In-Class Monoclonal Antibody Targeting ROR1. Clin Lymphoma Myeloma Leuk. 2015 June; 15 Suppl:S167-9. doi: 10.1016/j.clml.2015.02.010. Review. PMID: 26297272
42. Yu J, Chen L, Cui B, Wu C, Choi M Y, Chen Y, Zhang L, Rassenti L Z, Widhopf Ii G F, Kipps T J. Cirmtuzumab inhibits Wnt5a-induced Rac1 activation in chronic lymphocytic leukemia treated with ibrutinib. Leukemia. 2017 June; 31(6):1333-1339. doi: 10.1038/leu.2016.368. Epub 2016 Dec. 1. PMID: 27904138
43. Jamieson, C H, (P I), and Choi, M (Co-P I), Kipps, T J (Responsible Party), ClinicalTrials.gov Identifier: NCT02222688, A Phase I Clinical Trial to Determine the Safety and Tolerability of UC-961 (Cirmtuzumab), an Anti-ROR1 Monoclonal Antibody, for the Treatment of Patients With Relapsed or Refractory Chronic Lymphocytic Leukemia Who Are Ineligible for Chemotherapy. https://clinicaltrials.gov/ct2/show/NCT02222688
44. Bauer M, Bénard J, Gaasterland T, Willert K, Cappellen D. WNT5A encodes two isoforms with distinct functions in cancers. PLoS One. 2013 8 (11): e80526. doi:10.1371/journal.pone.0080526. PMID 24260410.
45. Raheem, O., A. A. Kulidjian, C. Wu, Y. B. Jeong, T. Yamaguchi, K. M. Smith, D. Goff, H. Leu, S. R. Morris, N. A. Cacalano, K. Masuda, C. H. Jamieson, C. J. Kane, and C. A. Jamieson, A novel patient-derived intra-femoral xenograft model of bone metastatic prostate cancer that recapitulates mixed osteolytic and osteoblastic lesions. J Transl Med, 2011. 9: p. 185.
46. Godebu, E., M. Muldong, A. Strasner, C. N. Wu, S. C. Park, J. R. Woo, W. Ma, M. A. Liss, T. Hirata, O. Raheem, N. A. Cacalano, A. A. Kulidjian, and C. A. Jamieson, PCSD1, a new patient-derived model of bone metastatic prostate cancer, is castrate-resistant in the bone-niche. J Transl Med, 2014. 12: p. 275.
47. Hirata, T., S. C. Park, M. T. Muldong, C. N. Wu, T. Yamaguchi, A. Strasner, O. Raheem, H. Kumon, R. L. Sah, N. A. Cacalano, C. H. M. Jamieson, C. J. Kane, K. Masuda, A. A. Kulidjian, and C. A. M. Jamieson, Specific bone region localization of osteolytic versus osteoblastic lesions in a patient-derived xenograft model of bone metastatic prostate cancer. Asian J Urol, 2016. 3(4): p. 229-239
48. CAM Jamieson, Kulidjian, A. A., Muldong, M, Wu, C., Lee, S., Mendoza, T., Park, S C, Park, J S, Raheem, O., Woo, J R, Godebu, E., Strasner, A., Kane, C J, Gaasterland, T. Genomic analysis of a longitudinal series of surgical prostate cancer bone metastases and xenografts from the same patient with progressively therapy-resistant cancer revealed selection of metastatic clone. (Research completed, Manuscript in Preparation).
49. Ritter K E, Wang Z, Vezina C M, Bjorling D E, Southard-Smith EM1.Serotonin Receptor 5-HT3A Affects Development of Bladder Innervation and Urinary Bladder Function. Front Neurosci. 2017 Dec. 12; 11:690. doi: 10.3389/fnins.2017.00690. eCollection 2017.
50. Hu W Y, Hu D P, Xie L, Li Y, Majumdar S, Nonn L, Hu H, Shioda T, Prins G S. Isolation and functional interrogation of adult human prostate epithelial stem cells at single cell resolution. Stem Cell Res. 2017 August; 23:1-12. doi: 10.1016/j.scr.2017.06.009. Epub 2017 Jun. 16. PMID: 28651114
51. Sarin S, Zuniga-Sanchez E, Kurmangaliyev Y Z, Cousins H, Patel M, Hernandez J, Zhang K X, Samuel M A, Morey M, Sanes J R, Zipursky S L. Role for Wnt Signaling in Retinal Neuropil Development: Analysis via RNA-Seq and In Vivo Somatic CRISPR Mutagenesis. Neuron. 2018 Apr. 4; 98(1):109-126.e8. doi: 10.1016/j.neuron.2018.03.004. Epub 2018 Mar. 22. PMID:29576390
52. Yu J, Chen L, Chen Y, Hasan M K, Ghia E M, Zhang L, Wu R, Rassenti L Z, Widhopf G F, Shen Z, Briggs S P, Kipps T J. Wnt5a induces ROR1 to associate with 14-3-3(for enhanced chemotaxis and proliferation of chronic lymphocytic leukemia cells. Leukemia. 2017 December; 31(12):2608-2614. doi: 10.1038/leu.2017.132. Epub 2017 May 3. PMID:28465528
53. Zhang S, Zhang H, Ghia E M, Huang J, Wu L, Zhang J, Lam S, Lei Y, He J, Cui B, Widhopf G F 2nd, Yu J, Schwab R, Messer K, Jiang W, Parker B A, Carson D A, Kipps T J. Inhibition of chemotherapy resistant breast cancer stem cells by a ROR1 specific antibody. Proc Natl Acad Sci USA. 2019 Jan. 22; 116(4):1370-1377. doi: 10.1073/pnas.1816262116. Epub 2019 Jan. 8.PMID:30622177
54. Yu J, Chen L, Cui B, Widhopf G F 2nd, Shen Z, Wu R, Zhang L, Zhang S, Briggs S P, Kipps T J. Wnt5a induces ROR1/ROR2 heterooligomerization to enhance leukemia chemotaxis and proliferation. J Clin Invest. 2016 February; 126(2):585-98. PMID:26690702
55. Hasan M K, Rassenti L, Widhopf G F 2nd, Yu J, Kipps T J. Wnt5a causes ROR1 to complex and activate cortactin to enhance migration of chronic lymphocytic leukemia cells. Leukemia. 2019 March; 33(3):653-661. doi: 10.1038/s41375-018-0306-7. Epub 2018 Dec. 19. PMID:30568170
56. Choi M Y, Widhopf G F 2nd, Ghia E M, Kidwell R L, Hasan M K, Yu J, Rassenti L Z, Chen L, Chen Y, Pittman E, Pu M, Messer K, Prussak C E, Castro J E, Jamieson C, Kipps T J. Phase I Trial: Cirmtuzumab Inhibits ROR1 Signaling and Stemness Signatures in Patients with Chronic Lymphocytic Leukemia. Cell Stem Cell. 2018 Jun. 1; 22(6):951-959.e3. doi: 10.1016/j.stem.2018.05.018.PMID:29859176
57. Zhang S, Chen L, Wang-Rodriguez J, Zhang L, Cui B, Frankel W, Wu R, Kipps T J. The onco-embryonic antigen ROR1 is expressed by a variety of human cancers. Am J Pathol. 2012 December; 181(6):1903-10. doi: 10.1016/j.ajpath.2012.08.024. Epub 2012 Oct. 4. PMID:23041612
58. Li C, Wang S, Xing Z, Lin A, Liang K, Song J, Hu Q, Yao J, Chen Z, Park P K, Hawke D H, Zhou J, Zhou Y, Zhang S, Liang H, Hung M C, Gallick G E, Han L, Lin C, Yang L. A ROR1-HER3-lncRNA signalling axis modulates the Hippo-YAP pathway to regulate bone metastasis. Nat Cell Biol. 2017 February; 19(2):106-119. doi: 10.1038/ncb3464. Epub 2017 Jan. 23.PMID:28114269
59. Woo J R, Liss M A, Muldong M T, Palazzi K, Strasner A, Ammirante M, Varki N, Shabaik A, Howell S, Kane C J, Karin M, Jamieson C A. Tumor infiltrating B-cells are increased in prostate cancer tissue. J Transl Med. 2014 Jan. 30; 12:30. doi: 10.1186/1479-5876-12-30. PMID:24475900

1-73. (canceled)

74. A chimeric antigen receptor comprising:
(i) an antigen binding region comprising a light chain variable domain and a heavy chain variable domain; wherein:
(a) the light chain variable domain comprises a CDR L1 as set forth in SEQ ID NO:43, a CDR L2 as set forth in SEQ ID NO:44 and a CDR L3 as set forth in SEQ ID NO:45; and the heavy chain variable domain comprises a CDR H1 as set forth in SEQ ID NO:46, a CDR H2 as set forth in SEQ ID NO:47, and a CDR H3 as set forth in SEQ ID NO:48; or
(b) the light chain variable domain comprises a CDR L1 as set forth in SEQ ID NO:49, a CDR L2 as set forth in SEQ ID NO:50 and a CDR L3 as set forth in SEQ ID NO:51; and the heavy chain variable domain comprising a CDR H1 as set forth in SEQ ID NO:52, a CDR H2 as set forth in SEQ ID NO:53, and a CDR H3 as set forth in SEQ ID NO:54;
(ii) a spacer domain;
(iii) a transmembrane domain; and
(iv) an intracellular domain.

75. The chimeric antigen receptor of claim 74, wherein the light chain variable domain is covalently linked to the heavy chain variable domain through a polypeptide linker.

76. The chimeric antigen receptor of claim 74, wherein the spacer domain comprises an immunoglobulin hinge domain, an immunoglobulin constant heavy chain 3 domain, an immunoglobulin constant heavy chain 2 domain, or a combination thereof.

77. The chimeric antigen receptor of claim 74, wherein the spacer domain comprises an amino acid sequence of SEQ ID NO:29, SEQ ID NO:41 or SEQ ID NO:42.

78. The chimeric antigen receptor of claim 74, wherein the light chain variable domain comprises an amino acid sequence at least about 95% identical to SEQ ID NO:21.

79. The chimeric antigen receptor of claim 74, wherein the heavy chain variable domain comprises an amino acid sequence at least about 95% identical to SEQ ID NO: 27.

80. The chimeric antigen receptor of claim 74, wherein the transmembrane domain comprises a CD8α transmembrane domain, a CD28 transmembrane domain, a CD4 transmembrane domain, a CD3ζ transmembrane domain, or a combination of two or more thereof.

81. The chimeric antigen receptor of claim 74, wherein the intracellular domain comprises an intracellular co-stimulatory signaling domain, an intracellular T-cell signaling domain, or a combination thereof.

82. The chimeric antigen receptor of claim 81, wherein the intracellular co-stimulatory signaling domain comprises a 4-1BB intracellular co-stimulatory signaling domain, a CD28 intracellular co-stimulatory signaling domain, a ICOS intracellular co-stimulatory signaling domain, an OX-40 intracellular co-stimulatory signaling domain, or a combination of two or more thereof.

83. The chimeric antigen receptor of claim 82, wherein the intracellular costimulatory signaling domain comprises a CD28 intracellular co-stimulatory signaling domain and a 4-1BB intracellular co-stimulatory signaling domain.

84. The chimeric antigen receptor of claim 83, wherein the intracellular costimulatory signaling domain further comprises a CD3ζ intracellular T-cell signaling domain.

85. A cell that expresses the chimeric antigen receptor of claim 74.

86. The cell of claim 85, wherein the cell is a T lymphocyte.

87. The cell of claim 85, wherein the cell is a CD4+T lymphocyte or a CD8+T lymphocyte.

88. The cell of claim 85, wherein the cell is a natural killer cell.

89. A pharmaceutical composition comprising a cell that expresses the chimeric antigen receptor of claim 74 and a pharmaceutically acceptable excipient.

90. An isolated nucleic acid encoding the chimeric antigen receptor of claim 74.

91. A cell comprising a nucleic acid that encodes the chimeric antigen receptor of claim 74.

92. A method of treating a neuroendocrine cancer in a subject in need thereof, treating metastasis in a subject with a neuroendocrine cancer in need thereof, or preventing metastasis in a subject with a neuroendocrine cancer in need thereof, the method comprising administering to the subject an effective amount of a cell that expresses the chimeric antigen receptor of claim 74.

93. The method of claim 92, wherein the neuroendocrine cancer is a carcinoid tumor, an islet cell tumor, a medullary thyroid cancer, a pheochromocytoma, a neuroendocrine carcinoma of the skin, small cell lung cancer, large cell neuroendocrine carcinoma, neuroendocrine prostate cancer, or metastatic castration-resistant prostate cancer.
